SEQUENCE LISTING (77 sequences)

SEQ ID NO: 1 - DNA, 21 bp, Artificial Sequence
  Description of Artificial Sequence Synthetic humanized antibody oligonucleotide
tatgcaaggcttacaaccaca

SEQ ID NO: 2 - DNA, 36 bp, Artificial Sequence
  Description of Artificial Sequence Synthetic primer
ggccacgcgtcgactagtacgggnngggnngggnng

SEQ ID NO: 3 - DNA, 26 bp, Artificial Sequence
  Description of Artificial Sequence Synthetic primer
cacaattttcttgtccaccttggtgc

SEQ ID NO: 4 - DNA, 18 bp, Artificial Sequence
  Description of Artificial Sequence Synthetic primer
ccttgaccaggcatccca

SEQ ID NO: 5 - DNA, 27 bp, Artificial Sequence
  Description of Artificial Sequence Synthetic primer
ctcattcctgttgaagctcttgacaat

SEQ ID NO: 6 - DNA, 30 bp, Artificial Sequence
  Description of Artificial Sequence Synthetic primer
ctcattcctgttgaagctcttgacaatggg

SEQ ID NO: 7 - DNA, 21 bp, Artificial Sequence
  Description of Artificial Sequence Synthetic primer
agttgttcaagaagcacacga

SEQ ID NO: 8 - protein, 10 aa, Mus musculus
GYTFTDYYIH

SEQ ID NO: 9 - protein, 17 aa, Mus musculus
RVNPNNGGTRYNQKFED

SEQ ID NO: 10 - protein, 12 aa, Mus musculus
SPLYYYDGRSGY

SEQ ID NO: 11 - protein, 10 aa, Mus musculus
RASSDVRYMY

SEQ ID NO: 12 - protein, 7 aa, Mus musculus
YTSNLAS

SEQ ID NO: 13 - protein, 9 aa, Mus musculus
QQFTTSPWT

SEQ ID NO: 14 - protein, 5 aa, Mus musculus
DYYIH

SEQ ID NO: 15 - protein, 7 aa, Mus musculus
GYTFTDY

SEQ ID NO: 16 - protein, 11 aa, Mus musculus
GYSITSSYSWN

SEQ ID NO: 17 - protein, 16 aa, Mus musculus
NIYYSGSTNYNPSLKS

SEQ ID NO: 18 - protein, 3 aa, Artificial Sequence
  Description of Artificial Sequence Synthetic peptide
PRV

SEQ ID NO: 19 - protein, 11 aa, Mus musculus
RASQEISGYLG

SEQ ID NO: 20 - protein, 7 aa, Mus musculus
AASTLDS

SEQ ID NO: 21 - protein, 9 aa, Mus musculus
LQYASFPRT

SEQ ID NO: 22 - protein, 6 aa, Mus musculus
SSYSWN

SEQ ID NO: 23 - protein, 8 aa, Mus musculus
GYSITSSY

SEQ ID NO: 24 - protein, 10 aa, Mus musculus
GYIFTNYWMH

SEQ ID NO: 25 - protein, 17 aa, Mus musculus
RIHPSDSDTDYNQKFKG

SEQ ID NO: 26 - protein, 8 aa, Mus musculus
TLKWDVGY

SEQ ID NO: 27 - protein, 10 aa, Mus musculus
SASSSINSTY

SEQ ID NO: 28 - protein, 7 aa, Mus musculus
RTSTLAS

SEQ ID NO: 29 - protein, 9 aa, Mus musculus
QQWSSYPLT

SEQ ID NO: 30 - protein, 10 aa, Mus musculus
SASSSINSMY

SEQ ID NO: 31 - protein, 10 aa, Mus musculus
GYTFTSYWMH

SEQ ID NO: 32 - protein, 17 aa, Mus musculus
RILPSNSDTIYNQKFKD

SEQ ID NO: 33 - protein, 8 aa, Mus musculus
TLNWDVGY

SEQ ID NO: 34 - protein, 10 aa, Mus musculus
SASSSVSSMY

SEQ ID NO: 35 - protein, 7 aa, Mus musculus
RTSKLAS

SEQ ID NO: 36 - protein, 9 aa, Mus musculus
QQWSSNPLT

SEQ ID NO: 37 - protein, 10 aa, Artificial Sequence
  Description of Artificial Sequence Synthetic consensus CDR peptide
GYXFTXYXXH

SEQ ID NO: 38 - protein, 17 aa, Artificial Sequence
  Description of Artificial Sequence Synthetic consensus CDR peptide
RXXPXXXXTXYNQKFXX

SEQ ID NO: 39 - protein, 8 aa, Artificial Sequence
  Description of Artificial Sequence Synthetic consensus CDR peptide
TLXWDVGY

SEQ ID NO: 40 - protein, 10 aa, Artificial Sequence
  Description of Artificial Sequence Synthetic consensus CDR peptide
XASSXXXXXY

SEQ ID NO: 41 - protein, 7 aa, Artificial Sequence
  Description of Artificial Sequence Synthetic consensus CDR peptide
XTSXLAS

SEQ ID NO: 42 - protein, 9 aa, Artificial Sequence
  Description of Artificial Sequence Synthetic consensus CDR peptide
QQXXXXPXT

SEQ ID NO: 43 - protein, 121 aa, Mus musculus
EVQLQQSGPEMVKPGASVKISCKTSGYTFTDYYIHWVKQSHGKSLEWIGRVNPNNGGTRYNQKFEDKATLTVDKSPSTAYMELNSLTSEDSAVYYCAISPLYYYDGRSGYWGQGTTLTVSS

SEQ ID NO: 44 - protein, 106 aa, Mus musculus
ENVLTQSPAILSATLGEKVTMSCRASSDVRYMYWHQQKSGASPKLWIYYTSNLASGVPARFSGSGSGTSYSLTISSVEAEDAATYYCQQFTTSPWTFGGGTKLEIK

SEQ ID NO: 45 - protein, 112 aa, Mus musculus
DVQLQESGPGLVKPSQSLSVTCTVTGYSITSSYSWNWIRQFPGNKLEWMGNIYYSGSTNYNPSLKSRISITRDTSKNQFFLQLNSVTTEDTATYYCANPRVWGAGTTVTVSS

SEQ ID NO: 46 - protein, 107 aa, Mus musculus
DIQMTQSPSSLSASLGERVSLTCRASQEISGYLGWLQQKPDGTIKRLIYAASTLDSGVPKRFSGSRSGSDYSLTISSLESEDFADYYCLQYASFPRTFGGGTKLEIQ

SEQ ID NO: 47 - protein, 117 aa, Mus musculus
QVQLQQPGAELVKPGASLRVSCRASGYIFTNYWMHWVKQRPGQGLEWIGRIHPSDSDTDYNQKFKGKATLTVDKSSSTVYMQLSSLTSADSAVYYCATTLKWDVGYWGQGTTLTVSS

SEQ ID NO: 48 - protein, 106 aa, Mus musculus
QIVLTQSPTIMSASPGEKVTMTCSASSSINSTYWYQQKPGSSPKPWIYRTSTLASGVPVRFSGSGSGTSYSLTISSMEAEDAATYYCQQWSSYPLTFGAGTKLEMK

SEQ ID NO: 49 - protein, 106 aa, Mus musculus
QIVLTQSPTIMSASPGEKVTMTCSASSSINSMYWYQQKPGSSPKPWIYRTSTLASGVPVRFSGSGSGTSYSLTISSMEAEDAATYYCQQWSSYPLTFGAGTKLEMK

SEQ ID NO: 50 - protein, 117 aa, Mus musculus
QVQLQQPGAELVKPGASVKVSCKTSGYTFTSYWMHWVKQRPGQGLEWIGRILPSNSDTIYNQKFKDKATLTVDKSSSTVYMQLTSLTSEDSAVYYCAITLNWDVGYWGQGTTLTVSS

SEQ ID NO: 51 - protein, 106 aa, Mus musculus
QIVLTQSPAIMSASPGEKVTMTCSASSSVSSMYWYQQKPGSSPRPWICRTSKLASGVPVRFSGSGSGTSYSLTISSMEAEDAATYYCQQWSSNPLTFGAGTKLEMK

SEQ ID NO: 52 - protein, 140 aa, Artificial Sequence
  Description of Artificial Sequence Synthetic humanized antibody polypeptide
KLAATMKHLWFFLLLVAAPRWVLSQVQLQESGPGLVKPSETLSLTCTVSGYSITSSYSWNWIRQPPGKGLEWIGNIYYSGSTNYNPSLKSRVTISVDTSKNQFSLKLSSVTAADTAVYYCARPRVWGQGTTVTVSSASTK

SEQ ID NO: 53 - DNA, 425 bp, Artificial Sequence
  Description of Artificial Sequence Synthetic humanized antibody polynucleotide
aagcttgccgccaccatgaaacatctgtggttcttccttctcctggtggcagctcccagatgggtcctgtcccaagtgcagttgcaggaatcaggcccaggcctggtgaaaccaagcgagacactgagcttgacctgcactgtgtccggttactcaatcacctcctcttacagctggaactggatcaggcagccacctggaaagggccttgagtggatcgggaatatctattactctggctccactaactataatccttccctgaaatccagggtgaccatttctgttgatacaagtaaaaaccagttctctcttaaactttctagtgtgactgcagcagatacagcagtctattattgtgcccgaccccgggtttggggccagggaaccactgtaaccgtttcttctgccagcaccaagggccc

SEQ ID NO: 54 - protein, 112 aa, Artificial Sequence
  Description of Artificial Sequence Synthetic humanized antibody polypeptide
QVQLQESGPGLVKPSETLSLTCTVSGYSITSSYSWNWIRQPPGKGLEWIGNIYYSGSTNYNPSLKSRVTISVDTSKNQFSLKLSSVTAADTAVYYCARPRVWGQGTTVTVSS

SEQ ID NO: 55 - DNA, 408 bp, Artificial Sequence
  Description of Artificial Sequence Synthetic humanized antibody polynucleotide
aagcttgccgccaccatggacatgagggtccccgctcagctcctggggctcctgctactctggctccgaggtgccagatgtgacatccagatgacacagtcaccatcttcccttagcgcctctgttggcgaccgcgtcaccattacttgtagagctagccaggagatttctggctatctcggctggtatcaacaaaaacccggtaaagctccaaagctgctcatctatgctgctagcactcttgactctggtgttccatctcgcttctcaggtagtgggtccgggactgatttcaccctcactatttctagcctgcagcctgaagacttcgccacttactattgcctgcagtacgcatctttcccacggacatttggacagggcaccaaacttgagataaagcgtacg

SEQ ID NO: 56 - protein, 136 aa, Artificial Sequence
  Description of Artificial Sequence Synthetic humanized antibody polypeptide
KLAATMDMRVPAQLLGLLLLWLRGARCDIQMTQSPSSLSASVGDRVTITCRASQEISGYLGWYQQKPGKAPKLLIYAASTLDSGVPSRFSGSGSGTDFTLTISSLQPEDFATYYCLQYASFPRTFGQGTKLEIKRT

SEQ ID NO: 57 - protein, 107 aa, Artificial Sequence
  Description of Artificial Sequence Synthetic humanized antibody polypeptide
DIQMTQSPSSLSASVGDRVTITCRASQEISGYLGWYQQKPGKAPKLLIYAASTLDSGVPSRFSGSGSGTDFTLTISSLQPEDFATYYCLQYASFPRTFGQGTKLEIK

SEQ ID NO: 58 - protein, 112 aa, Artificial Sequence
  Description of Artificial Sequence Synthetic humanized antibody polypeptide
QVQLQESGPGLVKPSETLSVTCTVSGYSITSSYSWNWIRQPPGKGLEWIGNIYYSGSTNYNPSLKSRVTISVDTSKNQFSLKLSSVTAADTAVYYCARPRVWGQGTTVTVSS

SEQ ID NO: 59 - protein, 112 aa, Artificial Sequence
  Description of Artificial Sequence Synthetic humanized antibody polypeptide
QVQLQESGPGLVKPSETLSLTCTVSGYSITSSYSWNWIRQFPGKGLEWIGNIYYSGSTNYNPSLKSRVTISVDTSKNQFSLKLSSVTAADTAVYYCARPRVWGQGTTVTVSS

SEQ ID NO: 60 - protein, 112 aa, Artificial Sequence
  Description of Artificial Sequence Synthetic humanized antibody polypeptide
QVQLQESGPGLVKPSETLSLTCTVSGYSITSSYSWNWIRQPPGKGLEWIGNIYYSGSTNYNPSLKSRISISVDTSKNQFSLKLSSVTAADTAVYYCARPRVWGQGTTVTVSS

SEQ ID NO: 61 - protein, 112 aa, Artificial Sequence
  Description of Artificial Sequence Synthetic humanized antibody polypeptide
QVQLQESGPGLVKPSETLSLTCTVSGYSITSSYSWNWIRQPPGKGLEWIGNIYYSGSTNYNPSLKSRVTISRDTSKNQFSLKLSSVTAADTAVYYCARPRVWGQGTTVTVSS

SEQ ID NO: 62 - protein, 112 aa, Artificial Sequence
  Description of Artificial Sequence Synthetic humanized antibody polypeptide
QVQLQESGPGLVKPSETLSLTCTVSGYSITSSYSWNWIRQPPGKGLEWIGNIYYSGSTNYNPSLKSRVTISVDTSKNQFSLKLSSVTAADTAVYYCANPRVWGQGTTVTVSS

SEQ ID NO: 63 - protein, 112 aa, Artificial Sequence
  Description of Artificial Sequence Synthetic humanized antibody polypeptide
QVQLQESGPGLVKPSETLSVTCTVSGYSITSSYSWNWIRQFPGKGLEWIGNIYYSGSTNYNPSLKSRISISRDTSKNQFSLKLSSVTAADTAVYYCANPRVWGQGTTVTVSS

SEQ ID NO: 64 - protein, 112 aa, Artificial Sequence
  Description of Artificial Sequence Synthetic humanized antibody polypeptide
QVQLQESGPGLVKPSETLSVTCTVSGYSITSSYSWNWIRQFPGKGLEWIGNIYYSGSTNYNPSLKSRVTISRDTSKNQFSLKLSSVTAADTAVYYCANPRVWGQGTTVTVSS

SEQ ID NO: 65 - protein, 112 aa, Artificial Sequence
  Description of Artificial Sequence Synthetic humanized antibody polypeptide
QVQLQESGPGLVKPSETLSLTCTVSGYSITSGYSWNWIRQPPGKGLEWIGNIYYSGSTNYNPSLKSRVTISVDTSKNQFSLKLSSVTAADTAVYYCANPRVWGQGTTVTVSS

SEQ ID NO: 66 - protein, 107 aa, Artificial Sequence
  Description of Artificial Sequence Synthetic humanized antibody polypeptide
DIQMTQSPSSLSASVGDRVTITCRASQEISGYLGWLQQKPGKAPKLLIYAASTLDSGVPSRFSGSGSGTDFTLTISSLQPEDFATYYCLQYASFPRTFGQGTKLEIK

SEQ ID NO: 67 - protein, 107 aa, Artificial Sequence
  Description of Artificial Sequence Synthetic humanized antibody polypeptide
DIQMTQSPSSLSASVGDRVTITCRASQEISGYLGWYQQKPGKAIKLLIYAASTLDSGVPSRFSGSGSGTDFTLTISSLQPEDFATYYCLQYASFPRTFGQGTKLEIK

SEQ ID NO: 68 - protein, 107 aa, Artificial Sequence
  Description of Artificial Sequence Synthetic humanized antibody polypeptide
DIQMTQSPSSLSASVGDRVTITCRASQEISGYLGWYQQKPGKAPKRLIYAASTLDSGVPSRFSGSGSGTDFTLTISSLQPEDFATYYCLQYASFPRTFGQGTKLEIK

SEQ ID NO: 69 - protein, 107 aa, Artificial Sequence
  Description of Artificial Sequence Synthetic humanized antibody polypeptide
DIQMTQSPSSLSASVGDRVTITCRASQEISGYLGWYQQKPGKAPKLLIYAASTLDSGVPSRFSGSRSGTDFTLTISSLQPEDFATYYCLQYASFPRTFGQGTKLEIK

SEQ ID NO: 70 - protein, 107 aa, Artificial Sequence
  Description of Artificial Sequence Synthetic humanized antibody polypeptide
DIQMTQSPSSLSASVGDRVTITCRASQEISGYLGWLQQKPGKAIKRLIYAASTLDSGVPSRFSGSRSGTDFTLTISSLQPEDFATYYCLQYASFPRTFGQGTKLEIK

SEQ ID NO: 71 - protein, 107 aa, Artificial Sequence
  Description of Artificial Sequence Synthetic humanized antibody polypeptide
DIQMTQSPSSLSASVGDRVTITCRASQEISGYLGWLQQKPGKAIKRLIYAASTLDSGVPSRFSGSRSGTDYTLTISSLQPEDFATYYCLQYASFPRTFGQGTKLEIK

SEQ ID NO: 72 - protein, 107 aa, Artificial Sequence
  Description of Artificial Sequence Synthetic humanized antibody polypeptide
DIQMTQSPSSLSASVGDRVTITCRASQEISGYLGWLQQKPGKAPKRLIYAASTLDSGVPSRFSGSRSGTDFTLTISSLQPEDFATYYCLQYASFPRTFGQGTKLEIK

SEQ ID NO: 73 - protein, 107 aa, Artificial Sequence
  Description of Artificial Sequence Synthetic humanized antibody polypeptide
DIQMTQSPSSLSASVGDRVTITCRASQSISGYLGWLQQKPGKAIKRLIYAASTLDSGVPSRFSGSRSGTDFTLTISSLQPEDFATYYCLQYARFPRTFGQGTKLEIK

SEQ ID NO: 74 - protein, 11 aa, Artificial Sequence
  Description of Artificial Sequence Synthetic humanized antibody peptide
GYSITSGYSWN

SEQ ID NO: 75 - protein, 12 aa, Artificial Sequence
  Description of Artificial Sequence Synthetic humanized antibody peptide
RASQSISGYLGW

SEQ ID NO: 76 - protein, 9 aa, Artificial Sequence
  Description of Artificial Sequence Synthetic humanized antibody peptide
LQYARFPRT

SEQ ID NO: 77 - DNA, 25 bp, Artificial Sequence
  Description of Artificial Sequence Synthetic oligonucleotide
ttttttttttttttttttttttttt

(19) United States
(12) Patent Application Publication
(10) Pub. No.: US 2015/0152172 A1
(43) Pub. Date: Jun. 4, 2015

(54) COMPOSITIONS AND METHODS FOR BINDING CYSTEINYL LEUKOTRIENES (CYSLTS) FOR TREATMENT OF DISEASE

(71) Applicant: LPATH, INC., SAN DIEGO, CA (US)

(72) Inventors: Jonathan Michael WOJCIAK, Encinitas, CA (US); Cindy Takeuchi DICKERSON, Encinitas, CA (US); Barbara VISENTIN, Del Mar, CA (US); Roger A. SABBADINI, Lakeside, CA (US)

(21) Appl. No.: 14/523,784

(22) Filed: Oct. 24, 2014

Related U.S. Application Data
(60) Provisional application No. 61/895,896, filed on Oct. 25, 2013.
(60) Provisional application No. 61/909,845, filed on Nov. 27, 2013.

(51) Int. Cl. C07K 16/18; A61K 47/48; A61K 39/00

(52) U.S. Cl. CPC C07K 16/18; A61K 39/0005; A61K 47/48238; C07K 2317/565; C07K 2317/24; A61K 2039/505; C07K 2317/31; A61K 2039/6031

(57) ABSTRACT

Methods are provided for using antibodies that bind one or more cysteinyl leukotrienes (cysLTs) for treatment of diseases, including inflammatory diseases and asthma, associated with aberrant levels of one or more cysLTs. Anti-cysLT antibodies and antigen-binding antibody fragments, and compositions containing such antibodies and antibody fragments, are also provided.

### RELATED APPLICATIONS

This application claims the benefit of and priority to U.S. provisional patent application Ser. Nos. 61/895,896 filed 25 Oct. 2013 and 61/909,845 filed 27 Nov. 2013; attorney docket numbers LPT-3500-PV and LPT-3500-PV2, each of which is hereby incorporated by reference in its entirety for any and all purposes.

### TECHNICAL FIELD

The present invention relates to methods of treating diseases, including diseases characterized by airway inflammation, using antibodies that bind cysteinyl-leukotrienes (cysLTs). This invention also relates to antibodies, particularly monoclonal antibodies, which bind one or more cysLTs.

### SEQUENCE LISTING

The instant application contains a Sequence Listing submitted via the Electronic Filing System on 24 Oct. 2014 and, is hereby incorporated by reference in its entirety. Said ASCII copy, created on 24 Oct. 2014 is named LPT3500UT.txt, and is 40,640 bytes in size.

### BACKGROUND OF THE INVENTION

1. Introduction

The following description includes information that may be useful in understanding the present invention. It is not an admission that any of the information provided herein, or any publication specifically or implicitly referenced herein, is prior art, or even particularly relevant, to the presently claimed invention.

2. Background

### Bioactive Signaling Lipids

Lipids and their derivatives are now recognized as important targets for medical research, not as just simple structural elements in cell membranes or as a source of energy for β-oxidation, glycolysis or other metabolic processes. In particular, certain bioactive lipids function as signaling mediators important in animal and human disease. Although most of the lipids of the plasma membrane play an exclusively structural role, a small proportion of them are involved in relaying extracellular stimuli into cells. These lipids are referred to as “bioactive lipids” or, alternatively, “bioactive signaling lipids.” “Lipid signaling” refers to any of a number of cellular signal transduction pathways that use cell membrane lipids as second messengers, as well as referring to direct interaction of a lipid signaling molecule with its own specific receptor. Lipid signaling pathways are activated by a variety of extracellular stimuli, ranging from growth factors to inflammatory cytokines, and regulate cell fate decisions such as apoptosis, differentiation and proliferation. Research into bioactive lipid signaling is an area of intense scientific investigation as more and more bioactive lipids are identified and their actions characterized.

Cysteinyl Leukotrienes (cysLTs)

Leukotrienes are a family of eicosanoid lipid mediators of inflammation produced in leukocytes by the oxidation of arachidonic acid by the enzyme arachidonate 5-lipoxygenase. These compounds have four double bonds, three of which are conjugated (hence the name “leukotriene”). Leukotrienes include leukotriene A4 (LTA4), leukotriene B4 (LTB4), leukotriene C4 (LTC4), leukotriene D4 (LTD4) and leukotriene E4 (LTE4) as well as leukotriene F4 (LTF4), which to date has only been produced synthetically (LTF4 Product Information Sheet, Item no. 20520, Cayman Chemical, Ann Arbor Mich.). Leukotriene G4 (LTG4) has also only been observed in in vitro conditions resulting from the transamination of LTE4, and has not been detected in intact animal tissues or fluids (LTG4 Product Information Sheet, Item no. 20610, Cayman Chemical, Ann Arbor Mich.).

Cysteinyl leukotrienes (cysLTs) are so named due to the presence of a cysteine residue in their structure. LTC4, LTD4, LTE4 and LTF4 are cysteinyl leukotrienes. Because LTF4 does not appear to be naturally occurring, typically “cysLTs”, in the context of disease and therapeutics, refers to LTC4, LTD4 and LTE4. In the context of this invention, “cysLT” or “cysLTs” refers to one or more cysteinyl leukotrienes that occur in nature and are correlated or associated with a disease or other unhealthful condition. Particularly preferred cysLT targets include LTC4, LTD4 and LTE4, the structures of which are shown below.

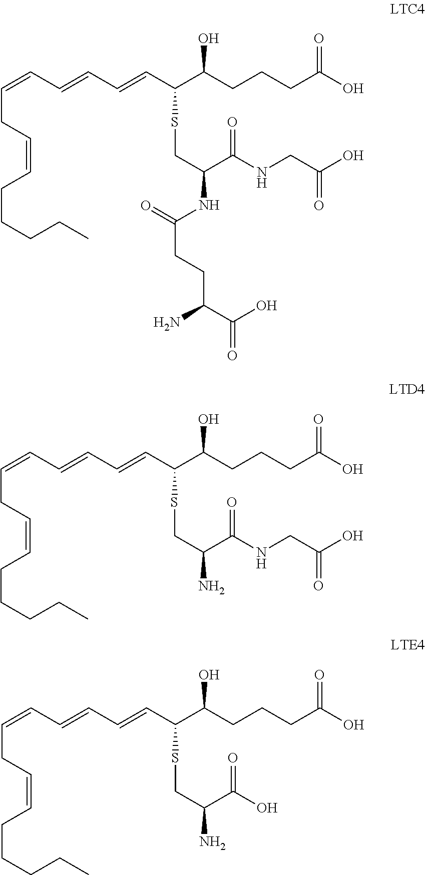 [SMILES: CCCCC/C=C\C\C=C/C=C/C=C/[C@@H](SC[C@H](NC(=O)CC[C@H](N)C(=O)O)C(=O)NCC(=O)O)[C@@H](O)CCCC(=O)O ; CCCCC/C=C\C\C=C/C=C/C=C/[C@@H](SC[C@H](N)C(=O)NCC(=O)O)[C@@H](O)CCCC(=O)O ; CCCCC/C=C\C\C=C/C=C/C=C/[C@@H](SC[C@H](N)C(=O)O)[C@@H](O)CCCC(=O)O]

As shown, the polar head groups of these cysLTs differ but the nonpolar hydrocarbon tails (shown on the left side of each diagram) are the same in all three compounds.

Synthesis of cysLTs

CysLTs are rapidly generated, e.g., at sites of inflammation, following a series of reactions resulting in the release of arachidonic acid. 5-Lipoxygenase (5-LO) uses 5-LO Activating Protein (FLAP) to convert arachidonic acid into 5-hydroperoxyeicosatetraenoic acid (5-HPETE), which spontaneously reduces to 5-hydroxyeicosatetraenoic acid (5-HETE). The enzyme 5-LO converts 5-HETE to convert it into leukotriene A4 (5S,6S-epoxy-7E,9E,11Z,14Z-eicosatetraenoic acid, LTA4), which is unstable. LTA4 is converted to the dihydroxy acid leukotriene B4 (5S,12R-dihydroxy-6Z,8E,10E,14Z-eicosatetraenoic acid, LTB4) by LTA4 hydrolase. LTB4 is a chemoattractant for neutrophils.

In cells expressing LTC4 synthase, such as eosinophils, basophils, mast cells and alveolar macrophages, LTA4 is conjugated with the tripeptide glutathione to form the first of the cysLTs, LTC4 (5S-hydroxy-6R—(S-glutathionyl)-7E,9E,11Z,14Z-eicosatetraenoic acid). Outside the cell, LTC4 can be converted by ubiquitous enzymes to form successively LTD4 (5S-hydroxy-6R—(S-cysteinylglycinyl)-7E,9E,11Z,14Z-eicosatetraenoic acid), by cleavage of the glutamic acid moiety. LTD4 can be converted to LTE4 (5S-hydroxy-6R—(S-cysteinyl)-7E,9E,11Z,14Z-eicosatetraenoic acid) by cleavage of the glycine moiety. LTC4, LTD4 and LTE4 have biological activity, though LTE4 is believed to be the most stable and abundant of the three.

CysLT Receptors

The cysLTs are potent biological mediators in the pathophysiology of inflammatory diseases and trigger contractile and inflammatory processes through the specific interaction with cell surface receptors, belonging to the superfamily of G-protein-coupled receptors. At present two cysLT receptors have been identified in both humans and mice, and are called CysLT1 and CysLT2. These two receptors are structurally divergent, having less than 40% amino acid homology in humans. Kanaoka, Y. and J. A. Boyce, (2004) J Immunol 173:1503-1510. CysLT1 receptor is believed to be most strongly expressed in spleen and peripheral blood leukocytes, and less strongly in lung, small intestine, colon, pancreas and placenta, and it has been implicated in airway inflammation, including asthma. Like CysLT1 receptors, CysLT2 receptors are expressed in spleen and peripheral blood leukocytes, but only CysLT2 receptors appear to be expressed in the heart, brain and adrenal glands. For review see Singh et al., (2010) Pharmacol 85:336-349.

The specificity of the two CysLT receptors seems to be different. The human CysLT1R is a high-affinity receptor for LTD4 whereas the human CysLT2R has equal affinity for LTC4 and LTD4; neither receptor has significant affinity for LTE4. The existence of an additional cys-LT receptor with a preference for LTE4 has long been suspected but one has not been definitively identified. It has been suggested that that the adenosine diphosphate (ADP)-reactive purinergic (P2Y12) receptor is required for the functions of LTE4. Paruchuri et al. (2009) J Exp Med 206: 2543-2555. The existence of a separate LTE4 receptor has also been reported by Maekawa et al (2008) Proc Natl Acad Sci 105:16695-16700.

CysLT1 receptor antagonists [e.g., montelukast (SINGULAIR™), zafirlukast, and pranlukast] have been developed and are widely prescribed for the prevention and chronic treatment of asthma, exercise-induced bronchioconstriction and allergic rhinitis. Because of its mechanism of action (i.e., blocking the action of LTD4, as well as LTC4 and LTE4, on CysLT1R), montelukast is generally not given as an acute treatment for asthma, but rather is often given as complementary therapy to inhaled corticosteroids. However, use of high doses of montelukast in acute asthma has been proposed. Wu et al. (2003) Clin & Exp Allergy 33:359-366.

CysLTs in Disease States

CysLTs have been shown to play a role in pathophysiological conditions, particularly inflammatory diseases and conditions including respiratory diseases and disorders such as asthma, allergic rhinitis and other allergies, and have been implicated in conditions including airway hyperresponsiveness, cardiovascular diseases, cerebrovascular disease, cancer, gastrointestinal conditions and skin conditions including atopic dermatitis and urticaria. CysLTs are powerful vasoconstrictors. Capra et al. (2007) Med Res Rev, 27:469-527, Riccioni, et al. (2008) J. Leukocyte Biol 84:1374-1378. Riccioni, G and M Bäck. Scientific World Journal, published online 2012 May 1. doi: 10.1100/2012/490968. Singh (2010) Pharmacol 85:336-349.

CysLT levels have been shown to be elevated in disease. For example, cysLT over-production is thought to be a key factor in the induction of eosinophilic activation. In AERD patients, elevation of CysLT levels in the urine, sputum, peripheral blood, and exhaled breath are observed after aspirin challenge. Leukotriene E4 has been shown to be more potent than other CysLTs, and contributes to the increase of histamine-induced airway responsiveness, eosinophic recruitment and resultant increases in vascular permeability (Palikhe, et al. (2009), Yonsei Med J 50:744-750). CysLTs are actively involved in the inflammation seen both in asthma and rhinitis. Inhalation of LTE4 has proven to be a very potent bronchoconstrictor and induces recruitment of inflammatory cells, especially eosinophils, into the tissue. Sputum from asthmatics had higher levels of Cys-LT compared with rhinitis patients or healthy controls. Tuvfesson, et al. (2007), Clin & Exp Allergy 37:1067-1073. Excretion of cysLTs has been reported after episodes of unstable angina and acute myocardial infarction, in coronary artery disease and after coronary artery bypass surgery, as well as in patients with atopic dermatitis, rheumatoid arthritis, Crohn's disease and malignant astrocytoma. Capra, ibid. “Slow-reacting substance of anaphylaxis” (SRS-A) is a mixture of LTC4, LTD4 and LTE4. Samuelsson, B. (1983), Science 220:568-575.

In the central nervous system, cysLTs are produced in response to a variety of acute brain injuries, such as stroke and traumatic brain injury (TBI). CysLT receptor antagonists have been shown to decrease infarct size after experimental cerebral artery occlusion, a model of stroke. LTC4 and LTD4 levels rise in a rat model of TBI, peaking about an hour after injury. Farias et al. (2009), J Neurotrauma 26: 1977-1986.

CysLTs are also implicated in cardiovascular events and diseases. For example, levels of urinary LTE4 are elevated in patients with sleep apnea and acute coronary syndromes, and inhibition of cys-LT signaling by treatment with montelukast during acute hypoxic stress reduced myocardial hypoxic areas in Apoe−/− mice to levels observed under normoxic conditions. Nobili, et al. (2012), PLoS One 7:e41786.

The endothelial barrier strictly maintains vascular and tissue homeostasis, and therefore vascular permeability modulates many physiological processes such as angiogenesis, immune responses, and dynamic exchanges throughout organs. CysLTs, acting through CysLT1 receptors, play an important role in mediating increased vascular permeability in models of both innate and adaptive immunity. Kanaoka and Boyce (2004), J Immunol 173:1503-1510. The endothelial barrier strictly maintains vascular and tissue homeostasis, and therefore vascular permeability modulates many physiological processes such as angiogenesis, immune responses, and dynamic exchanges throughout organs. Azzi et al., (2013) Front. Oncol., 3:1-14, article 211. Thus inhibitors of cysLT(s) are believed to be useful in diseases characterized by aberrant vascular permeability, including but not limited to inflammatory and allergic conditions.

### Asthma and Aspirin-Exacerbated Respiratory Disease (AERD)

The cysLTs are potent lipid mediators that have been shown to induce airway inflammation and have been implicated in the pathogenesis of asthma, particularly aspirin-exacerbated respiratory disease (AERD), also known as aspirin-intolerant asthma (ATA), which is a distinctive asthma phenotype. It is a clinical syndrome associated with chronic severe inflammation in the upper and lower airways resulting in chronic rhinitis, sinusitis, recurrent polyposis, and asthma. AERD generally develops secondary to abnormalities in inflammatory mediators and arachidonic acid biosynthesis expression. Upper and lower airway eosinophil infiltration is a key feature of AERD; however, the exact mechanisms of such chronic eosinophilic inflammation are not fully understood. CysLT over-production may be a key factor in the induction of eosinophilic activation. Leukotiene E4 (LTE4), the most abundant metabolite of the cysLTs, is a potent inducer/amplifier of pulmonary eosinophil recruitment. Palikhe, et al. (2009). Clinical management of AERD symptoms is challenging. AERD patients may present more severe asthma phenotypes with irreversible airflow obstruction and frequent exacerbation of symptoms compared to patients with aspirin-tolerant asthma (ATA). In addition, aspirin ingestion may result in significant morbidity and mortality, and patients must be advised regarding aspirin risk.

Leukotriene receptor antagonists are useful in long-term AERD management and rhinosinusitis. Aspirin desensitization may be required for the relief of upper and lower airway symptoms in AERD patients. Increased cysLTs are potent pro-inflammatory mediators and bronchoconstrictors in AERD pathogenesis. Elevation of Cys-LT levels in the urine, sputum, peripheral blood, and exhaled breath were previously observed after aspirin challenges in AERD patients. Hamad, et al. (2004), Drugs 64:2417-2432 (abstract only, cited in Palikhe, supra). AERD patients had higher exhaled nitric oxide levels and higher baseline levels of CysLTs, particularly LTE4, in saliva, sputum, blood ex vivo and urine than subjects with AERD. Gaber, et al. (2008), Thorax 63:1076-1082.

Animal models of respiratory diseases such as asthma are widely used. Both acute and chronic allergen challenge models are known. For example, see Nials and Uddin (2008) Dis Model Mech. 1: 213-220. The ovalbumin model of induced asthma is commonly used and may be modified to provide a model of acute asthma [e.g., Wu, et al. (2003), Clin & Exp Allergy 33:359-366] or of chronic asthma [e.g., Temelkovski, et al. (1998), Thorax 53: 849-856. Mouse models of airway inflammation induced by natural allergens such as house dust mite and cockroach extracts have also been developed. Johnson, et al. (2004), Am J Respir Crit. Care Med. 169: 378-385; Sarpong, et al. (2003), Int Arch Allergy Immunol 132, 346-354. Others have demonstrated that mice lacking a critical terminal synthetic enzyme, microsomal PGE2 synthase (mPGES)-1 (ptges −/− mice or PGE2 synthase-1 null mice) develop a remarkably AERD-like phenotype in a model of eosinophilic pulmonary inflammation and aspirin-challenged PGE2 synthase-1 null mice reportedly exhibited sustained increases in airway resistance, along with lung mast cell (MC) activation and cysLT overproduction. Liu, et al., (2013), Proc Natl Acad Sci USA. 110:16987-92

Antibody treatment for asthma is known. Omalizumab (XOLAIR, Genentech) is a recombinant humanized IgG1 monoclonal anti-IgE antibody that binds to circulating IgE, regardless of allergen specificity. Proof-of-concept studies have shown that omalizumab reduces both early- and late-phase asthmatic responses after allergen inhalation challenge. Strunk and Bloomberg (2006), N Engl J Med 354:2689-2695. Omalizumab is indicated for adults and adolescents with moderate to severe persistent asthma who have a positive skin test or in vitro reactivity to a perennial aeroallergen and whose symptoms are inadequately controlled with inhaled corticosteroids.

Antibodies to cysLTs are known. For example, a cysteinyl leukotriene ELISA kit is available from antibodies-online, Inc., Atlanta Ga. (catalog no. ABIN930368), as are cysteinyl leukotriene ELISA kits specific for human (cat. no ABIN626393), rat, mouse, guinea pig, rabbit and other cysLTs. An ELISA kit said to have sensitivity and specificity for detection of human LTE4 is also available from the same source (cat. no. ABIN366715), and LTE4 ELISA kits (catalog nos. MBS161552, MBS722103 and MBS703833; no specificity data for binding to other cysLTs are provided for any of the foregoing) are available from MyBioSource Inc., San Diego Calif. A separate ELISA kit for human LTD4 is listed as available from the same source (cat. no. MBS260801); no specificity data is provided for crossreactivity to other cysLTs.

A cysteinyl leukotriene EIA (enzymatic immunoassay) kit using a proprietary monoclonal antibody can be purchased from Cayman Chemical, Ann Arbor Mich. (Item Number 500390). The cysteinyl leukotriene EIA monoclonal antibody alone is also available (Cayman Chemical Item Number 500390). This antibody is listed as having relative specificities of 100% for LTC4 and LTD4, 79% for LTE4 and under 4% for 5,6-diHETE, LTB4, 5(S)-HETE, and arachidonic acid. A monoclonal antibody (mAbLTC) against LTC4 has been described. The antibody is said to show cross-reactivities of 5.4% and 0.5% to LTD4 and LTE4, respectively, and no reactivity with other eicosanoids tested. The authors suggested that the antibody recognized the glutamate residue of the glutathione moiety in LTC4. Kawakami, et al. (2010), BBRC 392: 421-425. A single-chain variable fragment (scFvLTC) comprising variable regions of this antibody was prepared and its affinity and binding specificity with the complete monoclonal antibody. ScFvLTC showed a high affinity for LTC4 comparable to the monoclonal parent antibody, and bound LTD4 and LTE4 with 48% and 17% reactivities, respectively, as compared with LTC4 binding, and almost no affinity for LTB4. Kawakami et al. subsequently showed that mAbLTC and scFvLTC inhibited the binding of LTC4 or LTD4 to CysLT1 receptor (CysLT1R) and CysLT2 receptor (CysLT2R), thus are believed to neutralize the biological activities of LTs by competing their binding to these receptors. Interestingly, mAbLTC also bound cysLT2R antagonists (HAMI3379, BayCysLT2) but not cysLT1R antagonists (pranlukast, MK-571), leading the authors to suggest a structural resemblance of the LT-recognition site of the antibody to those of the receptor. Administration of mAbLTC reduced pulmonary eosinophil infiltration and goblet cell hyperplasia in an ovalbumin (OVA)-induced murine model of asthma. Kawakami et al., (2014) Biochim et Biophys Acta 1840:1625-1633.

A murine monoclonal antibody against sulfidopeptide leukotrienes, called 1A-IDR1, has been described. The mAb reportedly showed a reactivity of 95.7%, 100%, 88.7%, and 89.7% for LTC4, LTD4, LTE4, and N_{ac}-LTE4, respectively. No crossreactivity was reported to have been observed for LTB4, arachidonic acid, or with components of the LT peptide chain such as I-cysteine or glutathione. Reinke, et al. (1991), Biochim et Biophys Acta (Lipids and Lipid Metabolism) 1081:274-278.

Hoppe et al. reported that rabbits immunized against LTE4 or LTC4 conjugated with BSA yielded polyclonal antisera. The antiserum from rabbits immunized against the LTE4 conjugate reportedly had a relative specificity of 46.32%, 12.55%, and 100% for LTC4, LTD4, and LTE4, respectively, and 0.79% for LTB4. Binding to other ligands was reported to be insignificant. The antiserum from rabbits immunized with LTC4 conjugate was said to recognize LTC4 best, with 50% inhibition of binding of label to antibodies by 285 pg), while the relative cross-reaction of LTD4 and LTE4 was reportedly 21.7% and 2.3%, respectively. Hoppe, et al. (1986), FEBS Lett 208:26-30.

Westcott et al. [(2007) Anal Biochem 248, 202-210] reported using a mouse monoclonal antibody purchased from PerSeptive BioResearch Products (Cambridge, Mass.), which was described as having significant cross-reactivity to LTC4 (55%), LTD4 (100%), LTE4 (51%), and N-acetyl LTE4.

Applicant has provided methods and compositions for treating diseases and conditions associated with or characterized by aberrant levels of one or more cysLTs. These methods use antibodies, including monoclonal antibodies and humanized monoclonal antibodies, which bind to and reduce the effective concentration of (neutralize) one or more cysLTs. While cysLT receptor antagonists are employed therapeutically, it is believed that direct interference with the cysLT(s) is advantageous over a receptor-based approach, because it is believed that neutralizing all cysLTs will silence all of the cysLT receptors. As described above, the cysLT receptor antagonists have different specificities, and typically target only one cysLT receptor (CysLT1 in the case of montelukast and other commonly used cysLT receptor antagonists). This leaves other receptors for LTEs free to signal and possibly even become dominant. In addition, as discussed supra, a receptor for LTE4 has not been identified so at this point regulation of LTE4 via receptor agonists is not achievable.

### SUMMARY OF THE INVENTION

The object of the invention concerns methods and compositions for treating a disease or condition associated with aberrant levels of one or more cysLTs. Such methods typically involve administering to a subject, such as a human subject, having such a disease or condition an effective amount of an antibody or antigen-binding antibody fragment that binds one or more cysLTs in order to effect treatment.

Herein provided are methods of treating a disease or condition associated with aberrant levels of one or more cysLTs comprising administering to a subject having said disease or condition an effective amount of an antibody or fragment thereof that binds one or more cysLTs. The disease or condition may be, e.g., an inflammatory disease, allergy, a cardiovascular disease or condition, a disease or condition characterized by aberrant vascular permeability, a central nervous system disease or condition, cancer, a skin condition, a gastrointestinal condition, rheumatoid arthritis, or a respiratory disease or condition, including asthma, aspirin-exacerbated respiratory disease (AERD), airway hyperresponsiveness, and allergic rhinitis.

Antibodies and antigen-binding antibody fragments that reduce the effective concentration of one or more cysLTs are believed to be useful in methods for interfering with disease and conditions correlated with abnormal levels of these cysLTs, such as those listed above. In particular, antibodies (and antigen-binding antibody fragments) that bind one or more of the cysLTs are believed to be useful in treating allergic and inflammatory diseases and conditions, including respiratory diseases and conditions such as asthma, including AERD. In some embodiments, the antibodies to cysLTs are monoclonal antibodies. In some embodiments, the antibodies bind preferentially to one or more cysLTs; in other embodiments, the antibodies are pan-cysLT antibodies that bind to LTC4, LTD4 and LTE4. Where the antibody or fragment thereof binds more than one cysLT, is not necessary for the antibody to bind the cysLTs equally in order to be useful. In another embodiment, the antibodies (or antigen-binding antibody fragments) may be humanized.

As described above, also provided are methods of decreasing inflammation, including inflammation affecting the airway, in a subject comprising administering to the subject an effective amount of an antibody or fragment thereof that binds one or more cysLTs.

Further provided are isolated antibodies or cysLT-binding fragments thereof, pharmaceutical compositions comprising them, and ELISA kits utilizing them. Also provided are compositions that may be used as immunogens or reagents.

The foregoing and other aspects of the invention will become more apparent from the following detailed description and the claims. Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

### BRIEF DESCRIPTION OF THE DRAWINGS

This patent application contains at least one drawing executed in color. Copies of this patent or patent application publication with color drawing(s) will be provided by the Office upon request and payment of the necessary fee.

FIGS. 1A-1C are a three-part series of line graphs showing results of direct ELISA screening of serum samples from three mice for the presence of anti-LTE4 antibodies. Mice were previously immunized with a BS3-facilitated conjugate of LTE4 and BCP. Results shown in FIG. 1A are from mouse F4; results shown in FIG. 1B are from mouse E2; and results shown in FIG. 1C are from mouse F3.

FIGS. 2A-2E are a five-part series of line graphs showing results of direct ELISA screening for the presence of anti-LTE4 antibodies in culture supernatants from five hybridomas prepared from spleens of mice showing high antibody titers. Results shown in FIG. 2A are from hybridoma 9B12; results shown in FIG. 2B are from hybridoma 2G9; results shown in FIG. 2C are from hybridoma 10G4; results shown in FIG. 2D are from hybridoma 14H3; and results shown in FIG. 2E are from hybridoma 2F9.

FIGS. 3A-3B are a two-part series of line graphs showing results of competition ELISAs to determine the specificity of monoclonal antibodies 9B12 (FIG. 3A) and 10G4 (FIG. 3B) for LTC4, LTD4, LTE4, LTB4, 14,15-LTE4; and 5S-HETE.

FIG. 4 is a bar graph showing the preliminary results of a vascular permeability study in mice comparing vehicle (1% DMSO), negative control antibody LT1017 plus LTC4, anti-cysLT monoclonal antibody 9B12 plus LTC4, and anti-cysLT monoclonal antibody 10G4 plus LTC4.

FIG. 5 is a scatter plot showing the effects of murine anti-cysLT antibody 2G9 on vascular permeability in mice, comparing saline alone, LTC4 preincubated with nonspecific control (NS) antibody at a ratio of 1:1 and LTC4 preincubated with anti-cysLT antibody 2G9 (ratio of 1:1 or 1:5). The anti-cysLT antibody neutralized the effect of LTC4 on vascular permeability (as measured by dye extravasation).

FIG. 6 is a scatter plot showing the effects of murine anti-cysLT antibody 10G4 on vascular permeability, comparing mice given saline alone, mice pretreated with subcutaneous injection of 10G4 antibody or nonspecific control antibody (NS) 24 hr prior to LTC4 treatment, and mice injected intraperitoneally with LTC4 preincubated with anti-cysLT antibody 10G4 (ratio of 1:1). The anti-cysLT antibody neutralized the effect of LTC4 on vascular permeability (as measured by dye extravasation) even when given 24 hr in advance. Both IP and SC routes of administration were effective.

FIG. 7 is a line graph showing pharmacokinetics of murine anti-cysLT monoclonal antibodies 9B12 (red) and 10G4 (green) in mouse plasma over time, after intravenous (i.v.) administration.

FIG. 8 is a line graph showing pharmacokinetics of murine anti-cysLT monoclonal antibodies 2G9 (blue) and 10G4 (green) in mouse plasma over time, after intraperitoneal (i.p.) administration.

FIG. 9 is a line graph showing direct LTE4-binding ELISA data for humanized 10G4 variants without (LC, O12-0; HC, 4-59.0) or with full set of backmutations (LC, O12.6; HC, 4-59.6) in the framework region.

FIG. 10 is a line graph showing direct LTE4-binding ELISA of humanized 10G4 antibody variants, each with a single light chain backmutation and no heavy chain backmutations (heavy chain variant 4-59.0).

FIG. 11 is a line graph showing direct LTE4-binding ELISA of humanized 10G4 antibody variants, each with a single light chain backmutation and six heavy chain backmutations (heavy chain variant 4-59.6)

FIG. 12 is a line graph showing direct LTE4-binding ELISA of humanized 10G4 antibody variants, each with a single heavy chain backmutation and four light chain backmutations (light chain variant O12.5).

FIG. 13 is a line graph showing direct LTE4-binding ELISA of humanized 10G4 variants, each with a single heavy chain backmutation and the O12.1 light chain (single backmutation).

FIG. 14 is a line graph showing direct LTE4-binding ELISA of humanized 10G4 variants, each with a single heavy chain backmutation and the O12.2 light chain (single backmutation).

FIG. 15 is a line graph showing direct LTE4-binding ELISA of humanized 10G4 variants, each with a single heavy chain backmutation and the O12.3 light chain (single backmutation).

FIG. 16 is a line graph showing direct LTE4-binding ELISA of humanized 10G4 variants, each with a single heavy chain backmutation and the O12.4 light chain (single backmutation).

FIG. 17 is a line graph showing the DAI (disease activity index) of mice with DSS-induced colitis after treatment with vehicle, positive control (Cyclosporin A or CsA), murine anti cysLT antibodies 10G4 and 2G9, or nonspecific antibody control. Vehicle and nonspecific antibody (LT1014) treated groups had the highest DAI, and anti-cysLT antibody 10G4 and positive control cyclosporine A (CsA)-treated animals had the lowest DAI. Statistical significance for 10G4 vs vehicle is shown (* P<0.05, ** P<0.01, *** P<0.001) based on multiple t-tests.

FIG. 18 is a line graph showing airway hyperresponsiveness (measured as percent of baseline “enhanced pause” or “penh”) in mice with ovalbumin (OVA)-induced acute asthma. As expected, the mice in which asthma was induced showed the highest penh and mice given no OVA showed the lowest penh. Anti cysLT antibody 10G4 lowered the penh to roughly that of the positive control, dexamethasone. Antibody 9B12 lowered the penh to an intermediate level.

FIG. 19 is a series of bar graphs showing total numbers of cells in bronchoalveolar lavage (BAL) fluid from mice with OVA-induced acute asthma after treatment with LT1017 (nonspecific control antibody), anti-cysLT antibody 9B12 or anti-cysLT antibody 10G4.

### DETAILED DESCRIPTION OF THE INVENTION

### Definitions

In addition to the terms defined in this section, others are defined elsewhere in the specification, as necessary. Unless otherwise expressly defined herein, terms of art used in this specification will have their art-recognized meanings.

The term “cysLT” is an abbreviation for cysteinyl leukotriene. Physiologically important cysLTs include leukotriene C4 (LTC4), leukotriene D4 (LTD4) and leukotriene E4 (LTE4). In the context of this invention, “cysLT” or “cysLTs” means cysteinyl leukotrienes that occur in nature and are correlated or associated with, or implicated in, a disease or other unhealthful condition. Preferred cysLTs are LTC4, LTD4, and LTE4.

The term “aberrant” means excessive or unwanted, for example in reference to levels or effective concentrations of a cellular target such as a protein or bioactive lipid.

The term “antibody” (“Ab”) or “immunoglobulin” (Ig) refers to any form of a peptide, polypeptide derived from, modeled after or encoded by, an immunoglobulin gene, or fragment thereof, which is capable of binding an antigen or epitope. See, e.g., IMMUNOBIOLOGY, Fifth Edition, Janeway, et al., ed. Garland Publishing (2001). The term “antibody” is used herein in the broadest sense, and encompasses monoclonal, polyclonal or multispecific antibodies, minibodies, heteroconjugates, diabodies, triabodies, chimeric, antibodies, synthetic antibodies, antibody fragments that retain antigen binding activity, and binding agents that employ the complementarity determining regions (CDRs) of a parent antibody. Antibodies are defined herein as retaining at least one desired activity of the parent antibody. Desired activities may include the ability to bind the antigen, the ability to bind the antigen preferentially, and the ability to alter cytokine profile(s) in vitro.

Native antibodies (native immunoglobulins) are usually heterotetrameric glycoproteins of about 150,000 Daltons, typically composed of two identical light (L) chains and two identical heavy (H) chains. The heavy chain is approximately 50 kD in size, and the light chain is approximately 25 kDa. Each light chain is typically linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end. The constant domain of the light chain is aligned with the first constant domain of the heavy chain, and the light-chain variable domain is aligned with the variable domain of the heavy chain. Particular amino acid residues are believed to form an interface between the light- and heavy-chain variable domains.

The light chains of antibodies (immunoglobulins) from any vertebrate species can be assigned to one of two clearly distinct types, called kappa (κ) and lambda (λ), based on the amino acid sequences of their constant domains. The ratio of the two types of light chain varies from species to species. As a way of example, the average κ to λ ratio is 20:1 in mice, whereas in humans it is 2:1 and in cattle it is 1:20.

Depending on the amino acid sequence of the constant domain of their heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA, and IgA2. The heavy-chain constant domains that correspond to the different classes of immunoglobulins are called alpha, delta, epsilon, gamma, and mu, respectively. The subunit structures and three-dimensional configurations of different classes of immunoglobulins are well known.

An antibody may be designed and/or prepared from the amino acid sequence of another antibody (often referred to as the “parent” or “native” antibody) that is directed to the same antigen by virtue of addition, deletion and/or substitution of one or more amino acid residue(s) in the antibody sequence and which retains at least one desired activity of the parent antibody. Desired activities can include the ability to bind the antigen specifically, the ability to inhibit proliferation in vitro, the ability to inhibit angiogenesis in vivo, and the ability to alter cytokine profile in vitro. The amino acid change(s) may be within a variable region or a constant region of a light chain and/or a heavy chain, including in the Fc region, the Fab region, the CH1 domain, the CH2 domain, the CH3 domain, and the hinge region. In one embodiment one or more amino acid substitution(s) are made in one or more hypervariable region(s) of the parent antibody. For example, there may be at least one, e.g. from about one to about ten, and preferably from about two to about five, substitutions in one or more hypervariable regions compared to the parent antibody. Ordinarily, amino acid changes will result in a new antibody amino acid sequence having at least 50% amino acid sequence identity with the parent antibody heavy or light chain variable domain sequences, more preferably at least 65%, more preferably at 80%, more preferably at least 85%, more preferably at least 90%, and most preferably at least 95%. Identity or homology with respect to this sequence is defined herein as the percentage of amino acid residues in the candidate sequence that are identical with the parent antibody residues, after aligning the sequences and introducing gaps, if necessary, to achieve the maximum percent sequence identity. None of N-terminal, C-terminal, or internal extensions, deletions, or insertions into the antibody sequence shall be construed as affecting sequence identity or homology.

As used herein, “antibody fragment” refers to a portion of an intact antibody that includes the antigen binding site(s) or variable regions of an intact antibody, wherein the portion can be free of the constant heavy chain domains (e.g., CH2, CH3, and CH4) of the Fc region of the intact antibody. Alternatively, portions of the constant heavy chain domains (e.g., CH2, CH3, and CH4) can be included in the “antibody fragment”. Antibody fragments retain antigen-binding ability and include Fab, Fab′, F(ab′)2, Fd, and Fv fragments; diabodies; triabodies; single-chain antibody molecules (sc-Fv); minibodies, nanobodies, and multispecific antibodies formed from antibody fragments. Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)2 fragment that has two antigen-combining sites and is still capable of cross-linking antigen. By way of example, a Fab fragment also contains the constant domain of a light chain and the first constant domain (CH1) of a heavy chain. “Fv” is the minimum antibody fragment that contains a complete antigen-recognition and -binding site. This region consists of a dimer of one heavy chain and one light chain variable domain in tight, non-covalent association. It is in this configuration that the three hypervariable regions (or complementarity determining regions or “CDRs”) of each variable domain interact to define an antigen-binding site on the surface of the VH-VL dimer. Collectively, the six hypervariable regions confer antigen-binding specificity to the antibody. However, even a single variable domain (or half of an Fv comprising only three hypervariable regions specific for an antigen) has the ability to recognize and bind antigen, although at a lower affinity than the entire binding site. “Single-chain Fv” or “sFv” antibody fragments comprise the VH and VL domains of antibody, wherein these domains are present in a single polypeptide chain. Generally, the Fv polypeptide further comprises a polypeptide linker between the VH and VL domains that enables the sFv to form the desired structure for antigen binding. For a review of sFv, see Pluckthun in The Pharmacology of Monoclonal Antibodies, vol. 113, Rosenburg and Moore eds. Springer-Verlag, New York, pp. 269-315 (1994).

The Fab fragment also contains the constant domain of the light chain and the first constant domain (CH1) of the heavy chain. Fab′ fragments differ from Fab fragments by the addition of a few residues at the carboxyl terminus of the heavy chain CH1 domain including one or more cysteine(s) from the antibody hinge region. Fab′-SH is the designation herein for Fab′ in which the cysteine residue(s) of the constant domains bear a free thiol group. F(ab′)2 antibody fragments originally were produced as pairs of Fab′ fragments which have hinge cysteines between them. Other chemical couplings of antibody fragments are also known.

An “anti-cysLT antibody” or an “immune-derived moiety reactive against cysLT” refers to any antibody or antibody-derived molecule that binds one or more of the cysLTs, preferably one or more of LTC4, LTD4, and LTE4. As will be understood from these definitions, antibodies or immune-derived moieties may be polyclonal or monoclonal and may be generated through a variety of means, and/or may be isolated from an animal, including a human subject.

A “bioactive lipid” refers to a lipid signaling molecule. Bioactive lipids are distinguished from structural lipids (e.g., membrane-bound phospholipids) in that they mediate extracellular and/or intracellular signaling and thus are involved in controlling the function of many types of cells by modulating differentiation, migration, proliferation, secretion, survival, and other processes. In vivo, bioactive lipids can be found in extracellular fluids, where they can be complexed with other molecules, for example serum proteins such as albumin and lipoproteins, or in “free” form, i.e., not complexed with another molecule species. As extracellular mediators, some bioactive lipids alter cell signaling by activating membrane-bound ion channels or GPCRs or enzymes or factors that, in turn, activate complex signaling systems that result in changes in cell function or survival. As intracellular mediators, bioactive lipids can exert their actions by directly interacting with intracellular components such as enzymes, ion channels or structural elements such as actin.

Examples of bioactive lipids include sphingolipids such as ceramide, ceramide-1-phosphate (C1P), sphingosine, sphinganine, sphingosylphosphorylcholine (SPC) and sphingosine-1-phosphate (S1P). Sphingolipids and their derivatives and metabolites are characterized by a sphingoid backbone (derived from sphingomyelin). Sphingolipids and their derivatives and metabolites represent a group of extracellular and intracellular signaling molecules with pleiotropic effects on important cellular processes. They include sulfatides, gangliosides and cerebrosides. Other bioactive lipids are characterized by a glycerol-based backbone; for example, lysophospholipids such as lysophosphatidyl choline (LPC) and various lysophosphatidic acids (LPA), as well as phosphatidylinositol (PI), phosphatidylethanolamine (PEA), phosphatidic acid, platelet activating factor (PAF), cardiolipin, phosphatidylglycerol (PG) and diacylglyceride (DG). Yet other bioactive lipids are derived from arachidonic acid; these include the eicosanoids and eicosanoid metabolites such as the HETEs, cannabinoids, leukotrienes, prostaglandins, lipoxins, epoxyeicosatrienoic acids, and isoeicosanoids, and non-eicosanoid cannabinoid mediators. Other bioactive lipids, including other phospholipids and their derivatives, may also be used.

Specifically excluded from the class of bioactive lipids as defined herein are lipids such as phosphatidylcholine, phosphatidylserine, and metabolites and derivatives thereof that function primarily as structural members of the inner and/or outer leaflet of cellular membranes.

The term “biologically active,” in the context of an antibody or antibody fragment, refers to an antibody or antibody fragment that is capable of binding the desired epitope and in some ways exerting a biologic effect. Biological effects include, but are not limited to, the modulation of a growth signal, the modulation of an anti-apoptotic signal, the modulation of an apoptotic signal, the modulation of the effector function cascade, and modulation of other ligand interactions.

A “biomarker” is a specific biochemical in the body that has a particular molecular feature that makes it useful for measuring the progress of disease or the effects of treatment. For example, S1P is a biomarker for certain hyperproliferative and/or cardiovascular conditions.

A “carrier” refers to a moiety adapted for conjugation to a hapten, thereby rendering the hapten immunogenic. A representative, non-limiting class of carriers is proteins, examples of which include albumin, keyhole limpet hemocyanin, hemaglutanin, tetanus, and diptheria toxoid. Other suitable classes and examples of carriers are known in the art. These, as well as later discovered or invented naturally occurring or synthetic carriers, can be adapted for application in accordance with the disclosure herein.

As used herein, the expressions “cell,” “cell line,” and “cell culture” are used interchangeably and all such designations include progeny. Thus, the words “transformants” and “transformed cells” include the primary subject cell and cultures derived there from without regard for the number of transfers. It is also understood that all progeny may not be precisely identical in DNA content, due to deliberate or inadvertent mutations. Mutant progeny that have the same function or biological activity as screened for in the originally transformed cell are included. Where distinct designations are intended, it will be clear from the context.

The term “combination therapy” refers to a therapeutic regimen that involves the provision of at least two distinct therapies to achieve an indicated therapeutic effect. For example, a combination therapy may involve the administration of two or more chemically distinct active ingredients, for example, a fast-acting corticosteroid agent and an anti-lipid antibody, or two different antibodies. Alternatively, a combination therapy may involve the administration of an anti-lipid antibody together with the delivery of another treatment, such as radiation therapy and/or surgery. Further, a combination therapy may involve administration of an anti-lipid antibody together with one or more other biological agents (e.g., corticosteroid), antiinflammatory agents and/or another treatment such as radiation and/or surgery. In the context of the administration of two or more chemically distinct active ingredients, it is understood that the active ingredients may be administered as part of the same composition or as different compositions. When administered as separate compositions, the compositions comprising the different active ingredients may be administered at the same or different times, by the same or different routes, using the same of different dosing regimens, all as the particular context requires and as determined by the attending physician. Similarly, when one or more anti-lipid antibody species, alone or in conjunction with one or more chemotherapeutic agents are combined with, for example, radiation and/or surgery, the drug(s) may be delivered before or after surgery or radiation treatment.

The term “constant domain” refers to the C-terminal region of an antibody heavy or light chain. Generally, the constant domains are not directly involved in the binding properties of an antibody molecule to an antigen, but exhibit various effector functions, such as participation of the antibody in antibody-dependent cellular toxicity. Here, “effector functions” refer to the different physiological effects of antibodies (e.g., opsonization, cell lysis, mast cell, basophil and eosinophil degranulation, and other processes) mediated by the recruitment of immune cells by the molecular interaction between the Fc domain and proteins of the immune system. The isotype of the heavy chain determines the functional properties of the antibody. Their distinctive functional properties are conferred by the carboxy-terminal portions of the heavy chains, where they are not associated with light chains.

A “derivatized bioactive lipid” is a bioactive lipid, e.g., a cysLT, which is derivatized with a reactive group (e.g., a sulfhydryl (thiol) group, a carboxylic acid group, a cyano group, an ester, a hydroxy group, an alkene, an alkyne, an acid chloride group or a halogen atom) that serves to activate the bioactive lipid for reaction with a molecule, e.g., for conjugation to a carrier. Preferably the reactive group is positioned to allow the epitope to be accessible (i.e., not hindered by the reactive group), and in some embodiments the reactive group is positioned at the end of a flexible “tail” on the lipid, such as a hydrocarbon chain, which may be part of the native lipid or may be added for purposes of derivatization. For example, in the case of sphingosine-1-phosphate, a thiol group was positioned at the omega carbon (terminus) of the hydrocarbon chain of the molecule, allowing the polar head group to be accessible as an epitope. See, for example. U.S. Pat. No. 8,067,549, which is commonly assigned with the instant invention.

A “bioactive lipid conjugate” refers to a bioactive lipid that is covalently conjugated to a carrier. The lipid may be derivatized as described above in order to be reactive for conjugation, or the native lipid may contain a reactive group that may be used to conjugate the lipid to a carrier. The carrier may be a protein molecule or may be a nonproteinaceous moiety such as polyethylene glycol, colloidal gold, adjuvants or silicone beads. A bioactive lipid conjugate may be used as an immunogen for generating an antibody response according to the instant disclosure, and the same or a different bioactive lipid conjugate may be used as a detection reagent for detecting the antibody thus produced. In some embodiments the derivatized bioactive lipid conjugate is attached to a solid support when used for detection.

“Effective concentration” refers to the absolute, relative, and/or available concentration and/or activity, for example of certain undesired bioactive lipids. In other words, the effective concentration of a bioactive lipid is the amount of lipid available, and able, to perform its biological function. In the present disclosure, an immune-derived moiety such as, for example, a monoclonal antibody directed to a bioactive lipid is able to reduce the effective concentration of the lipid by binding to the lipid and rendering it unable to perform its biological function. In this example, the lipid itself is still present (it is not degraded by the antibody, in other words) but can no longer bind its receptor or other targets to cause a downstream effect, so “effective concentration” rather than absolute concentration is the appropriate measurement. Methods and assays exist for directly and/or indirectly measuring the effective concentration of bioactive lipids.

An “epitope” or “antigenic determinant” refers to that portion of an antigen that reacts with an antibody antigen-binding portion derived from an antibody.

A “fully human antibody” can refer to an antibody produced in a genetically engineered (i.e., transgenic) mouse (e.g., HUMAB-MOUSE from Medarex Inc., Princeton N.J.) that, when presented with an immunogen, can produce a human antibody that does not necessarily require CDR grafting. These antibodies are fully human (100% human protein sequences) from animals such as mice in which the non-human antibody genes are suppressed and replaced with human antibody gene expression. The applicants believe that antibodies could be generated against bioactive lipids when presented to these genetically engineered mice or other animals that might be able to produce human frameworks for the relevant CDRs.

A “hapten” is a substance that is non-immunogenic but can react with an antibody or antigen-binding portion derived from an antibody. In other words, haptens have the property of antigenicity but not immunogenicity. A hapten is generally a small molecule that can, under most circumstances, elicit an immune response (i.e., act as an antigen) only when attached to a carrier, for example, a protein, polyethylene glycol (PEG), colloidal gold, silicone beads, or the like. The carrier may be one that also does not elicit an immune response by itself. A representative, non-limiting class of hapten molecules is proteins, examples of which include albumin, keyhole limpet hemocyanin, hemaglutanin, tetanus, and diphtheria toxoid. Other classes and examples of hapten molecules are known in the art. These, as well as later discovered or invented naturally occurring or synthetic haptens, can be adapted for use according to this disclosure.

The term “heteroconjugate antibody” can refer to two covalently joined antibodies. Such antibodies can be prepared using known methods in synthetic protein chemistry, including using crosslinking agents. As used herein, the term “conjugate” refers to molecules formed by the covalent attachment of one or more antibody fragment(s) or binding moieties to one or more polymer molecule(s).

“Humanized” forms of non-human (e.g., murine) antibodies are chimeric antibodies that contain minimal sequence derived from non-human immunoglobulin. Or, looked at another way, a humanized antibody is a human antibody that also contains selected sequences from non-human (e.g., murine) antibodies in place of the human sequences. A humanized antibody can include conservative amino acid substitutions or non-natural residues from the same or different species that do not significantly alter its binding and/or biologic activity. Such antibodies are chimeric antibodies that contain minimal sequence derived from non-human immunoglobulins. For the most part, humanized antibodies are human immunoglobulins (recipient antibody) in which residues from a complementary-determining region (CDR) of the recipient are replaced by residues from a CDR of a non-human species (donor antibody) such as mouse, rat, camel, bovine, goat, or rabbit having the desired properties. In some instances, framework region (FR) residues of the human immunoglobulin are replaced by corresponding residues from the non-human parent antibody (each replacement being called a “backmutation”).

Furthermore, humanized antibodies can comprise residues that are found neither in the recipient antibody nor in the imported CDR or framework sequences. These modifications are made to further refine and maximize antibody performance. Thus, in general, a humanized antibody will comprise all of at least one, and in one aspect two, variable domains, in which all or all of the hypervariable loops correspond to those of a non-human immunoglobulin and all or substantially all of the framework regions are those of a human immunoglobulin sequence. The humanized antibody optionally also will comprise at least a portion of an immunoglobulin constant region (Fc), or that of a human immunoglobulin. See, e.g., Cabilly, et al., U.S. Pat. No. 4,816,567; Cabilly, et al., European Patent No. 0,125,023 B1; Boss, et al., U.S. Pat. No. 4,816,397; Boss, et al., European Patent No. 0,120,694 B1; Neuberger, et al., WO 86/01533; Neuberger, et al., European Patent No. 0,194,276 B1; Winter, U.S. Pat. No. 5,225,539; Winter, European Patent No. 0,239,400 B1; Padlan, et al., European Patent Application No. 0,519,596 A1; Queen, et al. (1989), Proc. Nat'l Acad. Sci. USA, vol. 86:10029-10033). For further details, see Jones et al., Nature 321:522-525 (1986); Reichmann et al., Nature 332:323-329 (1988); and Presta, Curr Op Struct Biol 2:593-596 (1992) and Hansen, WO2006105062. Humanized antibodies may be preferred to nonhuman antibodies for use in humans because the human body may mount an immune response against the nonhuman antibodies that are viewed as a foreign substance. A human anti-mouse antibody (HAMA) response has been observed in a significant fraction of patients given mouse antibody therapy.

An “immune-derived moiety” includes any antibody (Ab) or immunoglobulin (Ig), and refers to any form of a peptide, polypeptide derived from, modeled after or encoded by, an immunoglobulin gene, or a fragment of such peptide or polypeptide that is capable of binding an antigen or epitope (see, e.g., Immunobiology, 5th Edition, Janeway, Travers, Walport, Shlomchiked. (editors), Garland Publishing (2001)). In the present disclosure, the antigen is a lipid molecule, such as a bioactive lipid molecule.

An “immunogen” is a molecule capable of inducing a specific immune response, particularly an antibody response in an animal to whom the immunogen has been administered. In the instant disclosure, the immunogen is a derivatized bioactive lipid conjugated to a carrier, i.e., a “derivatized bioactive lipid conjugate”. The derivatized bioactive lipid conjugate used as the immunogen may be used as capture material for detection of the antibody generated in response to the immunogen. Thus the immunogen may also be used as a detection reagent. Alternatively, the derivatized bioactive lipid conjugate used as capture material may have a different linker and/or carrier moiety from that in the immunogen.

To “inhibit,” particularly in the context of a biological phenomenon, means to decrease, reduce, suppress or delay. For example, a treatment yielding “inhibition of inflammation” may mean that inflammation does not occur, or occurs more slowly or to a lesser extent, than in the untreated control.

An “isolated” antibody is one that has been identified and separated and/or recovered from a component of its natural environment. Contaminant components of its natural environment are materials that would interfere with diagnostic or therapeutic uses for the antibody, and may include enzymes, hormones, and other proteinaceous or nonproteinaceous solutes. In preferred embodiments, the antibody will be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

The word “label” when used herein refers to a detectable compound or composition, such as one that is conjugated directly or indirectly to the antibody. The label may itself be detectable by itself (e.g., radioisotope labels or fluorescent labels) or, in the case of an enzymatic label, may catalyze chemical alteration of a substrate compound or composition that is detectable.

A “ligand” is a substance that is able to bind to and form a complex with a biomolecule to serve a biological purpose. Thus an antigen may be described as a ligand of the antibody to which it binds.

An “isolated” nucleic acid molecule is a nucleic acid molecule that is identified and separated from at least one contaminant nucleic acid molecule with which it is ordinarily associated in the natural source of the antibody nucleic acid. An isolated nucleic acid molecule is other than in the form or setting in which it is found in nature. Isolated nucleic acid molecules therefore are distinguished from the nucleic acid molecule as it exists in natural cells. However, an isolated nucleic acid molecule includes a nucleic acid molecule contained in cells that ordinarily express the antibody where, for example, the nucleic acid molecule is in a chromosomal location different from that of natural cells.

A “liquid composition” refers to one that, in its filled and finished form as provided from a manufacturer to an end user (e.g., a doctor or nurse), is a liquid or solution, as opposed to a solid. Here, “solid” refers to compositions that are not liquids or solutions. For example, solids include dried compositions prepared by lyophilization, freeze-drying, precipitation, and similar procedures.

The expression “linear antibodies” when used throughout this application refers to the antibodies described in Zapata, et al. Protein Eng 8(10):1057-1062 (1995). Briefly, these antibodies comprise a pair of tandem Fd segments (VH—CH1-VH—CH1) that form a pair of antigen binding regions. Linear antibodies can be bispecific or monospecific.

The term “metabolites” refers to compounds from which a given cysLT is made, as well as those that result from the degradation of a cysLT; that is, compounds that are involved in the cysLT metabolic pathways. The term “metabolic precursors” may be used to refer to compounds from which a given cysLT is made.

The term “monoclonal antibody” (mAb) as used herein refers to an antibody obtained from a population of substantially homogeneous antibodies, or to said population of antibodies. The individual antibodies comprising the population are essentially identical, except for possible naturally occurring mutations that may be present in minor amounts. Monoclonal antibodies are highly specific, being directed against a single antigenic site. Furthermore, in contrast to conventional (polyclonal) antibody preparations that typically include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present disclosure may be made by the hybridoma method first described by Kohler, et al., Nature 256:495 (1975), or may be made by recombinant DNA methods (see, e.g., U.S. Pat. No. 4,816,567). The “monoclonal antibodies” may also be isolated from phage antibody libraries using the techniques described in Clackson, et al., Nature (1991), 352:624-628, and Marks, et al. (1991), J Mol Biol 222:581-597, for example, or by other methods known in the art. The monoclonal antibodies herein specifically include chimeric antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison, et al. (1984), Proc Natl Acad Sci USA 81:6851-6855).

“Monotherapy” refers to a treatment regimen based on the delivery of one therapeutically effective compound, whether administered as a single dose or several doses over time.

The term “multispecific antibody” can refer to an antibody, or a monoclonal antibody, having binding properties for at least two different epitopes. In one embodiment, the epitopes are from the same antigen. In another embodiment, the epitopes are from two or more different antigens. Methods for making multispecific antibodies are known in the art. Multispecific antibodies include bispecific antibodies (having binding properties for two epitopes), trispecific antibodies (three epitopes) and so on. For example, multispecific antibodies can be produced recombinantly using the co-expression of two or more immunoglobulin heavy chain/light chain pairs. Alternatively, multispecific antibodies can be prepared using chemical linkage. One of skill can produce multispecific antibodies using these or other methods as may be known in the art. Multispecific antibodies include multispecific antibody fragments. One example of a multispecific (in this case, bispecific) antibody is an antibody having binding properties for an S1P epitope and an LTE4 epitope, which thus is able to recognize and bind to both S1P and LTE4. Another example of a bispecific antibody is an antibody having binding properties for an epitope from a bioactive lipid and an epitope from a cell surface antigen. Thus the antibody is able to recognize and bind the bioactive lipid and is able to recognize and bind to cells, e.g., for targeting purposes.

“Neoplasia” or “cancer” refers to abnormal and uncontrolled cell growth. A “neoplasm”, or tumor or cancer, is an abnormal, unregulated, and disorganized proliferation of cell growth, and is generally referred to as cancer. A neoplasm may be benign or malignant. A neoplasm is malignant, or cancerous, if it has properties of destructive growth, invasiveness, and metastasis. Invasiveness refers to the local spread of a neoplasm by infiltration or destruction of surrounding tissue, typically breaking through the basal laminas that define the boundaries of the tissues, thereby often entering the body's circulatory system. Metastasis typically refers to the dissemination of tumor cells by lymphatics or blood vessels. Metastasis also refers to the migration of tumor cells by direct extension through serous cavities, or subarachnoid or other spaces. Through the process of metastasis, tumor cell migration to other areas of the body establishes neoplasms in areas away from the site of initial appearance.

“Neovascularization” refers to the formation of new blood vessels.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

The term “pharmaceutically acceptable salt” refers to a salt, such as used in formulation, which retains the biological effectiveness and properties of the agents and compounds of this and which are is biologically or otherwise desirable. In many cases, the agents and compounds disclosed herein are capable of forming acid and/or base salts by virtue of the presence of charged groups, for example, charged amino and/or carboxyl groups or groups similar thereto. Pharmaceutically acceptable acid addition salts may be prepared from inorganic and organic acids, while pharmaceutically acceptable base addition salts can be prepared from inorganic and organic bases. For a review of pharmaceutically acceptable salts (see Berge, et al. (1977) J Pharm Sci, vol. 66, 1-19).

A “plurality” means more than one.

The term “promoter” includes all sequences capable of driving transcription of a coding sequence in a cell. Thus, promoters used in the constructs may include cis-acting transcriptional control elements and regulatory sequences that are involved in regulating or modulating the timing and/or rate of transcription of a gene. For example, a promoter can be a cis-acting transcriptional control element, including an enhancer, a promoter, a transcription terminator, an origin of replication, a chromosomal integration sequence, 5′ and 3′ untranslated regions, or an intronic sequence, which are involved in transcriptional regulation. Transcriptional regulatory regions suitable for use include but are not limited to the human cytomegalovirus (CMV) immediate-early enhancer/promoter, the SV40 early enhancer/promoter, the E. coli lac or trp promoters, and other promoters known to control expression of genes in prokaryotic or eukaryotic cells or their viruses.

The term “recombinant DNA” refers to nucleic acids and gene products expressed therefrom that have been engineered, created, or modified by man. “Recombinant” polypeptides or proteins are polypeptides or proteins produced by recombinant DNA techniques, for example, from cells transformed by an exogenous DNA construct encoding the desired polypeptide or protein. “Synthetic” polypeptides or proteins are those prepared by chemical synthesis.

The terms “separated”, “purified”, “isolated”, and the like mean that one or more components of a sample contained in a sample-holding vessel are or have been physically removed from, or diluted in the presence of, one or more other sample components present in the vessel. Sample components that may be removed or diluted during a separating or purifying step include, chemical reaction products, non-reacted chemicals, proteins, carbohydrates, lipids, and unbound molecules.

By “solid phase” is meant a non-aqueous matrix such as one to which the antibody can adhere. Examples of solid phases encompassed herein include those formed partially or entirely of glass (e.g., controlled pore glass), polysaccharides (e.g., agarose), polyacrylamides, polystyrene, polyvinyl alcohol and silicones. In certain embodiments, depending on the context, the solid phase can comprise the well of an assay plate; in others it is a purification column (e.g., an affinity chromatography column). This term also includes a discontinuous solid phase of discrete particles, such as those described in U.S. Pat. No. 4,275,149.

The term “species” is used herein in various contexts, e.g., a particular species of cysteinyl leukotriene (cysLT), for example, LTC4, LTD4, LTE4, and LTF4. In each context, the term refers to a population of chemically indistinct molecules of the sort referred in the particular context.

The term “specific” or “specificity” in the context of antibody-antigen interactions refers to the selective, non-random interaction between an antibody and its target epitope. Here, the term “antigen” refers to a molecule that is recognized and bound by an antibody molecule or other immune-derived moiety. The specific portion of an antigen that is bound by an antibody is termed the “epitope”. This interaction depends on the presence of structural, hydrophobic/hydrophilic, and/or electrostatic features that allow appropriate chemical or molecular interactions between the molecules. Thus, an antibody is commonly said to “bind” (or “specifically bind”) or be “reactive with” (or “specifically reactive with), or, equivalently, “reactive against” (or “specifically reactive against”) the epitope of its target antigen. Antibodies are commonly described in the art as being “against” or “to” their antigens as shorthand for antibody binding to the antigen. Thus an “antibody that binds LTE4”, an “antibody reactive against LTE4,” an “antibody reactive with LTE4,” an “antibody to LTE4” and an “anti-LTE4 antibody” all have the same meaning in the art. Antibody molecules can be tested for specificity of binding by comparing binding to the desired antigen to binding to unrelated antigen or analogue antigen or antigen mixture under a given set of conditions. Preferably, an antibody will lack significant binding to unrelated antigens, and it may be preferred for the antibody to lack specific binding to one or more analogs of the target antigen. “Specifically associate” and “specific association” and the like refer to a specific, non-random interaction between two molecules, which interaction depends on the presence of structural, hydrophobic/hydrophilic, and/or electrostatic features that allow appropriate chemical or molecular interactions between the molecules.

Herein, “stable” refers to an interaction between two molecules (e.g., a peptide and a TLR molecule) that is sufficiently stable such that the molecules can be maintained for the desired purpose or manipulation. For example, a “stable” interaction between a peptide and a TLR molecule refers to one wherein the peptide becomes and remains associated with a TLR molecule for a period sufficient to achieve the desired effect.

A “subject” or “patient” refers to an animal in need of treatment that can be effected by compositions disclosed herein. Animals that can be treated include vertebrates, with mammals such as bovine, canine, equine, feline, ovine, porcine, and primate (including humans and non-human primates) animals being particularly preferred examples.

A “surrogate marker” refers to laboratory measurement of biological activity within the body that indirectly indicates the effect of treatment on disease state. Examples of surrogate markers for hyperproliferative and/or cardiovascular conditions include SPHK and/or S1PRs.

A “therapeutic agent” refers to a drug or compound that is intended to provide a therapeutic effect including, but not limited to: anti-inflammatory drugs including COX inhibitors and other NSAIDS, anti-angiogenic drugs, chemotherapeutic drugs as defined above, cardiovascular agents, immunomodulatory agents, agents that are used to treat neurodegenerative disorders, ophthalmic drugs, anti-fibrotics, etc.

A “therapeutically effective amount” (or “effective amount”) refers to an amount of an active ingredient sufficient to effect treatment when administered to a subject in need of such treatment. Accordingly, what constitutes a therapeutically effective amount of a composition may be readily determined by one of ordinary skill in the art. In the context of cancer therapy, a “therapeutically effective amount” is one that produces an objectively measured change in one or more parameters associated with cancer cell survival or metabolism, including an increase or decrease in the expression of one or more genes correlated with the particular cancer, reduction in tumor burden, cancer cell lysis, the detection of one or more cancer cell death markers in a biological sample (e.g., a biopsy and an aliquot of a bodily fluid such as whole blood, plasma, serum, urine, etc.), induction of induction apoptosis or other cell death pathways, etc. Of course, the therapeutically effective amount will vary depending upon the particular subject and condition being treated, the weight and age of the subject, the severity of the disease condition, the particular compound chosen, the dosing regimen to be followed, timing of administration, the manner of administration and the like, all of which can readily be determined by one of ordinary skill in the art. It will be appreciated that in the context of combination therapy, what constitutes a therapeutically effective amount of a particular active ingredient may differ from what constitutes a therapeutically effective amount of the active ingredient when administered as a monotherapy (i.e., a therapeutic regimen that employs only one chemical entity as the active ingredient). The compositions described herein are used in methods of bioactive lipid-based therapy.

As used herein, the terms “therapy” and “therapeutic” encompasses the full spectrum of prevention and/or treatments for a disease, disorder or physical trauma. A “therapeutic” agent may act in a manner that is prophylactic or preventive, including those that incorporate procedures designed to target individuals that can be identified as being at risk (e.g, via pharmacogenetics); or in a manner that is ameliorative or curative in nature; or may act to slow the rate or extent of the progression of at least one symptom of a disease or disorder being treated; or may act to minimize the time required, the occurrence or extent of any discomfort or pain, or physical limitations associated with recuperation from a disease, disorder, or physical trauma; or may be used as an adjuvant to other therapies and treatments.

The term “treatment” or “treating” means any treatment of a disease or disorder, including preventing or protecting against the disease or disorder (that is, causing the clinical symptoms not to develop); inhibiting the disease or disorder (i.e., arresting, delaying or suppressing the development of clinical symptoms; and/or relieving the disease or disorder (i.e., causing the regression of clinical symptoms). As will be appreciated, it is not always possible to distinguish between “preventing” and “suppressing” a disease or disorder because the ultimate inductive event or events may be unknown or latent. Those “in need of treatment” include those already with the disorder as well as those in which the disorder is to be prevented. Accordingly, the term “prophylaxis” will be understood to constitute a type of “treatment” that encompasses both “preventing” and “suppressing”. The term “protection” thus includes “prophylaxis”.

The term “therapeutic regimen” means any treatment of a disease or disorder using chemotherapeutic and cytotoxic agents, radiation therapy, surgery, gene therapy, DNA vaccines and therapy, siRNA therapy, anti-angiogenic therapy, immunotherapy, bone marrow transplants, aptamers and other biologics such as antibodies and antibody fragments, receptor decoys, and other protein-based therapeutics.

The “variable” region of an antibody comprises framework and complementarity determining regions (CDRs, otherwise known as hypervariable regions). The variability is not evenly distributed throughout the variable domains of antibodies. It is concentrated in six CDR segments, three in each of the light chain and the heavy chain variable domains. The more highly conserved portions of variable domains are called the framework region (FR). The variable domains of native heavy and light chains each comprise four FRs (FR1, FR2, FR3, and FR4, respectively), largely adopting a beta-sheet configuration, connected by three hypervariable regions, which form loops connecting, and in some cases forming part of, the beta-sheet structure. The term “hypervariable region” when used herein refers to the amino acid residues of an antibody which are responsible for antigen binding. The hypervariable region comprises amino acid residues from a “complementarity determining region” or “CDR” (for example, residues 24-34 (L1), 50-56 (L2) and 89-97 (L3) in the light chain variable domain and 31-35 (H1), 50-65 (H2), and 95-102 (H3) in the heavy chain variable domain; Kabat, et al., Sequences of Proteins of Immunological Interest, 5th Ed. Public Health Service, National Institutes of Health, Bethesda, Md. (1991), pages 647-669) and/or those residues from a “hypervariable loop” (for example residues 26-32 (L1), 50-52 (L2), and 91-96 (L3) in the light chain variable domain and 26-32 (H1), 53-55 (H2), and 96-101 (H3) in the heavy chain variable domain; Chothia and Lesk J. Mol. Biol. 196:901-917 (1987)). “Framework” or “FR” residues are those variable domain residues other than the hypervariable region residues as herein defined.

It should be noted that, in the art, more than one system for numbering of amino acid residues is commonly used. The CDRs above are described and numbered according to the Kabat numbering scheme (Kabat, et al., above) but other schemes or sequential numbering may be used. In some cases, sequential and Kabat numbering may be identical.

The hypervariable regions in each chain are held together in close proximity by the FRs and, with the hypervariable regions from the other chain, contribute to the formation of the antigen-binding site of antibodies (see Kabat, et al., above). The constant domains are not involved directly in binding an antibody to an antigen, but exhibit various effector functions, such as participation of the antibody in antibody-dependent cellular toxicity.

A “vector” or “plasmid” or “expression vector” refers to a nucleic acid that can be maintained transiently or stably in a cell to effect expression of one or more recombinant genes. A vector can comprise nucleic acid, alone or complexed with other compounds. A vector optionally comprises viral or bacterial nucleic acids and/or proteins, and/or membranes. Vectors include, but are not limited, to replicons (e.g., RNA replicons, bacteriophages) to which fragments of DNA may be attached and become replicated. Thus, vectors include, but are not limited to, RNA, autonomous self-replicating circular or linear DNA or RNA and include both the expression and non-expression plasmids. Plasmids can be commercially available, publicly available on an unrestricted basis, or can be constructed from available plasmids as reported with published protocols. In addition, the expression vectors may also contain a gene to provide a phenotypic trait for selection of transformed host cells such as dihydrofolate reductase or neomycin resistance for eukaryotic cell culture, or such as tetracycline or ampicillin resistance in E. coli.

Monoclonal antibodies (mAbs) have been shown to be safe and efficacious therapeutic agents. Dozens of therapeutic monoclonal antibodies have been approved for clinical use by the FDA, and additional monoclonal antibodies are in various phases of clinical development for a variety of diseases with the majority targeting various forms of cancer. In general, monoclonal antibodies are generated in non-human mammals. The therapeutic utility of murine monoclonal antibodies is limited, however, principally due to the fact that human patients mount their own antibody response to murine antibodies. This response, the so-called HAMA (human anti-mouse antibody) response, results in the eventual neutralization and rapid elimination of murine monoclonal antibodies. This limitation has been overcome with the development of a process called “humanization” of murine antibodies. Humanization greatly lessens the development of an immune response against the administered therapeutic monoclonal antibodies and thereby avoids the reduction of half-life and therapeutic efficacy consequent on HAMA. For the most part, the humanization process involves grafting the murine complementary determining regions (CDRs) into the framework regions (FRs) of a human immunoglobulin. This strategy is referred to as “CDR grafting.” “Backmutation” to murine amino acid residues of selected residues in the FR is often required to regain affinity that is lost in the initial grafted construct. Fully human antibodies may also be prepared from recombinant mice having human immunoglobulin genes.

Human or humanized antibodies typically have a heavy chain variable domain comprising an amino acid sequence represented by the formula: FR1-CDRH1-FR2-CDRH2-FR3-CDRH3-FR4, wherein “FR1-4” represents the four framework regions and “CDRH1-3” represents the three hypervariable regions of an anti-cysLT antibody variable heavy domain. FR1-4 may be derived from a “consensus sequence” (for example, the most common amino acids of a class, subclass or subgroup of heavy or light chains of human immunoglobulins) as in the examples below or may be derived from an individual human antibody framework region or from a combination of different framework region sequences. Many human antibody framework region sequences are compiled in Kabat, et al., supra, for example. In one embodiment, the variable heavy FR is provided by a consensus sequence of a human immunoglobulin subgroup as compiled by Kabat, et al., supra.

The human variable heavy FR sequence may have substitutions therein, e.g., wherein the human FR residue is replaced by a corresponding nonhuman residue (by “corresponding nonhuman residue” is meant the nonhuman residue with the same Kabat positional numbering as the human residue of interest when the human and nonhuman sequences are aligned), but replacement with the nonhuman residue is not necessary. For example, a replacement FR residue other than the corresponding nonhuman residue may be selected by phage display.

Antibodies typically also have a light chain variable domain comprising an amino acid sequence represented by the formula: FR1-CDRL1-FR2-CDRL2-FR3-CDRL3-FR4, wherein “FR1-4” represents the four framework regions and “CDRL1-3” represents the three hypervariable regions of an anti-cysLT antibody variable light domain. FR1-4 may be derived from a “consensus sequence” (for example the most common amino acids of a class, subclass or subgroup of heavy or light chains of human immunoglobulins) as in the examples below or may be derived from an individual human antibody framework region or from a combination of different framework region sequences. In one preferred embodiment, the variable light FR is provided by a consensus sequence of a human immunoglobulin subgroup as compiled by Kabat, et al., supra.

The human variable light FR sequence may have substitutions therein, e.g., wherein the human FR residue is replaced by a corresponding mouse residue, but replacement with the nonhuman residue is not necessary. For example, a replacement residue other than the corresponding nonhuman residue may be selected by phage display.

The manufacture of monoclonal antibodies is a complex process that stems from the variability of the protein itself. The variability of monoclonal antibodies can be localized to the protein backbone and/or to the carbohydrate moiety. Engineering is commonly applied to antibody molecules to improve their properties, such as enhanced stability, resistance to proteases, aggregation behavior, and to enhance the expression level in heterologous systems.

Agents such as antibodies that reduce the effective concentration of one or more cysLTs are believed to be useful for reducing inflammation, and for treating diseases and conditions, including allergic, cardiovascular, and neurological conditions as well as asthma, cancer, inflammatory diseases and conditions, and diseases and conditions associated with an undesired, excessive, or aberrant level of one or more cysLTs.

In preferred embodiments, the antibodies are monoclonal antibodies. In some of these embodiments, the antibody reduces the effective concentration of one or more of LTE4, LTC4, and/or LTD4. In yet other embodiments, the antibody reduces the effective concentration of one or more of LTE4, LTC4, and LTD4. The effective concentration of one or more of these three cysLTs may be reduced to different extents, or may be reduced substantially equally. Which of these embodiments is preferred may depend on the disease state to be treated.

The therapeutic methods and compositions of the invention are intended to change the relative, absolute, or available concentration(s) of one or more cysLTs. One way to control the amount of undesirable cysLT in a patient is by providing a composition that comprises one or more cysLT binding agents, such as anti-cysLT antibodies, antibody fragments or aptamers, to act as therapeutic “sponges” that reduce the level of free cysLT. This reduction of the effective concentration of cysLT is also referred to as “neutralizing” cysLT. When a compound is stated to be “free,” the compound is not in any way restricted from reaching the site or sites where it exerts its undesirable effects. Typically, a free compound is present in blood and tissue, which either is or contains the site(s) of action of the free compound, or from which a compound can freely migrate to its site(s) of action. A free compound may also be available to be acted upon by any enzyme that converts the compound into an undesirable compound.

Antibodies to cys-LT

The present invention provides compositions and methods relating to anti-cysLT monoclonal antibodies and antigen-binding fragments of such antibodies. Antibodies are typically described as being polyclonal or monoclonal. The anti-cysLT antibodies (or antigen-binding fragments thereof) may be formulated in a pharmaceutical composition that is useful for a variety of purposes, including the treatment of diseases, disorders or physical trauma. Pharmaceutical compositions comprising one or more anti-cysLT antibodies may be incorporated into kits and medical devices for such treatment. Medical devices may be used to administer the pharmaceutical compositions of the invention to a patient in need thereof, and according to some embodiments, kits are provided that include such devices. Such devices and kits may be designed for routine administration, including self-administration, of the pharmaceutical compositions of the invention. Such devices and kits may also be designed for emergency use, for example, in ambulances or emergency rooms, or during surgery, or in activities where injury or illness, e.g., asthma “attack,” is possible but where full medical attention may not be immediately forthcoming (for example, hiking and camping, or sports or combat situations).

Anti-cysLT antibodies (and antigen-binding fragments thereof) are also useful for diagnostics and as research reagents, and may be formulated and/or packaged accordingly. The anti-cysLT antibody may be attached to a solid support for research or diagnostic use. Columns, beads, and ELISA plates are examples of solid supports.

### Antibody Generation and Characterization

Monoclonal antibodies to cysLT may be made as described in the examples below. In one embodiment, the monoclonal antibodies to cysLT are those with strong binding affinity for one or more of the cysLTs. Antibody affinities may be determined as described in the examples hereinbelow. It may be desirable to select an antibody with preferential or specific affinity for one cysLT, e.g., LTC4, LTD4, or LTE4. In other embodiments it may be desirable to select an antibody with affinity for more than one of the aforementioned cysLTs, or for all three. The antibody may bind multiple cysLTs with differing affinities. It may also be desirable to select chimeric or humanized antibodies or antigen-binding antibody fragments which have other beneficial properties from a therapeutic perspective. For example, the antibody may be one that reduces an inflammatory response or angiogenesis.

Preferably the humanized antibody or fragment thereof fails to elicit an immunogenic response upon administration of a therapeutically effective amount of the antibody to a human patient. If an immunogenic response is elicited, preferably the response will be such that the antibody still provides a therapeutic benefit to the patient treated therewith.

A. Antibody Preparation

Methods for generating anti-cysLT antibodies, including monoclonal antibodies, are described in the Examples below. Exemplary techniques for generating such nonhuman antibody and parent antibodies will be described in the following sections.

(i) Antigen Preparation.

The antigen to be used for production of antibodies may be, e.g., an intact cysLT or a portion of a cysLT, e.g., a cysLT fragment comprising the desired native epitope. In one embodiment, the antigen is a cysLT which is conjugated to a carrier, forming an antibody conjugate. Other forms of cysLT antigens useful for generating antibodies will be apparent to those skilled in the art.

(ii) Polyclonal Antibodies.

Polyclonal antibodies are typically raised in animals by multiple injections, such as subcutaneous (sc) or intraperitoneal (ip) injections, of the relevant antigen and an adjuvant. Typically, using this method, the lipid antigen is linked to a carrier such as a protein that is immunogenic in the species to be immunized, e.g., keyhole limpet hemocyanin (KLH), ovalbumin (OVA), serum albumin, bovine thyroglobulin, or soybean trypsin inhibitor, using a bifunctional or derivatizing agent (also referred to as a linker), for example, maleimidobenzoyl sulfosuccinimide ester (conjugation through cysteine residues), N-hydroxysuccinimide (through lysine residues), glutaraldehyde, succinic anhydride, SOCl₂, or R¹N═C═NR, where R and R¹ are different alkyl groups. Other linkers known in the art include the following heterobifunctional crosslinkers (Thermo Scientific, Waltham Mass.) that reacts with primary amines and sulfhydryl groups: succinimidyl 4-[N-maleimidomethyl]cyclohexane-1-carboxylate (SMCC), succinimidyl iodoacetate (SIA), and succinimidyl (4-iodoacetyl)aminobenzoate (SIAB). The native cysLT may also be directly conjugated to a carrier protein using the crosslinking agent 1-ethyl-3-[3-dimethylaminopropyl]carbodiimide hydrochloride (EDC, Thermo Scientific, Waltham Mass.) and used, e.g., in screening assays for anti-cysLT antibody. Non-protein carriers (e.g., colloidal gold, polyethylene glycol, silicone beads) are also known in the art for use in antibody production.

In one typical protocol, animals (e.g., mice or rabbits) are immunized against the cysLT immunogen (e.g., antigen, immunogenic conjugates, or derivatives) by combining, e.g., 100 ug or 5 ug of the protein or conjugate (for rabbits or mice, respectively) with three volumes of Freund's complete adjuvant and injecting the solution intradermally at multiple sites. Typically, about one month later the animals are boosted with ⅕ to 1/10 the original amount of peptide or conjugate in Freund's complete adjuvant by subcutaneous injection at multiple sites. Seven to 14 days later the animals are bled and the serum is assayed for antibody titer. Animals are boosted until the titer plateaus. Preferably, the animal is boosted with the conjugate of the same antigen, but conjugated to a different protein and/or through a different cross-linking reagent. Aggregating agents such as alum may be suitably used to enhance the immune response. Conjugates also can be made in recombinant cell culture as protein fusions.

(iii) Monoclonal Antibodies.

Methods for making monoclonal antibodies are known in the art. For example, monoclonal antibodies may be made using the hybridoma method first described by Kohler, et al. (1975), Nature, 256:495, or may be made by recombinant DNA methods (U.S. Pat. No. 4,816,567. In the hybridoma method, a mouse, rabbit or other appropriate host animal, such as a hamster or macaque monkey, is immunized as hereinabove described to elicit lymphocytes that produce or are capable of producing antibodies that will specifically bind to the protein used for immunization. Alternatively, lymphocytes may be immunized in vitro. Lymphocytes then are fused with myeloma cells using a suitable fusing agent, such as polyethylene glycol, to form a hybridoma cell (coding, Monoclonal Antibodies: Principles and Practice, pp. 59-103 (Academic Press, 1986)).

The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Preferred myeloma cells are those that fuse efficiently, support stable high-level production of antibody by the selected antibody-producing cells, and are sensitive to a medium such as HAT medium. Among these, preferred myeloma cell lines are murine myeloma lines, such as those derived from MOP-21 and M.C.-11 mouse tumors available from the Salk Institute Cell Distribution Center, San Diego, Calif. USA, and SP-2 or X63-Ag8-653 cells available from the American Type Culture Collection, Rockville, Md. USA. Human myeloma and mouse-human heteromyeloma cell lines also have been described for the production of human monoclonal antibodies (Kozbor, J. Immunol., 133:3001 (1984); Brodeur, et al., Monoclonal Antibody Production Techniques and Applications, pp. 51-63 (Marcel Dekker, Inc., New York, 1987)).

Culture medium in which hybridoma cells are growing is assayed for production of monoclonal antibodies directed against the antigen. Preferably, the binding specificity of monoclonal antibodies produced by hybridoma cells is determined by immunoprecipitation or by an in vitro binding assay, such as radioimmunoassay (RIA) or enzyme-linked immunoabsorbent assay (ELISA). The binding affinity of the monoclonal antibody can, for example, be determined by the Scatchard analysis of Munson, et al., Anal. Biochem., 107:220 (1980).

After hybridoma cells are identified that produce antibodies of the desired specificity, affinity, and/or activity, the clones may be subcloned by limiting dilution procedures and grown by standard methods (coding, Monoclonal Antibodies: Principles and Practice, pp. 59-103 (Academic Press, 1986)). Suitable culture media for this purpose include, for example, D-MEM or RPMI-1640 medium. In addition, the hybridoma cells may be grown in vivo as ascites tumors in an animal.

The monoclonal antibodies secreted by the subclones are suitably separated from the culture medium, ascites fluid, or serum by conventional immunoglobulin purification procedures such as, for example, protein A-Sepharose, hydroxylapatite chromatography, gel electrophoresis, dialysis, or affinity chromatography.

DNA encoding the monoclonal antibodies is readily isolated and sequenced using conventional procedures (e.g., by using oligonucleotide probes that are capable of binding specifically to genes encoding the heavy and light chains of the monoclonal antibodies). The hybridoma cells serve as a preferred source of such DNA. Once isolated, the DNA may be placed into expression vectors, which are then transfected into host cells such as E coli cells, simian COS cells, Chinese hamster ovary (CHO) cells, or myeloma cells that do not otherwise produce immunoglobulin protein, to obtain the synthesis of monoclonal antibodies in the recombinant host cells. Recombinant production of antibodies will be described in more detail below.

(iv) Humanization and Amino Acid Sequence Variation.

Some preferred embodiments of the invention utilize humanized antibodies to one or more cysLTs. General methods for humanization of antibodies are described in, e.g., U.S. Pat. No. 5,861,155, U.S. Pat. No. 6,479,284, U.S. Pat. No. 6,407,213, U.S. Pat. No. 6,639,055, U.S. Pat. No. 6,500,931, U.S. Pat. No. 5,530,101, U.S. Pat. No. 5,585,089, U.S. Pat. No. 5,693,761, U.S. Pat. No. 5,693,762, U.S. Pat. No. 6,180,370, U.S. Pat. No. 5,714,350, U.S. Pat. No. 6,350,861, U.S. Pat. No. 5,777,085, U.S. Pat. No. 5,834,597, U.S. Pat. No. 5,882,644, U.S. Pat. No. 5,932,448, U.S. Pat. No. 6,013,256, U.S. Pat. No. 6,129,914, U.S. Pat. No. 6,210,671, U.S. Pat. No. 6,329,511, US2003166871, U.S. Pat. No. 5,225,539, U.S. Pat. No. 6,548,640 and U.S. Pat. No. 5,624,821. In certain embodiments, it may be desirable to generate antibodies with amino acid sequence variations compared to that of the initially obtained (parent) humanized antibodies, particularly where these improve the binding affinity or other biological properties of the antibody. This may be referred to as “optimization” of the parent antibody.

Antibodies with amino acid sequence variations may be prepared by introducing appropriate nucleotide changes (mutations) into the antibody DNA, or by peptide synthesis. Such variations include, for example, deletions from, and/or insertions into and/or substitutions of, residues within the amino acid sequences. Any combination of deletion, insertion, and substitution is made to arrive at the final construct, provided that the final construct possesses the desired characteristics. The amino acid changes also may alter post-translational processes of the antibody, such as changing the number or position of glycosylation sites. Other types of post-translational processing of proteins (including antibodies) include deamidation, a nonenzymatic process, and C-terminal lysine or arginine clipping, which are enzymatic processes and are fairly common in monoclonal antibodies and other recombinant proteins isolated from mammalian cells. Harris R J. (1995) J Chromatogr A 705:129-134. Amino acid sequences herein are provided irrespective of any possible post-translational modifications that may or may not occur under given conditions.

A useful method for identification of certain residues or regions of the antibody that are preferred locations for mutagenesis is called “alanine scanning mutagenesis,” as described by Cunningham and Wells (1989), Science, 244:1081-1085. Here, a residue or group of target residues are identified (e.g., charged residues such as arg, asp, his, lys, and glu) and replaced by a neutral or negatively charged amino acid (most preferably alanine or polyalanine) to affect the interaction of the antibody with the antigen. Those amino acid locations in the antibody that demonstrate functional sensitivity to the substitutions then are refined by introducing further or other substitutions or modifications at, or for, the sites of substitution. Thus, while the site for introducing an amino acid sequence variation is predetermined, the nature of the mutation per se need not be predetermined. For example, to analyze the performance of a mutation at a given site, ala scanning or random mutagenesis is conducted at the target codon or region and the resulting antibodies are expressed and screened for the desired activity. Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an N-terminal methionyl residue or the antibody fused to an epitope tag. Other insertions include the fusion of an enzyme or a polypeptide which increases the serum half-life of the antibody to the N- or C-terminus of the antibody.

Another type of antibody mutation is an amino acid substitution. These mutated antibodies have at least one amino acid residue removed from the antibody molecule and a different residue inserted in its place. The sites of greatest interest for substitutional mutagenesis include the hypervariable regions, but framework alterations are also contemplated. Conservative substitutions are preferred, but if such substitutions result in a change in biological activity, then more substantial changes may be introduced and the products screened. Such substitutions and their degree of conservativeness are well known in the art. Substantial modifications in the biological properties of the antibody are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are divided into groups based on common side-chain properties:

(1) hydrophobic: norleucine, met, ala, val, leu, ile;

(2) neutral hydrophilic: cys, ser, thr;

(3) acidic: asp, glu;

(4) basic: asn, gln, his, lys, arg;

(5) residues that influence chain orientation: gly, pro; and

(6) aromatic: trp, tyr, phe.

Non-conservative substitutions entail exchanging a member of one of these classes for another class.

Any cysteine residue not involved in maintaining the proper conformation of the antibody also may be substituted, to improve the oxidative stability of the molecule and prevent aberrant crosslinking. Conversely, cysteine bond(s) may be added to the antibody to improve its stability (particularly where the antibody is an antibody fragment such as an Fv fragment).

One type of substitution involves substituting one or more hypervariable region residues of a parent antibody (e.g., a humanized or human antibody). Generally, the resulting antibody(ies) selected for further development will have improved biological properties relative to the parent antibody from which they are generated, and is often referred to as an “optimized” antibody. A convenient way for generating antibodies with such substitutions is affinity maturation using phage display. Briefly, several hypervariable region sites (e.g., 6-7 sites) are mutated to generate all possible amino substitutions at each site. The antibodies thus generated are displayed in a monovalent fashion from filamentous phage particles as fusions to the gene IIII product of M13 packaged within each particle. The phage-displayed antibodies are screened for their biological activity (e.g., binding affinity). In order to identify candidate hypervariable region sites for modification, alanine scanning mutagenesis can be performed to identify hypervariable region residues contributing significantly to antigen binding. Alternatively, or in addition, it may be beneficial to analyze a crystal structure of the antigen-antibody complex to identify contact points between the antibody and antigen. Such contact residues and neighboring residues are candidates for substitution according to the techniques elaborated herein. Once antibodies with such substitutions are generated, they may be subjected to screening as described herein and antibodies with superior properties in one or more relevant assays may be selected for further development.

Another type of amino acid change of the antibody alters the original glycosylation pattern of the antibody. By “altering” is meant deleting one or more carbohydrate moieties found in the antibody, and/or adding one or more glycosylation sites that are not present in the antibody.

Glycosylation of antibodies is typically either N-linked and/or or O-linked. N-linked refers to the attachment of the carbohydrate moiety to the side chain of an asparagine residue. The tripeptide sequences asparagine-X-serine and asparagine-X-threonine, where X is any amino acid except proline, are the most common recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-aceylgalactosamine, galactose, or xylose to a hydroxyamino acid, most commonly serine or threonine, although 5-hydroxyproline or 5-hydroxylysine may also be used.

Addition of glycosylation sites to the antibody is conveniently accomplished by altering the amino acid sequence such that it contains one or more of the above-described tripeptide sequences (for N-linked glycosylation sites). The alteration may also be made by the addition of, or substitution by, one or more serine or threonine residues to the sequence of the original antibody (for O-linked glycosylation sites).

Nucleic acid molecules encoding antibody amino acid sequences are prepared by a variety of methods known in the art. These methods include, but are not limited to, isolation from a natural source or preparation by oligonucleotide-mediated (or site-directed) mutagenesis, PCR mutagenesis, and cassette mutagenesis of an earlier prepared antibody.

(v) Human Antibodies.

As an alternative to humanization, human antibodies can be generated. For example, it is now possible to produce transgenic animals (e.g., mice) that are capable, upon immunization, of producing a full repertoire of human antibodies in the absence of endogenous immunoglobulin production. For example, it has been described that the homozygous deletion of the antibody heavy-chain joining region (J_{H}) gene in chimeric and germ-line mutant mice results in complete inhibition of endogenous antibody production. Transfer of the human germ-line immunoglobulin gene array in such germ-line mutant mice will result in the production of human antibodies upon antigen challenge. See, e.g., Jakobovits, et al. (1993), Proc. Natl. Acad. Sci. USA, 90:2551; Jakobovits, et al. 1993), Nature, 362:255-258; Bruggermann, et al. (1993), Year in Immuno., 7:33; and U.S. Pat. Nos. 5,591,669, 5,589,369, and 5,545,807. Human antibodies can also be derived from phage-display libraries (Hoogenboom, et al. (1991), J. Mol. Biol., 227:381; Marks, et al. (1991), J. Mol. Biol., 222:581-597; and U.S. Pat. Nos. 5,565,332 and 5,573,905). As discussed above, human antibodies may also be generated by in vitro activated B cells (see U.S. Pat. Nos. 5,567,610 and 5,229,275).

(vi) Antibody Fragments.

In certain embodiments, the anti-cysLT agent is an antigen-binding antibody fragment. Various techniques have been developed for the production of antigen-binding antibody fragments. Traditionally, these fragments were derived via proteolytic digestion of intact antibodies (see, e.g., Morimoto, et al. (1992), Journal of Biochemical and Biophysical Methods 24:107-117, and Brennan, et al. (1985), Science 229:81). However, these fragments can now be produced directly by recombinant host cells. For example, Fab′-SH fragments can be directly recovered from E. coli and chemically coupled to form F(ab′)₂ fragments (Carter, et al. (1992), Bio/Technology 10:163-167). In another embodiment, the F(ab′)₂ is formed using the leucine zipper GCN4 to promote assembly of the F(ab′)₂ molecule. According to another approach, Fv, Fab or F(ab′)₂ fragments can be isolated directly from recombinant host cell culture. Other techniques for the production of antibody fragments will be apparent to the skilled practitioner.

B. Vectors, Host Cells and Recombinant Methods

For recombinant production of an antibody, the nucleic acid(s) encoding it may be isolated and inserted into a replicable vector for further cloning (amplification of the DNA) or for expression. In another embodiment, the antibody may be produced by homologous recombination, e.g., as described in U.S. Pat. No. 5,204,244, which is specifically incorporated herein by reference. DNA encoding the monoclonal antibody is readily isolated and sequenced using conventional procedures (e.g., by using oligonucleotide probes that are capable of binding specifically to genes encoding the heavy and light chains of the antibody). Many vectors are available. The vector components generally include, but are not limited to, one or more of the following: a signal sequence, an origin of replication, one or more marker genes, an enhancer element, a promoter, and a transcription termination sequence, e.g., as described in U.S. Pat. No. 5,534,615, which is specifically incorporated herein by reference.

Suitable host cells for cloning or expressing the DNA in the vectors herein are the prokaryote, yeast, or higher eukaryote cells described above. Suitable prokaryotes for this purpose include eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis (e.g., B. licheniformis 41P disclosed in DD 266,710 published 12 Apr. 1989), Pseudomonas such as P. aeruginosa, and Streptomyces. One preferred E. coli cloning host is E. coli 294 (ATCC 31,446), although other strains such as E. coli B, E. coli X1776 (ATCC 31,537), and E. coli W3110 (ATCC 27,325) are suitable. These examples are illustrative rather than limiting.

In addition to prokaryotes, eukaryotic microbes such as filamentous fungi or yeast are suitable cloning or expression hosts for antibody-encoding vectors. Saccharomyces cerevisiae, or common baker's yeast, is the most commonly used among lower eukaryotic host microorganisms. However, a number of other genera, species, and strains are commonly available and useful herein, such as Schizosaccharomyces pombe; Kluyveromyces hosts such as, e.g., K. lactis, K. fragilis (ATCC 12,424), K. bulgaricus (ATCC 16,045), K. wickeramii (ATCC 24,178), K. waltii (ATCC 56,500), K. drosophilarum (ATCC 36,906), K. thermotolerans, and K. marxianus; yarrowia (EP 402,226); Pichia pastoris (EP 183,070); Candida; Trichoderma reesia (EP 244,234); Neurospora crassa; Schwanniomyces such as Schwanniomyces occidentalis; and filamentous fungi such as, e.g., Neurospora, Penicillium, Tolypocladium, and Aspergillus hosts such as A. nidulans and A. niger.

Suitable host cells for the expression of glycosylated antibodies are derived from multicellularorganisms. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruitfly), and Bombyx mori have been identified. A variety of viral strains for transfection are publicly available, e.g., the L-1 variant of Autographa californica NPV and the Bm-5 strain of Bombyx mori NPV, and such viruses may be used, particularly for transfection of Spodoptera frugiperda cells. Plant cell cultures of cotton, corn, potato, soybean, petunia, tomato, and tobacco can also be utilized as hosts.

However, interest has been greatest in vertebrate cells, and propagation of vertebrate cells in culture (tissue culture) has become a routine procedure. Examples of useful mammalian host cell lines are monkey kidney CV1 line transformed by SV40 (COS-7, ATCC CRL 1651); human embryonic kidney line (293 or 293 cells subcloned for growth in suspension culture, Graham, et al. (1977), J. Gen Virol. 36:59); baby hamster kidney cells (BHK, ATCC CCL 10); Chinese hamster ovary cells/−DHFR (CHO, Urlaub, et al. (1980), Proc. Natl. Acad. Sci. USA 77:4216); mouse Sertoli cells (TM4, Mather (1980), Biol. Reprod. 23:243-251); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather, et al. (1982), Annals N.Y. Acad. Sci. 383:44-68); MRC 5 cells; FS4 cells; and a human hepatoma line (Hep G2).

Host cells are transformed with the above-described expression or cloning vectors for antibody production and cultured in conventional nutrient media modified as appropriate for inducing promoters, selecting transformants, or amplifying the genes encoding the desired sequences.

The host cells used to produce antibody may be cultured in a variety of media. Commercially available media such as Ham's F10 (Sigma), Minimal Essential Medium ((MEM), (Sigma), RPMI-1640 (Sigma), and Dulbecco's Modified Eagle's Medium ((DMEM), Sigma) are suitable for culturing the host cells. In addition, any of the media described in Ham, et al. (1979), Meth. Enz. 58:44, Barnes, et al. (1980), Anal. Biochem. 102:255, U.S. Pat. Nos. 4,767,704; 4,657,866; 4,927,762; 4,560,655; or 5,122,469; WO 90/03430; WO 87/00195; or U.S. Pat. reexam. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

When using recombinant techniques, the antibody can be produced intracellularly, in the periplasmic space, or directly secreted into the medium. If the antibody is produced intracellularly, as a first step, the particulate debris, either host cells or lysed fragments, is removed, for example, by centrifugation or ultrafiltration. Carter, et al. (1992) (Bio/Technology 10:163-167) describe a procedure for isolating antibodies that are secreted to the periplasmic space of E. coli. Briefly, cell paste is thawed in the presence of sodium acetate (pH 3.5), EDTA, and phenylmethylsulfonylfluoride (PMSF) over about 30 min. Cell debris can be removed by centrifugation. Where the antibody is secreted into the medium, supernatants from such expression systems are generally first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

The antibody composition prepared from the cells can be purified using, for example, hydroxylapatite chromatography, gel electrophoresis, dialysis, and affinity chromatography, with affinity chromatography being the preferred purification technique. The suitability of protein A as an affinity ligand depends on the species and isotype of any immunoglobulin Fc domain that is present in the antibody. Protein A can be used to purify antibodies that are based on human heavy chains (Lindmark, et al. (1983), J. Immunol. Meth. 62:1-13). Protein G is recommended for all mouse isotypes and for human γ3 (Guss, et al. (1986), EMBO J. 5:15671575). The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the antibody comprises a C_{H3} domain, the Bakerbond ABX™ resin (J. T. Baker, Phillipsburg, N.J.) is useful for purification. Other techniques for protein purification, such as fractionation on an ion-exchange column, ethanol precipitation, Reverse Phase HPLC, chromatography on silica, chromatography on heparin SEPHAROSE™, chromatography on an anion or cation exchange resin (such as a polyaspartic acid column), chromatofocusing, SDS-PAGE, and ammonium sulfate precipitation are also available depending on the antibody to be recovered.

Following any preliminary purification step(s), the mixture comprising the antibody of interest and contaminants may be subjected to low pH hydrophobic interaction chromatography using an elution buffer at a pH between about 2.5-4.5, preferably performed at low salt concentrations (e.g., from about 0-0.25M salt).

C. Pharmaceutical Formulations

Therapeutic formulations of the antibody are prepared for storage by mixing the antibody having the desired degree of purity with optional physiologically acceptable carriers, excipients or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

The formulation herein may also contain more than one active compound as necessary for the particular indication being treated, preferably those with complementary activities that do not adversely affect each other. Such molecules are suitably present in combination in amounts that are effective for the purpose intended.

The active ingredients may also be entrapped in microcapsule prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

The formulations to be used for in vivo administration must be sterile. This is readily accomplished for instance by filtration through sterile filtration membranes.

Sustained-release preparations may be prepared. Suitable examples of sustained-release preparations include semipermeable matrices of solid hydrophobic polymers containing the antibody, which matrices are in the form of shaped articles, e.g., films, or microcapsule. Examples of sustained-release matrices include polyesters, hydrogels (for example, poly(2-hydroxyethyl-methacrylate), or poly(vinyl alcohol)), polylactides (U.S. Pat. No. 3,773,919), copolymers of L-glutamic acid and .gamma. ethyl-L-glutamate, non-degradable ethylene-vinyl acetate, degradable lactic acid-glycolic acid copolymers such as the Lupron Depot™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated antibodies remain in the body for a long time, they may denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

Various excipients might also be added to the formulated antibody to improve performance of the therapy, make the therapy more convenient or to clearly ensure that the formulated antibody is used only for its intended, approved purpose. Examples of excipients include chemicals to control pH, antimicrobial agents, preservatives to prevent loss of antibody potency, dyes to identify the formulation for airway use only, solubilizing agents to increase the concentration of antibody in the formulation, penetration enhancers and the use of agents to adjust isotonicity and/or viscosity. Inhibitors of, e.g., proteases, can be added to prolong the half life of the antibody, if desired.

D. Non-Therapeutic Uses for the Antibodies

The antibodies disclosed herein may be used as affinity purification agents. In this process, the antibodies are immobilized on a solid phase such a Sephadex resin or filter paper, using methods well known in the art. The immobilized antibody is contacted with a sample containing the cysLT to be purified, and thereafter the support is washed with a suitable solvent that will remove substantially all the material in the sample except the cys-LT, which is bound to the immobilized antibody. Finally, the support is washed with another suitable solvent, such as glycine buffer, for instance between pH 3 to pH 5.0, that will release the cysLT from the antibody.

Anti-cysLT antibodies may also be useful in diagnostic assays for cysLT(s), e.g., detecting its expression in specific cells, tissues, or bodily fluids. Such diagnostic methods may be useful in diagnosis, particularly early diagnosis, e.g., of an airway disease or disorder.

For diagnostic applications, the antibody may be labeled with a detectable moiety. Numerous labels are available which can be generally grouped into the following categories:

(a) Radioisotopes, such as ³⁵S, ¹⁴C, ¹²⁵I, ³H, and ¹³¹I. The antibody can be labeled with the radioisotope using the techniques described in Current Protocols in Immunology, Volumes 1 and 2, Coligen et al., Ed. Wiley-Interscience, New York, N.Y., Pubs. (1991), for example, and radioactivity can be measured using scintillation counting.

(b) Fluorescent labels such as rare earth chelates (europium chelates) or fluorescein and its derivatives, rhodamine and its derivatives, dansyl, Lissamine, phycoerythrin and Texas Red are available. The fluorescent labels can be conjugated to the antibody using the techniques disclosed in Current Protocols in Immunology, supra, for example. Fluorescence can be quantified using a fluorimeter.

(c) Various enzyme-substrate labels are available and U.S. Pat. No. 4,275,149 provides a review of some of these. The enzyme generally catalyzes a chemical alteration of the chromogenic substrate that can be measured using various techniques. For example, the enzyme may catalyze a color change in a substrate, which can be measured spectrophotometrically. Alternatively, the enzyme may alter the fluorescence or chemiluminescence of the substrate. Techniques for quantifying a change in fluorescence are described above. The chemiluminescent substrate becomes electronically excited by a chemical reaction and may then emit light that can be measured (using a chemiluminometer, for example) or donates energy to a fluorescent acceptor. Examples of enzymatic labels include luciferases (e.g., firefly luciferase and bacterial luciferase; U.S. Pat. No. 4,737,456), luciferin, 2,3-dihydrophthalazinediones, malate dehydrogenase, urease, peroxidase such as horseradish peroxidase (HRPO), alkaline phosphatase, beta-galactosidase, glucoamylase, lysozyme, saccharide oxidases (e.g., glucose oxidase, galactose oxidase, and glucose-6-phosphate dehydrogenase), heterocyclicoxidases (such as uricase and xanthine oxidase), lactoperoxidase, microperoxidase, and the like. Techniques for conjugating enzymes to antibodies are described in O'Sullivan, et al. (1981), Methods for the Preparation of Enzyme-Antibody Conjugates for use in Enzyme Immunoassay, in Methods in Enzym. (ed J. Langone & H. Van Vunakis), Academic press, New York, 73:147-166.

Examples of enzyme-substrate combinations include, for example:

(i) Horseradish peroxidase (HRPO) with hydrogen peroxidase as a substrate, wherein the hydrogen peroxidase oxidizes a dye precursor (e.g., orthophenylene diamine (OPD) or 3,3′,5,5′-tetramethyl benzidine hydrochloride (TMB));

(ii) alkaline phosphatase (AP) with para-Nitrophenyl phosphate as chromogenic substrate; and (iii) .beta.-D-galactosidase (.beta.-D-Gal) with a chromogenic substrate (e.g., p-nitrophenyl-β-D-galactosidase) or fluorogenic substrate 4-methylumbelliferyl-.beta.-D-galactosidase.

Numerous other enzyme-substrate combinations are available to those skilled in the art. For a general review of these, see U.S. Pat. Nos. 4,275,149 and 4,318,980.

Sometimes, the label is indirectly conjugated with the antibody. The skilled artisan will be aware of various techniques for achieving this. For example, the antibody can be conjugated with biotin and any of the three broad categories of labels mentioned above can be conjugated with avidin, or vice versa. Biotin binds selectively to avidin and thus, the label can be conjugated with the antibody in this indirect manner. Alternatively, to achieve indirect conjugation of the label with the antibody, the antibody is conjugated with a small hapten (e.g., digoxin) and one of the different types of labels mentioned above is conjugated with an anti-hapten antibody (e.g., anti-digoxin antibody). Thus, indirect conjugation of the label with the antibody can be achieved.

In another embodiment, the anti-cysLT antibody need not be labeled, and the presence thereof can be detected, e.g., using a labeled antibody which binds to the anti-cysLT antibody.

The antibodies disclosed herein may be employed in any known assay method, such as competitive binding assays, direct and indirect sandwich assays, and immunoprecipitation assays. Zola (1987), Monoclonal Antibodies: A Manual of Techniques, pp. 147-158 (CRC Press, Inc.

Competitive binding assays rely on the ability of a labeled standard to compete with the test sample analyte for binding with a limited amount of antibody. The amount of cysLT in the test sample is inversely proportional to the amount of standard that becomes bound to the antibodies. To facilitate determining the amount of standard that becomes bound, the antibodies generally are insoluble before or after the competition, so that the standard and analyte that are bound to the antibodies may conveniently be separated from the standard and analyte that remain unbound.

Sandwich assays involve the use of two antibodies, each capable of binding to a different immunogenic portion, or epitope, of the protein to be detected. In a sandwich assay, the test sample analyte is bound by a first antibody that is immobilized on a solid support, and thereafter a second antibody binds to the analyte, thus forming an insoluble three-part complex. See, e.g., U.S. Pat. No. 4,376,110. The second antibody may itself be labeled with a detectable moiety (direct sandwich assays) or may be measured using an anti-immunoglobulin antibody that is labeled with a detectable moiety (indirect sandwich assay). For example, one type of sandwich assay is an ELISA assay, in which case the detectable moiety is an enzyme.

For immunohistochemistry, the blood or tissue sample may be fresh or frozen or may be embedded in paraffin and fixed with a preservative such as formalin, for example.

The antibodies may also be used for in vivo diagnostic assays. Generally, the antibody is labeled with a radionuclide (such as ¹¹¹In, ⁹⁹Tc, ¹⁴C, ¹³¹I, ¹²⁵I, ³H, ³²P, or ³⁵S) so that the bound target molecule can be localized using immunoscintigraphy.

E. Diagnostic Kits

As a matter of convenience, the antibodies disclosed herein can be provided in a kit, such as an ELISA kit; for example, a packaged combination of reagents in predetermined amounts with instructions for performing the diagnostic assay. Where the antibody is labeled with an enzyme, the kit will include substrates and cofactors required by the enzyme (e.g., a substrate precursor which provides the detectable chromophore or fluorophore). In addition, other additives may be included such as stabilizers, buffers (e.g., a block buffer or lysis buffer) and the like. The relative amounts of the various reagents may be varied widely to provide for concentrations in solution of the reagents which substantially optimize the sensitivity of the assay. Particularly, the reagents may be provided as dry powders, usually lyophilized, including excipients which on dissolution will provide a reagent solution having the appropriate concentration.

F. Therapeutic Uses for the Antibody

For therapeutic applications, the anti-cysLT antibodies (and cysLT-binding antibody fragments) described herein are administered to a mammal, preferably a human, in a pharmaceutically acceptable dosage form such as those discussed above, including those that may be administered to a human intravenously as a bolus or by continuous infusion over a period of time, or by intramuscular, intraperitoneal, intra-cerebrospinal, subcutaneous, intra-articular, intrasynovial, intrathecal, intranasal, oral, topical, ocular, periocular, intravitreal, or inhalation routes. For the latter, an antibody can be delivered to the airway, for example, by use of a metered dose inhaler with or without spacer, a dry powder inhaler, a breath-actuated metered dose inhaler, or a nebulizer.

For the prevention or treatment of disease, the appropriate dosage of antibody will depend on the type of disease to be treated, as defined above, the severity and course of the disease, whether the antibody is administered for preventive or therapeutic purposes, previous therapy, the patient's clinical history and response to the antibody, and the discretion of the attending physician. The antibody is suitably administered to the patient at one time or over a series of treatments.

Depending on the type and severity of the disease, about 1 μg/kg to about 50 mg/kg (e.g., 0.1-20 mg/kg) of antibody is an initial candidate dosage for administration to the patient, whether, for example, by one or more separate administrations, or by continuous infusion. A typical daily or weekly dosage might range from about 1 μg/kg to about 20 mg/kg or more, depending on the factors mentioned above. For repeated administrations over several days or longer, depending on the condition, the treatment is repeated until a desired suppression of disease symptoms occurs. However, other dosage regimens may be useful. The progress of this therapy is easily monitored by conventional techniques and assays, including, for example, radiographic imaging. Detection methods using the antibody to determine cysLT levels in bodily fluids or tissues may be used in order to optimize patient exposure to the therapeutic antibody.

According to another embodiment, the effectiveness of the antibody in preventing or treating disease may be improved by administering the antibody serially or in combination with another agent that is effective for those purposes, such as a chemotherapeutic drug for treatment of cancer, or a drug for treatment of ocular disease. Such other agents may be present in the composition being administered or may be administered separately. Also, the antibody is suitably administered serially or in combination with the other agent or modality, e.g., chemotherapeutic drug or radiation for treatment of cancer.

G. Articles of Manufacture

In another embodiment, an article of manufacture containing materials useful for the treatment of the disorders described above is provided. The article of manufacture comprises a container and a label. Suitable containers include, for example, bottles, vials, syringes, and test tubes. The containers may be formed from a variety of materials such as glass or plastic. The container holds a composition that is effective for treating the condition and may have a sterile access port (for example, the container may be an intravenous solution bag, a vial having a stopper pierceable by a hypodermic injection needle, or a dropper bottle). The active agent in the composition is the anti-cysLT antibody. The label on, or associated with, the container indicates that the composition is used for treating the condition of choice. The article of manufacture may further comprise a second container comprising a pharmaceutically-acceptable buffer, such as phosphate-buffered saline, Ringer's solution and dextrose solution. It may further include other materials desirable from a commercial and user standpoint, including other buffers, diluents, filters, needles, syringes, and package inserts with instructions for use. For airway administration, devices such as nebulizers or inhalers are used, and the latter may be designed either for liquid or dry powder drug administration. The article of manufacture may also be a kit such as an ELISA kit utilizing anti-cysLT antibody for detection of cysLT, e.g., in bodily fluids.

Other articles of the invention include kits that contain a pharmaceutical composition of the invention in a suitable container (e.g., a labeled glass vial or ampule), preferably packaged in a container that includes instructions for use of the composition (e.g., a pharmaceutical product package insert).

The invention will be better understood by reference to the following Examples, which are intended to merely illustrate the best mode now known for practicing the invention. The scope of the invention is not to be considered limited thereto.

### EXAMPLES

### Example 1

### Synthesis of Immunogen (LTE4-Protein Complex)

An LTE4-protein complex for use as an immunogen was prepared by crosslinking LTE4 via the amine located in the head group of LTE4 to a protein carrier using bis(sulfosuccinimidyl)-suberate, a homobifunctional amine-to-amine crosslinker. 0.22 mg of cysteinyl leukotriene E4 (LTE4; Cayman Chemical Company, Cat #20410) was incubated with 2.5 mg of Imject Blue Carrier Protein (BCP; Thermo Scientific, Cat #77130) and 2.9 mg of bis(sulfosuccinimidyl)suberate (BS3; Thermo Scientific, Cat #21580) in 90% PBS/10% DMSO for 2 hours at room temperature, followed by purification of the protein-lipid conjugate using a desalting column (Thermo Scientific, part #89882) equilibrated with Imject purification buffer (Thermo Scientific, part #77159).

### Example 2

### Antibody Production

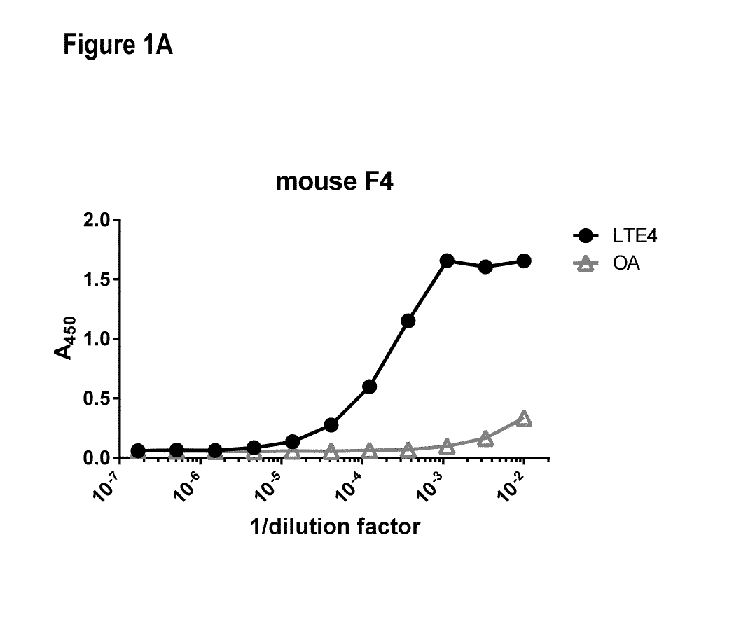

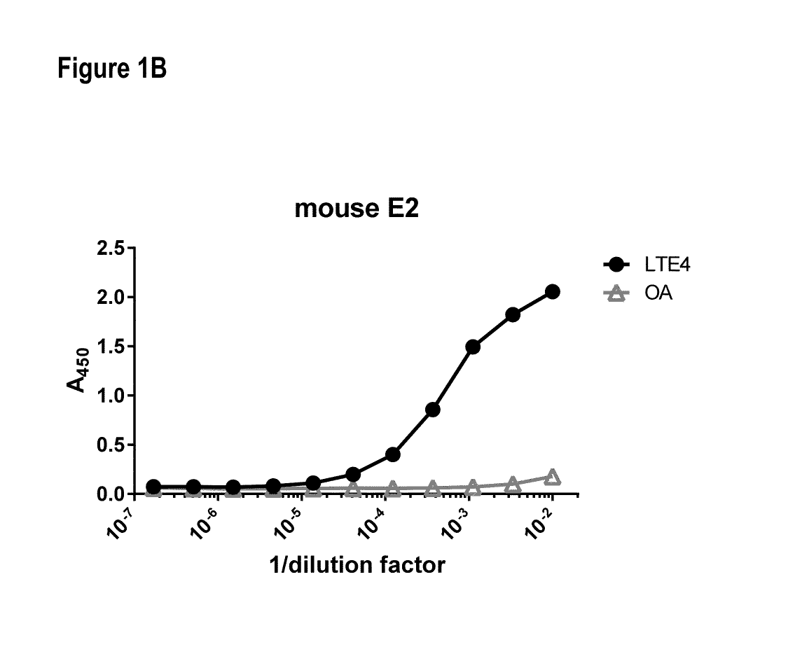

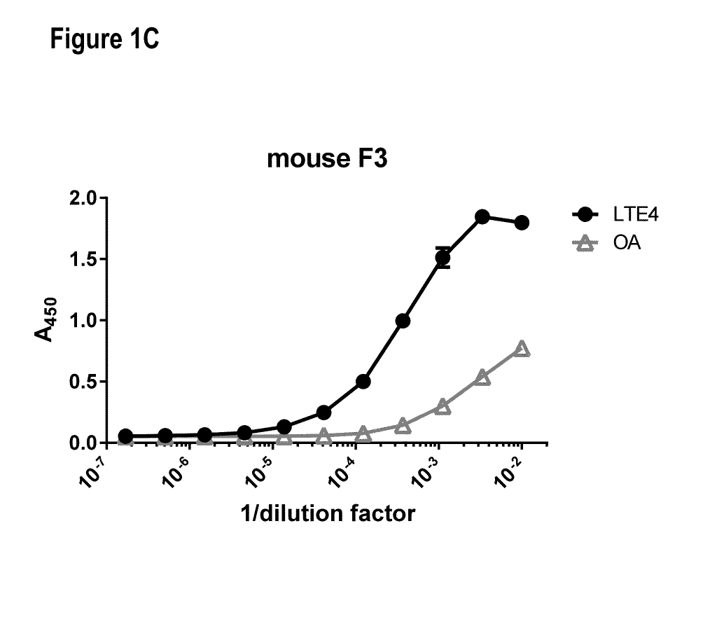

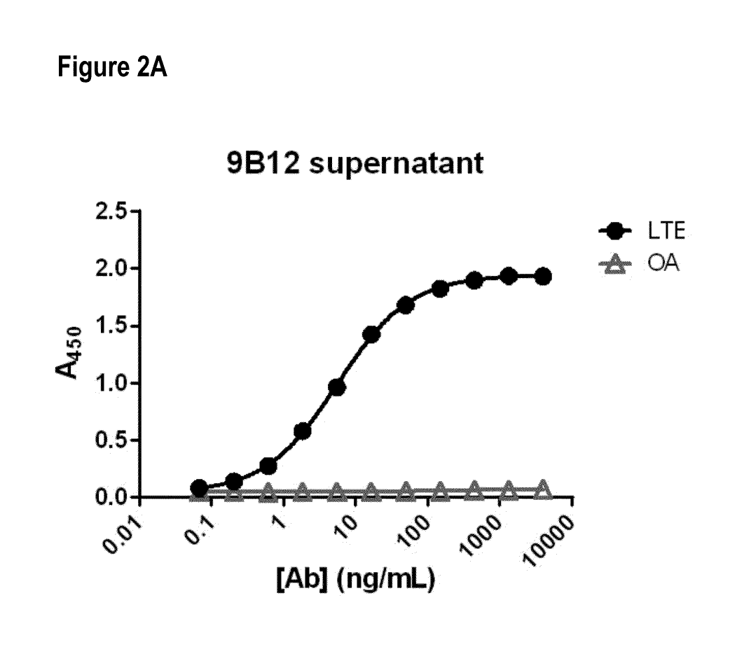

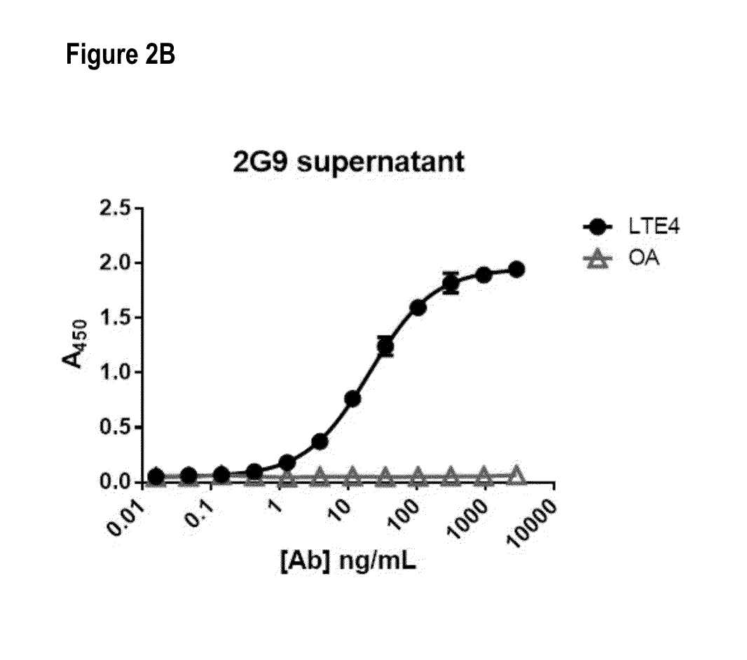

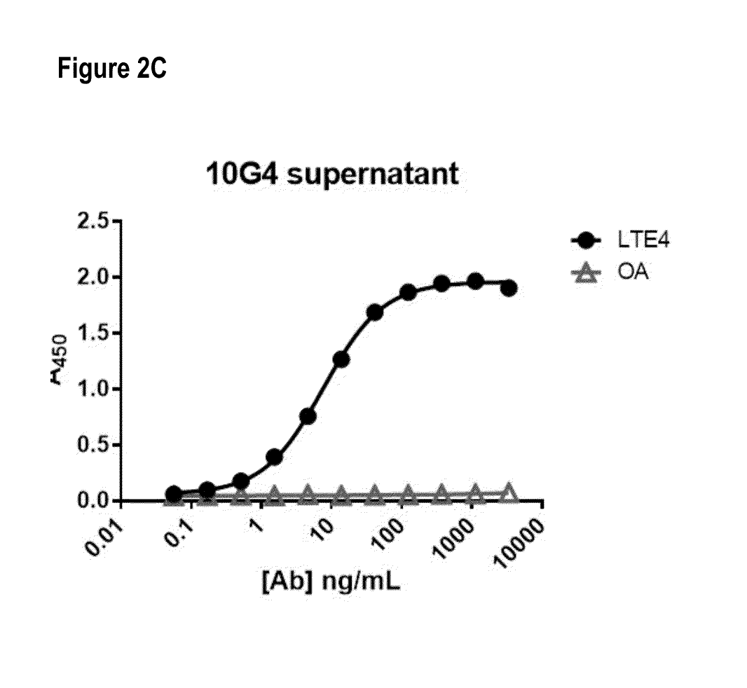

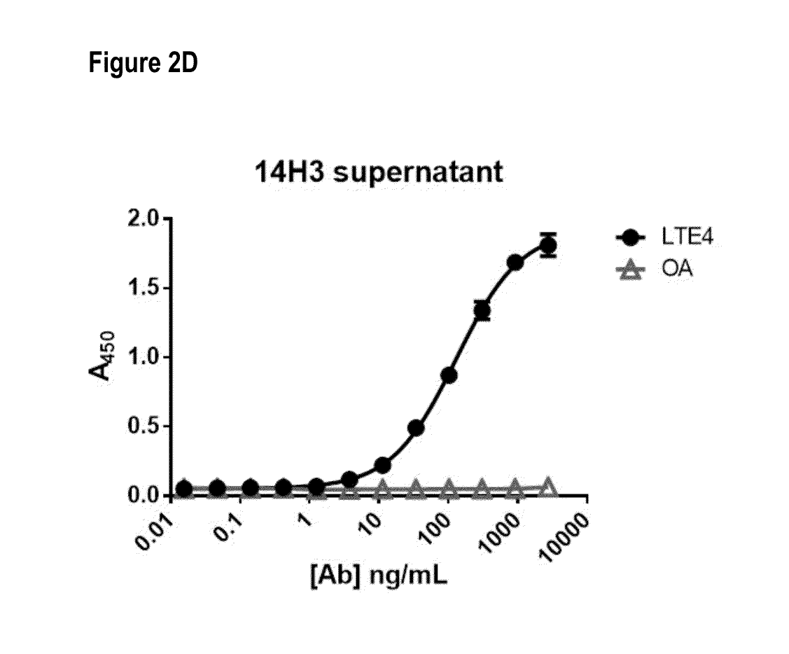

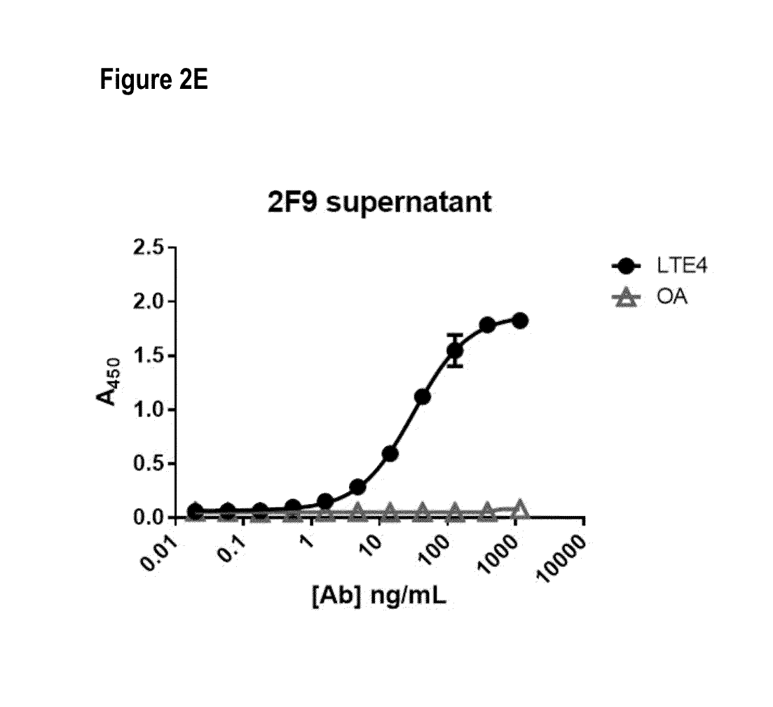

Nine 6-8-week old female Swiss Webster mice were immunized by two subcutaneous injections of 0.025 mg (0.05 mg total) of the immunogen (BS3 facilitated conjugate of LTE4 and BCP) emulsified in complete Freund's adjuvant. After 21 days, the mice were boosted with a single intraperitoneal (IP) injection of 0.05 mg of immunogen emulsified in incomplete Freund's adjuvant (IFA). Every week thereafter the mice received a single IP injection of 0.05 mg of immunogen emulsified in IFA for an additional 8 weeks. Serum samples were collected 3 days after the second, third, fifth, and ninth boosts and screened by direct ELISA as described below for the presence of anti-LTE4 antibodies (FIG. 1). Spleens from mice that displayed high antibody titers were subsequently used to generate hybridomas using the ClonaCell®-HY hybridoma cloning kit (Stemcell Technologies, Cat #03800). Once the hybridomas were grown to confluency, the cell supernatants were collected for ELISA analysis (FIG. 2).

### Example 3

### ELISA Screening

Serum and cell supernatants were screened for antibodies with LTE4-specific binding properties using the direct ELISA. An antigen-specific protein-lipid conjugate consisting of bovine serum albumin (BSA; Thermo Scientific, Cat #77110) crosslinked to LTE4 and an antigen-nonspecific protein-lipid conjugate consisting of BSA crosslinked to oleylamine (OA; Sigma, Cat #07805) were prepared, both using bis(succinimidyl)penta(ethylene glycol) (BSPEG5, Thermo Scientific, Cat #21581) as linker. Samples of interest (serum or supernatant) were applied to adjacent wells in 384-well high binding plates (Greiner Bio-One, Cat #781061) coated with 0.015 ug of either the antigen-specific or antigen-nonspecific conjugate, incubated for 1 h and washed off with PBS. The bound IgG was detected using a goat anti-mouse IgG1-specific HRP-conjugated antibody (Southern Biotech, Cat #1030-05) and developed with tetramethylbenzidine (TMB; Invitrogen, Cat #5B02). This colorimetric assay is read at A₄₅₀ (absorbance at a wavelength of 450 nm) on a plate reader, with higher A₄₅₀ indicating more antibody in the serum or supernatant sample. Samples that showed high signal on the LTE4-coated wells (antigen-specific) and no signal above background on the OA-coated wells (nonspecific) were deemed positive for antigen-specific binding properties (Table 1). As will be appreciated, screening with LTE4 as part of a conjugate that is distinct from that used as immunogen (both linker and protein differ) avoids false positives that would result from antibodies binding to the linker or protein portion of the immunogen rather than the lipid itself.

TABLE 1
ELISA Screen-binding signals for anti-cysLT serum
bleeds and hybridoma supernatants

| | 3^{rd} bleed titer | | | |
|---|---|---|---|---|
| | 1:2700 | | Supernatant | Supernatant |
| Mouse | A₄₅₀ | Hybridoma | A₄₅₀ | A₄₅₀ |
| ID | (LTE4) | ID | (LTE4) | (OA) |
| F4 | 1.151 | 9B12 | 1.425 | 0.073 |
| E2 | 0.858 | 10G4 | 1.254 | 0.063 |
| F3 | 0.997 | 2F9 | 1.525 | 0.054 |
| | | 2G9 | 1.512 | 0.051 |
| | | 14H3 | 1.257 | 0.057 |

After three fusions, supernatants from five hybridomas (9B12, 10G4, 2F9, 2G9, 14H3) were confirmed to show high affinity binding to all three cysLT (LTC4, LTD4, and LTE4) using the Kinetic Exclusion Assay (KinExA, Sapidyne Instruments, Boise Id.) (Table 2), and these were subjected to two rounds of limiting dilution (0.3 cells per well) cloning. In each round, 6-8 subclones were subjected to ELISA analysis. All the subclones were found to be positive for producing anti-cysLT antibodies. All the antibodies were isotyped as IgG1 kappa. Dissociation constants for these antibodies are shown in Table 2.

TABLE 2
Equilibrium dissociation constants for anti-cvsLT antibodies

| Ab | Lipid | Kd (pM) | 95% CI (pM) |
|---|---|---|---|
| 9B12 | LTE4 | 440 | 110-950 |
| | LTC4 | 4000 | 2100-6500 |
| | LTD4 | 630 | 240-1300 |
| 10G4 | LTE4 | 1500 | 1100-1900 |
| | LTC4 | 1.6 | <0.005-27 |
| | LTD4 | 140 | 75-210 |
| 2F9 | LTE4 | 38 | <0.139-83 |
| | LTC4 | 1886 | 1340-2545 |
| | LTD4 | 345 | 191-550 |
| 2G9 | LTE4 | 66 | 6-139 |
| | LTC4 | 2201 | 1201-3558 |
| | LTD4 | 867 | 23-2000 |
| 14H3 | LTE4 | 346 | 249-465 |
| | LTC4 | 2198 | 1715-2763 |
| | LTD4 | 4080 | 3240-5050 |

### Example 4

### Antibody Specificity

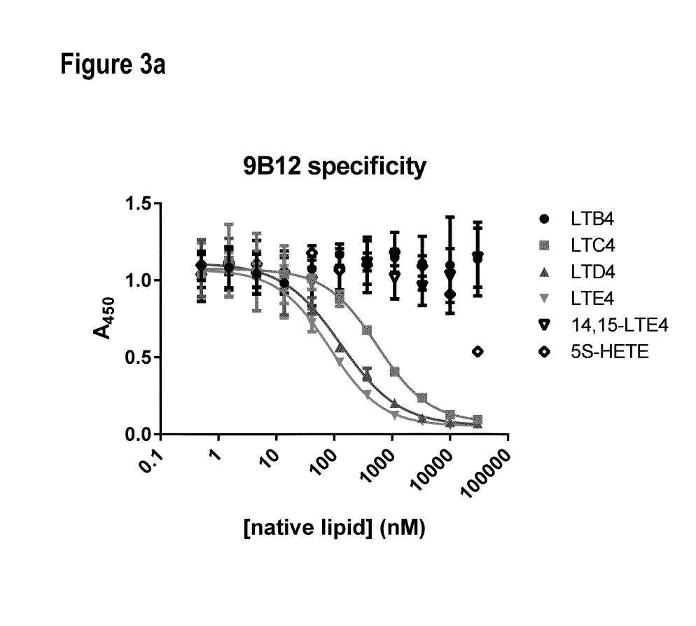

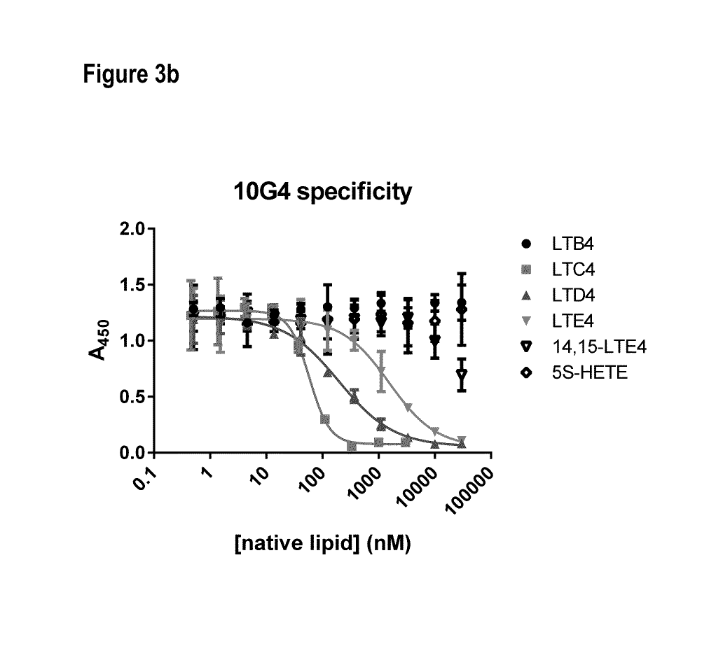

Monoclonal antibodies 9B12 and 10G4 were assayed for their binding specificity to a panel of cysLTs (LTC4, LTD4, and LTE4) as well as LTB4, 14,15-LTE4, and 5S-HETE. Briefly, 384-well high-binding microtiter plates were coated with 15 uL of the LTE4-PEG5-BSA conjugate (same conjugate used in the direct ELISA) diluted to a final concentration of 1 ug/mL using carbonate buffer, pH 9.5. After incubating the plates for 1 hour at 37° C., the plates were washed 4 times with PBS and blocked by adding 50 uL of 1% BSA (Calbiochem, Cat #126575), 0.1% Tween-20 in PBS to each well and incubating for 1 hour at room temperature. During this period, solutions containing 50 ng/mL of either 9B12 or 10G4 in 0.1 mg/mL BSA in PBS were prepared and used to titrate (3-fold serial dilutions) 30 micromolar solutions of the following native leukotriene lipids: LTB4 (Cayman Chemical Company, Cat #20110), LTC4 (Cayman Chemical Company, Cat #20210), LTD4 (Cayman Chemical Company, Cat #20310), LTE4 (Cayman Chemical Company, Cat #20410), 14,15-LTE4 (Cayman Chemical Company, Cat #10011362), and 5S-HETE (Cayman Chemical Company, Cat #34230). After removing the blocking solution and washing the plates 4× with PBS, 15 uL of the titration samples were pipetted into duplicate wells on the microtiter plates and incubated at room temperature for 3 hours. Following incubation, the antibody-lipid samples were removed and the plates were washed 4× with PBS. The IgG that remained bound to the immobilized conjugate was detected using a goat anti-mouse IgG1-specific HRP-conjugated antibody (Southern Biotech, Cat #1030-05), developed with tetramethylbenzidine (TMB; Invitrogen, Cat # S1302) and read at A₄₅₀. The results of these competition ELISAs are shown in FIG. 3.

FIG. 3a shows that antibody 9B12 binds to LTC4, LTD4, and LTE4, but not to LTB4, 14,15-LTE4, or 5S-HETE. With regard to LTC4, LTD4, and LTE4, the three are bound fairly similarly by the antibody (15%, 65%, and 100%, respectively). FIG. 3b shows that antibody 10G4 also binds to LTC4, LTD4, and LTE4 (100%, 29%, and 4%, respectively), but not to LTB4, 14,15-LTE4, or 5S-HETE. Antibodies 2F9, 2G9 and 14H3 were tested in the same manner and all three bound LTC4, LTD4 and LTE4 but with different specificity patterns [2F9: LTE4 binding>LTD4 binding>LTC4 binding (100%/18%/6%); 2G9: LTE4 binding>LTD4 binding>LTC4 binding (100%/37%/21%); 14H3: LTE4 binding>LTD4 binding>LTC4 binding (100%/68%/42%)]. None of 2F9, 2G9 or 14H3 bound LTB4, 14,15-LTE4 or 5S-HETE.

It can be seen from this example that cysLT monoclonal antibodies have been developed that bind preferentially to one or two cysLTs, or that bind well to all three cysLTs. Antibodies that bind preferentially to LTE4 or to LTC4 (such as 2F9 or 10G4, respectively) may be preferred in some instances; likewise a pan-cysLT antibody may be preferred in other instances. In yet other instances an antibody that binds preferentially to two cysLTs (for example antibody 9B12) may be preferred. These preferences may apply depending, for example, on the disease or condition to be treated, or when used for purposes of detection, on whether one, two or all three cysLTs are to be detected.

### Example 5

### Antibody Amino Acid Sequences

Cloning of the Murine mAbs:

A clone from each of the anti-cysLT hybridoma cell lines 9B12:3 H10 and 10G4:1 G2 was first grown with Stemcell's Medium E, then transferred to Iscove's DMEM (Corning Cellgro, Tewksbury Mass.) plus Gibco FBS & Cellgro supplements (no penicillin/streptomycin). For serum-free conditions, clones were grown in SFM4MAb-utility (Thermo Fisher Hyclone, Waltham Mass.) plus Cellgro supplements (no penicillin/streptomycin). Total RNA was isolated from 5E6 hybridoma cells using a procedure based on the NucleoSpin RNA kit (Macherey-Nagel, Bethlehem Pa.). mRNA was isolated from total RNA using oligo dT(25) magnetic beads (New England Biolabs, Ipswich Mass.). The mRNA was used to generate first strand cDNA followed by TdT tailing and PCR amplification, following the manufacturer's protocol for 5′RACE cloning (Invitrogen, Carlsbad Calif.).

The hybridoma subclones were shown to be of the mouse IgG1k isotype by testing culture supernatants with isostrips (Roche, Indianapolis Ind.). The immunoglobulin heavy chain variable region (VH) cDNA was generated using an isotype specific primer [RACEMOG1: 5′-TATGCAAGGCTTACAACCACA-3′ (SEQ ID NO: 1)]. The TdT-tailed cDNA was PCR amplified using a 5′ anchor primer [AAP: 5′-GGCCACGCGTCGACTAGTACGGGIIGGGIIGGGIIG-3′(SEQ ID NO: 2)] with a nested 3′ primer [MOCG1: 5′-CACAATTTTCTTGTCCACCTTGGTGC-3′ (SEQ ID NO: 3)]. The product of the reaction was purified using a NucleoSpin Gel and PCR clean-up kit (Macherey-Nagel) and sequenced using a reverse primer [CHIg-rev: 5′-CCTTGACCAGGCATCCCA-3′(SEQ ID NO: 4)]. The variable domain of the heavy chain was then amplified and inserted as a Age I and Afe I fragment and ligated into the expression vector pFUSE-CHIg-mG1 (Invivogen, San Diego Calif.) containing the hEF1-HTLV promoter, and the gamma-1 constant region to generate the plasmids pFUSE-10G4-mG1 and pFUSE-9B12-mG1.

Similarly, the immunoglobulin kappa chain variable region (VK) was amplified using an isotype specific primer [RACEMOCK 5′-CTCATTCCTGTTGAAGCTCTTGACAAT-3′ (SEQ ID NO: 5)]. The TdT-tailed cDNA was PCR amplified using the same 5′ anchor primer as for the VH, plus a kappa chain nested 3′ primer [CKMO: 5′-CTCATTCCTGTTGAAGCTCTTGACAATGGG-3′(SEQ ID NO: 6)]. The product of the reaction was purified using a NucleoSpin Gel and PCR clean-up kit (Macherey-Nagel) and sequenced using a reverse primer [CLIg-rev: 5′-AGTTGTTCAAGAAGCACACGA-3′(SEQ ID NO: 7)]. The variable domain of the light chain was then amplified and inserted as a Age I and BstAP I fragment and ligated into the expression vector pFUSE2-CLIg-mK (Invivogen) containing the hEF1-HTLV promoter, and the kappa constant region to generate the plasmids pFUSE2-10G4-mK and pFUSE-9B12-mK. The inserts were sequenced by Retrogen Inc., San Diego Calif.

The deduced CDR amino acid sequences of antibodies 9B12, 10G4, 2F9, 2G9 and 14H3 are shown in Tables 3-7. The variable domain sequences of these antibodies are shown in Tables 8-12. Leaders and other sequences such as cut sites that are not part of a variable domain itself but may be found surrounding the variable domain sequence (e.g., in a vector) are not shown. It should be noted that antibodies 2F9 and 2G9 share the same heavy chain sequence.

TABLE 3
CDR amino acid sequences of the mouse V_{H} and V_{L}
domains for clone 9B12 of mouse anti-cysLT
monoclonal antibody

| CLONE | V_{H} CDR | CDR | SEQ ID NO: |
|---|---|---|---|
| 9B12 | GYTFTDYYIH* | CDRH1 | 8 |
| 9B12 | RVNPNNGGTRYNQKFED | CDRH2 | 9 |
| 9B12 | SPLYYYDGRSGY | CDRH3 | 10 |
| | V_{L} CDR | | |
| 9B12 | RASSDVRYMY | CDRL1 | 11 |
| 9B12 | YTSNLAS | CDRL2 | 12 |
| 9B12 | QQFTTSPWT | CDRL3 | 13 |

*The CDRH1 sequence defined according to Oxford Molecular's (now Accelrys Inc., San Diego CA) AbM antibody modelling software is the 10-amino acid sequence shown (this CDRH1 sequence also matches the explanation by Dr. Andrew C.R. Martin of how to identify CDRs, found at http://www.bioinf.org.uk/abs/). The five-amino acid portion of this sequence shown in bold (DYYIH; SEQ ID NO: 14) is the CDRH1 sequence defined according to Kabat. The seven-amino acid portion of this sequence shown underlined (GYTFTDY; SEQ ID NO: 15) is the CDRH1 sequence defined according to Chothia.

TABLE 4
CDR amino acid sequences of the mouse V_{H} and V_{L}
domains for clone 10G4 of mouse anti-cysLT
monoclonal antibody

| CLONE | V_{H} CDR | CDR | SEQ ID NO: |
|---|---|---|---|
| 10G4 | GYSITSSYSWN* | CDRH1 | 16 |
| 10G4 | NIYYSGSTNYNPSLKS | CDRH2 | 17 |
| 10G4 | PRV | CDRH3 | 18 |
| | V_{L} CDR | | |
| 10G4 | RASQEISGYLG | CDRL1 | 19 |
| 10G4 | AASTLDS | CDRL2 | 20 |
| 10G4 | LQYASFPRT | CDRL3 | 21 |

*The CDRH1 sequence defined according to Chothia/AbM is the 11-amino acid sequence shown. The six-amino acid portion of this sequence shown in bold (SSYSWN; SEQ ID NO: 22) is the CDRH1 sequence defined according to Kabat. The eight-amino acid portion of this sequence shown underlined (GYSITSSY; SEQ ID NO: 23) is the CDRH1 sequence defined according to Chothia.

TABLE 5
CDR amino acid sequences of the mouse V_{H} and V_{L}
domains for clone 2F9 of mouse anti-cysLT mono-
clonal antibody

| CLONE | V_{H} CDR | CDR | SEQ ID NO: |
|---|---|---|---|
| 2F9 | GYIFTNYWMH | CDRH1 | 24 |
| 2F9 | RIHPSDSDTDYNQKFKG | CDRH2 | 25 |
| 2F9 | TLKWDVGY | CDRH3 | 26 |
| | V_{L} CDR | | |
| 2F9 | SASSSINSTY | CDRL1 | 27 |
| 2F9 | RTSTLAS | CDRL2 | 28 |
| 2F9 | QQWSSYPLT | CDRL3 | 29 |

TABLE 6
CDR amino acid sequences of the mouse V_{H} and V_{L}
domains for clone 2G9 of mouse anti-cysLT mono-
clonal antibody

| CLONE | V_{H} CDR | CDR | SEQ ID NO: |
|---|---|---|---|
| 2G9 | GYIFTNYWMH | CDRH1 | 24 |
| 2G9 | RIHPSDSDTDYNQKFKG | CDRH2 | 25 |
| 2G9 | TLKWDVGY | CDRH3 | 26 |
| | V_{L} CDR | | |
| 2G9 | SASSSINSMY | CDRL1 | 30 |
| 2G9 | RTSTLAS | CDRL2 | 28 |
| 2G9 | QQWSSYPLT | CDRL3 | 29 |

TABLE 7
CDR amino acid sequences of the mouse V_{H} and V_{L}
domains for clone 14H3 of mouse anti-cysLT mono-
clonal antibody

| CLONE | V_{H} CDR | CDR | SEQ ID NO: |
|---|---|---|---|
| 14H3 | GYTFTSYWMH | CDRH1 | 31 |
| 14H3 | RILPSNSDTIYNQKFKD | CDRH2 | 32 |
| 14H3 | TLNWDVGY | CDRH3 | 33 |
| | V_{L} CDR | | |
| 14H3 | SASSSVSSMY | CDRL1 | 34 |
| 14H3 | RTSKLAS | CDRL2 | 35 |
| 14H3 | QQWSSNPLT | CDRL3 | 36 |

It can be seen from comparison of the CDR sequences of these five monoclonal antibodies that the four antibodies that bind preferentially to LTE4, particularly antibodies 2F9, 2G9 and 14H3, share significant sequence identity in each of their CDRs, while the CDRs of antibody 10G4, which binds preferentially to LTC4, are less similar. This is illustrated in Table 8 below, in which each of the six CDR sequences in these five antibodies are compared (using 2G9 sequences as reference).

TABLE 8
Percent identities among cysLT monoclonal anti-
body CDR sequences

| | | SEQ | |
|---|---|---|---|
| | | ID | % identity |
| Antibody | CDRH1 sequence | NO | to 2G9 |
| 10G4 | GYSITSSYSWN | 16 | 30% |
| 9B12 | GYTFTDYYIH | 8 | 60% |
| 2F9 | GYIFTNYWMH | 24 | 100% |
| 2G9 | GYIFTNYWMH | 24 | 100% |
| 14H3 | GYTFTSYWMH | 31 | 80% |
| Consensus | GY*FT*Y**H | 37 | |
| excluding 10G4 | | | |
| | CDRH2 sequence | | |
| 10G4 | NIYYSGSTNYNPSLKS | 17 | 18% |
| 9B12 | RVNPNNGGTRYNQKFED | 9 | 41% |
| 2F9 | RIHPSDSDTDYNQKFKG | 25 | 100% |
| 2G9 | RIHPSDSDTDYNQKFKG | 25 | 100% |
| 14H3 | RILPSNSDTIYNQKFKD | 32 | 76% |
| Consensus | R**P****T*YNQKF** | 38 | |
| excluding 10G4 | | | |
| | CDRH3 sequence | | |
| 10G4 | PRV | 18 | N/A |
| 9B12 | SPLYYYDGRSGY | 10 | N/A |
| 2F9 | TLKWDVGY | 26 | 100% |
| 2G9 | TLKWDVGY | 26 | 100% |
| 14H3 | TLNWDVGY | 33 | 88% |
| Consensus | TL*WDVGY | 39 | |
| excluding 10G4 | | | |
| | CDRL1 sequence | | |
| 10G4 | RASQEISGYLG | 19 | 30% |
| 9B12 | RASSDVRYMY | 11 | 50% |
| 2F9 | SASSSINSTY | 27 | 90% |
| 2G9 | SASSSINSMY | 30 | 100% |
| 14H3 | SASSSVSSMY | 34 | 80% |
| Consensus | *ASS*****Y | 40 | |
| excluding 10G4 | | | |
| | CDRL2 sequence | | |
| 10G4 | AASTLDS | 20 | 57% |
| 9B12 | YTSNLAS | 12 | 71% |
| 2F9 | RTSTLAS | 28 | 100% |
| 2G9 | RTSTLAS | 28 | 100% |
| 14H3 | RTSKLAS | 35 | 86% |
| Consensus | *TS*LAS | 41 | |
| excluding 10G4 | | | |
| | CDRL3 sequence | | |
| 10G4 | LQYASFPRT | 21 | 44% |
| 9B12 | QQFTTSPWT | 13 | 44% |
| 2F9 | QQWSSYPLT | 29 | 100% |
| 2G9 | QQWSSYPLT | 29 | 100% |
| 14H3 | QQWSSNPLT | 36 | 89% |
| Consensus | QQ****P*T | 42 | |
| excluding 10G4 | | | |

Table 8 also shows a consensus sequence for each CDR, excluding those from antibody 10G4, demonstrating sites of identity among the amino acid sequences of each CDR from antibodies 9B12, 2F9, 2G9, 14H3 (*=position at which the amino acids are different among these antibodies). It can also be seen from Table 8 that the CDRs of antibodies 2F9, 2G9 and 14H3 are particularly similar, with at least 76% (76%-100%) sequence identity in the CDRs.

TABLE 9
Clone 9B12 variable domain amino acid sequences

| | |
|---|---|
| | SEQ ID |
| Sequence | NO: |

9B12 Heavy Chain

| | |
|---|---|
| EVQLQQSGPEMVKPGASVKISCKTSGYTFTDYYIHWVKQSHGKSLEWIGRVNP | 43 |
| NNGGTRYNQKFEDKATLTVDKSPSTAYMELNSLTSEDSAVYYCAISPLYYYDG | |
| RSGYWGQGTTLTVSS | |

9B12 Light Chain

| | |
|---|---|
| ENVLTQSPAILSATLGEKVTMSCRASSDVRYMYWHQQKSGASPKLWIYYTSNL | 44 |
| ASGVPARFSGSGSGTSYSLTISSVEAEDAATYYCQQFTTSPWTFGGGTKLEIK | |

TABLE 10
Clone 10G4 variable domain amino acid sequences

| | |
|---|---|
| Sequence | SEQ ID NO: |

10G4 Heavy Chain

| | |
|---|---|
| DVQLQESGPGLVKPSQSLSVTCTVTGYSITSSYSWNWIRQFPGNKLEWMGNIY | 45 |
| YSGSTNYNPSLKSRISITRDTSKNQFFLQLNSVTTEDTATYYCANPRVWGAGTT | |
| VTVSS | |

10G4 Light Chain

| | |
|---|---|
| DIQMTQSPSSLSASLGERVSLTCRASQEISGYLGWLQQKPDGTIKRLIYAASTLD | 46 |
| SGVPKRFSGSRSGSDYSLTISSLESEDFADYYCLQYASFPRTFGGGTKLEIQ | |

TABLE 11
Clone 2F9 variable domain amino acid sequences

| | |
|---|---|
| Sequence | SEQ ID NO: |

2F9 Heavy Chain

| | |
|---|---|
| QVQLQQPGAELVKPGASLRVSCRASGYIFTNYWM HWVKQRPGQGLEWIGRIH | 47 |
| PSDSDTDYNQKFKGKATLTVDKSSSTVYMQLSSLTSADSAVYYCATTLKWDVG | |
| YWGQGTTLTVSS | |

2F9 Light Chain

| | |
|---|---|
| QIVLTQSPTIMSASPGEKVTMTCSASSSINSTYWYQQKPGSSPKPWIYRTSTLA | 48 |
| SGVPVRFSGSGSGTSYSLTISSMEAEDAATYYCQQWSSYPLTFGAGTKLEMK | |

TABLE 12
Clone 2G9 variable domain amino acid sequences

| | |
|---|---|
| Sequence | SEQ ID NO: |

2G9 Heavy Chain

| | |
|---|---|
| QVQLQQPGAELVKPGASLRVSCRASGYIFTNYWMHWVKQRPGQGLEWIGRIH | 47 |
| PSDSDTDYNQKFKGKATLTVDKSSSTVYMQLSSLTSADSAVYYCATTLKWDVG | |
| YWGQGTTLTVSS | |

2G9 Light Chain

| | |
|---|---|
| QIVLTQSPTIMSASPGEKVTMTCSASSSINSMYWYQQKPGSSPKPWIYRTSTLA | 49 |
| SGVPVRFSGSGSGTSYSLTISSMEAEDAATYYCQQWSSYPLTFGAGTKLEMK | |

TABLE 13
Clone 14H3 variable domain amino acid sequences

| | |
|---|---|
| Sequence | SEQ ID NO: |

14H3 Heavy Chain

| | |
|---|---|
| QVQLQQPGAELVKPGASVKVSCKTSGYTFTSYWMHWVKQRPGQGLEWIGRIL | 50 |
| PSNSDTIYNQKFKDKATLTVDKSSSTVYMQLTSLTSEDSAVYYCAITLNWDVGY | |
| WGQGTTLTVSS | |

14H3 Light Chain

| | |
|---|---|
| QIVLTQSPAIMSASPGEKVTMTCSASSSVSSMYWYQQKPGSSPRPWICRTSKL | 51 |
| ASGVPVRFSGSGSGTSYSLTISSMEAEDAATYYCQQWSSNPLTFGAGTKLEMK | |

### Example 6

### Effect of Anti-cysLT Antibodies on Vascular Permeability

Airway (particularly trachea and bronchiole) inflammation is a hallmark of asthma. In addition to inflammatory cell infiltration in the bronchial wall, structural and functional changes related to the vasculature in the airway occur. These include the proliferation of blood vessels (angiogenesis), increased blood flow, increased microvascular permeability and edema formation in the airway wall. These vascular changes are correlated with to asthma severity, including airflow limitation and bronchial hyperresponsiveness and thus are clinically important. Horvath and Wanner (2006), Eur. Respir. J. 27:172-187. Each of the cysLTs is known to increase vascular permeability. Lee, et al. (2009), J. Allergy. Clin. Immunol. 124:417-421.

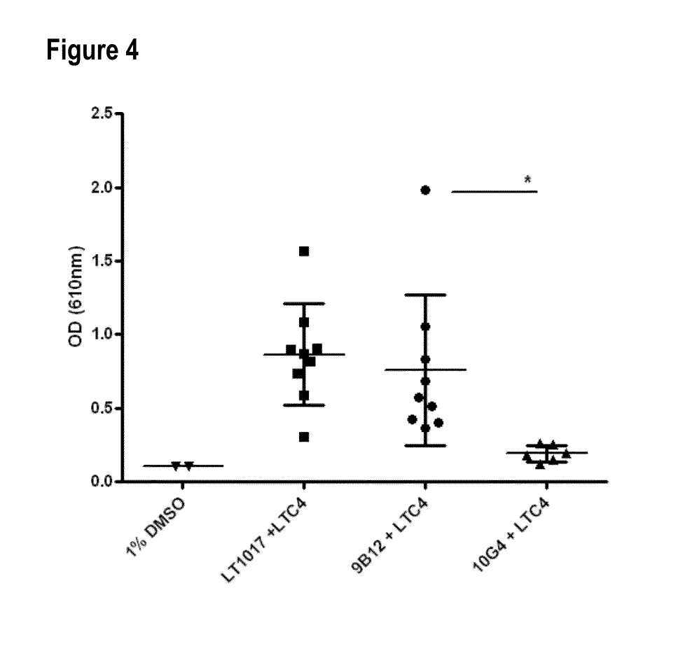

Vascular permeability was assessed in a preliminary study using standard methods. Briefly, Mice were injected intravenously with Evan's blue dye before intraperitoneal injection of 1.5 nanomoles each of LTC4 and antibody (isotype control LT1017 or anti-cysLT antibody (9B12 or 10G4) which had been preincubated together overnight at 4° C. After 15 min, mice were anaesthetized and peritoneal lavages were obtained. After centrifugation at 400×g for 10 min, the OD610 of the lavage supernatants was read using a spectrophotometer to determine how much blue dye had extravasated from the vasculature. As shown in FIG. 4, there was no leakage of dye into the peritoneal cavity when animals were injected i.p. with vehicle alone (1% DMSO). When LTC4 was injected along with unrelated control antibody there was demonstrable extravasation of dye. In contrast, when LTC4 was injected along with anti-cysLT antibody 10G4, dye leakage was minimal, indicating that the anti-cys antibody neutralized the effects of LTC4 on vascular permeability in this study. Results with anti-cysLT antibody 9B12 were unclear in this preliminary experiment. It should be noted that antibody 9B12 has a lower affinity for LTC4 than does antibody 10G4.

### Example 7

### Effect of Anti-cysLT Antibodies in Ovalbumin-Induced Acute Asthma

Antibodies 9B12 and 10G4 are tested in a model of acute asthma as described by Wu, et al. (2003), Clin. & Exp. Allergy 33:359-366.

Six- to 8-week-old BALB/c mice are separated into three treatment groups. Saline and antibody groups are immunized by intraperitoneal (i.p.) injections of 50 pg of OVA (Sigma, St. Louis Mo.) and 100 μL of alum (Pierce Thermo Fisher Scientific, Rockford Ill.) on days 0, 7 and 14. On day 21, the mice are placed in a 10×18×25 cm polypropylene chamber and challenged once with a nebulized solution of OVA (10 mg/mL) for 30 min. In the negative control group, OVA with alum is injected on days 0 and 7, saline is injected on day 14, and the animals are challenged with nebulized saline on day 21.

To determine the dose-response, various doses of antibody or saline are injected into the caudal tail vein daily from day 19-23 and the animals killed 72 h after OVA challenge. Bronchoalveolar lavage (BAL) fluid differential cell counts and lung histology are performed on these animals.

For the rest of the study, animals are dosed with antibody or saline intravenously (i.v.) from days 19 to 21. Twenty-four hours after OVA challenge, the mice are killed by i.p. injection of 0.2 mL sodium pentobarbital (60 mg/kg). The lungs are lavaged through the trachea with 1.2 mL of saline. A differential count of at least 200 cells is performed.

It is expected that antibodies to cysLT reduce the number of inflammatory cells in the BAL fluid after OVA challenge.

### Example 8

### Effect of Anti-cysLT Antibodies in Ovalbumin-Induced Chronic Asthma

Antibodies 9B12 and 10G4 are tested in a model of chronic asthma (Temelkovski, et al. (1998), Thorax 53: 849-856).

Sensitization: Pathogen-free female BALB/c mice aged 8-10 weeks are either sensitized by inhalational exposure to ovalbumin without prior systemic immunization or receive an intraperitoneal injection of 10 μg of alum precipitated chicken egg ovalbumin (grade V, >98% pure, Sigma, St Louis, Mo.) 21 days before inhalational exposure and a booster injection seven days before inhalational exposure (“boosted” mice).

Inhalation exposure: Mice are exposed to aerosolised ovalbumin for 30 min/day on three days/week for up to eight weeks with assessment of responses usually at intervals of two weeks. Experimental groups comprise six mice at each time point. Exposures are carried out in a whole body inhalation exposure system (Unifab Corp., Kalamazoo, Mich.). During the exposure the animals are held in wire flow-through cage racks and filtered air is drawn through the 0.5 m3 inhalation chamber at a flow rate of 2501/min. Temperature and relative humidity are maintained at 20-25° C. and 40-60%, respectively. A solution of 2.5% ovalbumin in normal saline is aerosolized by delivery of compressed air to a sidestream jet nebulizer and injected into the airstream prior to entering the chamber.

Treatment: Measurements of airway reactivity to intravenously administered antibodies to cysLT are performed 48 hours after the last inhalational exposure. A bronchospasm transducer [Ugo Basil 7020; Ugo Basile, Varese, Italy)] is used to determine airway constriction during cumulative intravenous administration of antibody at various doses to anesthetized mice ventilated under constant pressure. For each animal the increase in respiratory overflow volume provoked by antibody treatment is represented as a percentage of maximal overflow obtained by occluding the tracheal cannula. For comparison between treatment groups, these dose-response data are used to calculate the concentration that produced a decrease below baseline in airway occlusion (lung resistance). Control groups for these studies are mice that are sham immunized with adjuvant alone as well as boosted mice, both exposed to aerosolized normal saline.

### Example 9

### Effect of Anti-cysLT Antibodies in AERD

PGE2 synthase-1-null mice (Uematsu, et al. (2002), J Immunol 168:5811-5816) develop a remarkably AERD-like phenotype in a model of eosinophilic pulmonary inflammation. Mice lacking mPGES-1 (ptges−/− mice) treated intranasally (i.n.) with an extract from the dust mite Dermatophagoides farina (Df) develop marked eosinophilic bronchovascular inflammation compared with wild-type control animals (Liu, 2012). Df-treated ptges−/− mice exhibit cysLT production and cysLT-dependent airflow obstruction and lung mast cell activation in response to aspirin. Liu, 2013. Lung resistance is assessed with an invasive pulmonary function device (Buxco, Wilmington N.C.). Briefly, mice are tracheostomized and ventilated. After allowing lung resistance to reach a stable baseline, Lys-aspirin (Lys-ASA), 12 μL of 100 mg/mL solution, or diluent alone, is delivered to the lung via nebulizer 24 h after their last doses of Df or saline, and lung resistance is recorded for 45 min. The results are expressed as percentage change of lung resistance from baseline. Lung resistance increases markedly in the Df-treated ptges−/− mice challenged with Lys-ASA compared with the WT mice and the saline-treated ptges−/− controls. This increase is expected to be reduced in animals treated with antibody to cysLT (9B12 or 10G4).

### Example 10

### Effect of Anti-cysLT Antibodies 2G9 and 10G4 and Various Dosing Methods on Vascular Permeability

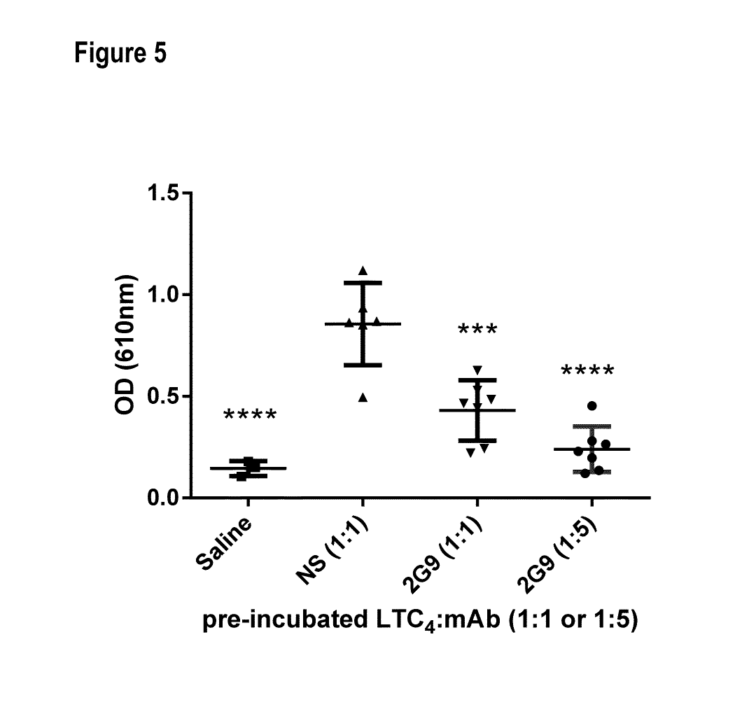

Vascular permeability was assessed in a preliminary study using standard methods, as in Example 6. Briefly, Mice were injected intravenously with Evan's blue dye before intraperitoneal injection of 1.5 nanomoles each of LTC4 (Cayman, Cat #20210) and antibody (isotype control LT1017 or anti-cysLT antibody 2G9) which had been preincubated together overnight at 4° C. After 15 min, mice were anesthetized and peritoneal lavages were obtained. After centrifugation at 400×g for 10 min, the OD610 of the lavage supernatants was read using a spectrophotometer to determine how much blue dye had extravasated from the vasculature. As shown in FIG. 5, there was no leakage of dye into the peritoneal cavity when animals were injected I.P. with saline alone. When LTC4 was injected along with unrelated control antibody (NS) at a LTC4:antibody molar ratio of 1:1, there was demonstrable extravasation of dye. In contrast, when LTC4 was injected along with anti-cysLT antibody 2G9 (LTC4:antibody ratio of 1:1 or 1:5), dye leakage was reduced in a dose-dependent manner, with the 1:5 mixture reducing extravasation to nearly control (saline) levels. This indicates that the anti-cys antibody 2G9 neutralized the effects of LTC4 on vascular permeability in this study. Asterisks indicate statistically significant difference when compared to nonspecific negative control (NS) antibody: ***P=0.0002, ****P<0.0001 statistically different from NS (1:1) group (one-way ANOVA with Dunnett's multiple comparison tests).

The effect of anti-cysLT antibody 10G4 was also evaluated in a “pre-dose” subcutaneous (SC) experiment in which 10G4 or nonspecific control antibody LT1017 (NS) was administered (30 mg/kg SC) to mice 24 hr prior to administration of LTC4 (1.5 nanomoles, I.P). This study also included a 10G4 IP group, in which the equivalent amount of antibody to a 30 mg/kg dose (˜0.6 mg or ˜4 nanomoles) was pre-mixed with 1.5 nanomoles of LTC4 and the mixture was injected I.P.

As in the previous study, administration of saline alone did not cause dye leakage.

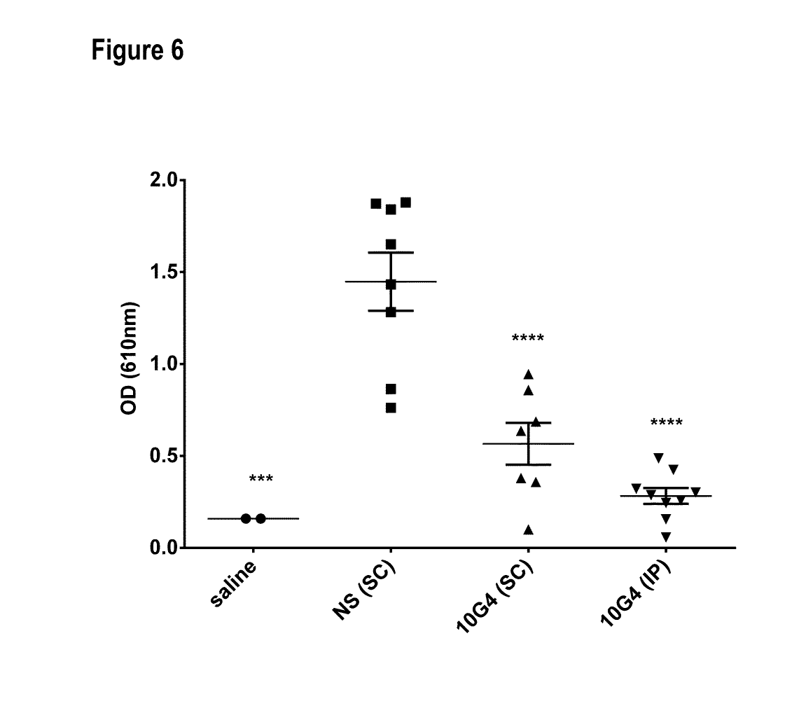

Subcutaneous nonspecific control antibody (NS) administration followed by LTC4 administration resulted in extravasation of dye. In contrast, extravasation (vascular permeability) caused by LTC4 was significantly reduced in animals pretreated subcutaneously with anti-cysLT antibody 10G4. This is shown in FIG. 6. Asterisks indicate statistically significant difference when compared to nonspecific negative control (NS) antibody: ****P<0.0001 statistically different from NS(SC) group (one-way ANOVA with Dunnett's multiple comparison tests).

This example indicates that anti cysLT antibodies were able to inhibit LTC4-induced vascular permeability, regardless of route of administration. In addition, this inhibition of vasopermeability was seen even when antibody was given 24 hr before LTC4.

### Example 11

### Evaluation of Pharmacokinetics (PK) of Anti-cysLT mAbs in Mice

Sixteen female BALB/c mice (6-8 weeks old) were administered a single 30 mg/kg bolus dose of anti-cysLT antibody (9B12, 2G9 or 10G4) by intraperitoneal (I.P.) or intravenous (I.V.) injection. The mice were randomized and divided into 4 groups (4 animals per group). At various times post-dose (1, 3, 6, 9, 24, 72, 144, 192, 336 and 504 hours), blood was collected from one group of mice (N=4) using either the superficial temporal vein or cardiac puncture technique. Plasma was isolated using the CAPIJECT capillary blood collection system (Terumo, Somerset N.J., Cat # T-MQK) with ETDA-containing tubes.

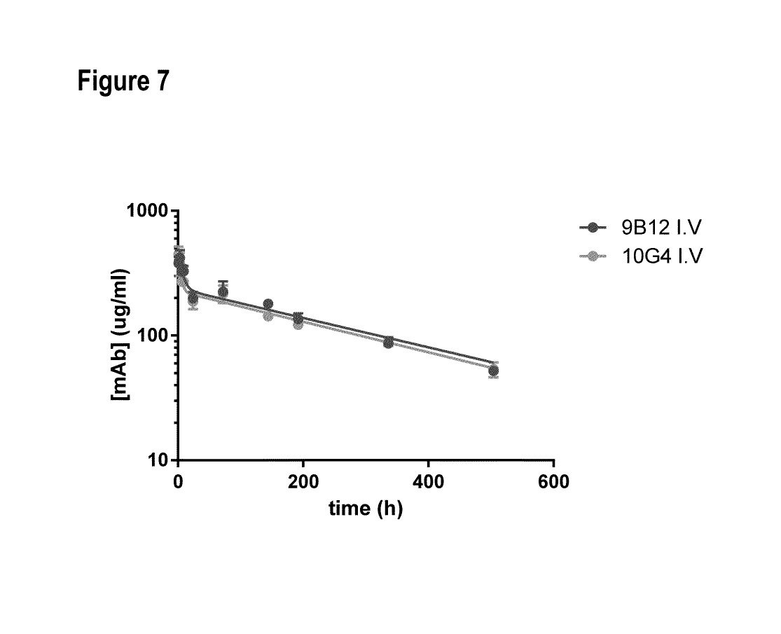

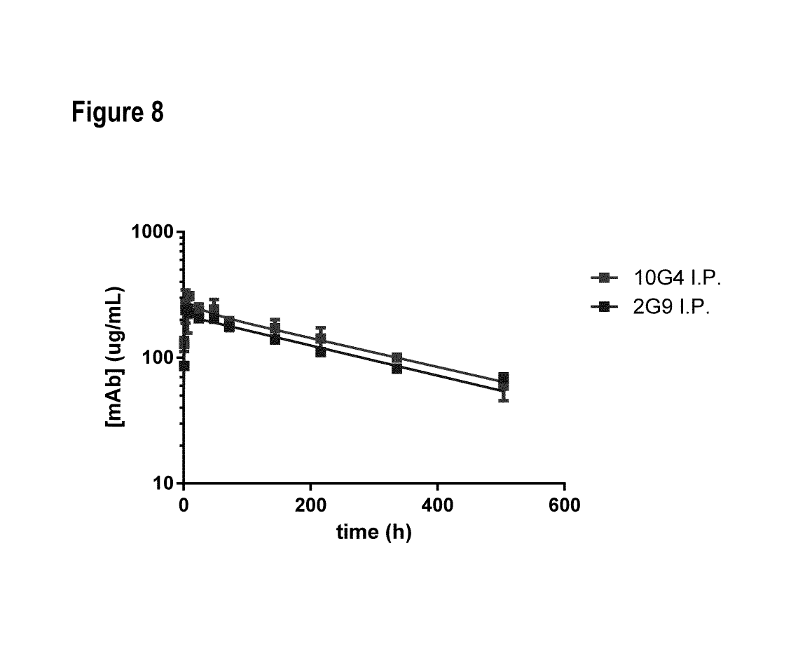

The anti-cysLT antibody levels in the isolated mouse plasma samples were quantified using the direct-binding ELISA. Briefly, plasma samples were diluted 1:100, 1:1000, 1:2000, 1:4000, 1:8000 and 1:16000 using blocking buffer (1×PBS, 10 mg/mL BSA, 0.05% tween-20), and 100 uL aliquots of each dilution were added in duplicate to 96-well ELISA plates (Greiner Bio-One, Monroe N.C., Cat #655061) previously coated with 0.1 ug of a LTE4-BSA conjugate and blocked with blocking buffer. After 1 hour incubation, the plates were washed and anti-cysLT antibody bound to the plate was detected using a goat anti-mouse IgG-HRP antibody (SouthernBiotech, Birmingham Ala., Cat #1030-05) and 3,3′,5,5′-tetramethylbenzidine (TMB) substrate (Invitrogen, San Diego Calif., Cat #5B02). The absorbance at 450 nM was measured and compared to a standard curve, generated using the purified antibody material, to determine the anti-cysLT antibody concentration in the plasma samples. The antibody concentrations were plotted vs. time post-dose and curve-fit using a 3-phase exponential function (FIGS. 7 and 8). As shown in FIG. 7, the pharmacokinetic profiles of murine anti-cysLT antibodies 9B12 and 10G4, both administered intravenously, are virtually identical.

As shown in FIG. 8, the pharmacokinetic profiles of murine anti-cysLT antibodies 10G4 and 2G9, both administered intraperitoneally, are also virtually identical. Comparison of 10G4 given IV (FIG. 7) and IP (FIG. 8) indicates that the pharmacokinetic profiles appear to be largely independent of route of administration.

### Example 12

### Humanization of Murine Antibody 10G4

The variable domains V_{H} and V_{L} of the murine anti-cysLT monoclonal antibody, 10G4, were humanized by grafting the murine CDRs into human framework regions (FR), with the goal of producing an antibody that retains high affinity, specificity and binding capacity for one or more cysLTs. Lefranc, M. P, (2003). Nucleic Acids Res, 31: 307-10; Martin, A. C. and J. M. Thornton, (1996) J Mol Biol, 1996. 263: 800-15; Morea, V., A. M. Lesk, and A. Tramontano (2000) Methods, 20: 267-79; Foote, J. and G. Winter, (1992) J Mol Biol, 224: 487-99; Chothia, C., et al., (1985). J Mol Biol, 186:651-63.

Suitable acceptor human framework sequences were selected using the IgBLAST free online tool from NIH's National Center for Biotechnology Information. Human immunoglobulin heavy variable 4-59 (accession no. AB019438) was selected as the human framework on which to base the humanized version of the 10G4 heavy chain variable domain and JH6 (accession no. J00256) was used for the heavy chain J region. The CDRs of the heavy chain were those of the murine antibody.

For the light chain, VKI O12 (accession no. X59315) was selected as the human framework on which to base the humanized version of the 10G4 light chain variable domain. JK2 (accession no. J00242) was used for the J region. The CDR sequences are those of the murine antibody 10G4.

The sequences of the first humanized version of the 10G4 antibody (10G4 CDRs grafted into human frameworks named above, no backmutations) are shown below in Tables 13 and 14. This is referred to as the 10G4 humanized antibody template.

The DNA and amino acid sequences of the heavy chain variable (VH) template (10G4 heavy chain CDRs in the human frameworks) are shown in Table 14. CDRs are in bold; sequences not coding for the variable domain (i.e., signal sequence, constant domain sequences) are underlined. Amino acid positions 6-24, inclusive, are the 4-59 human framework leader:

TABLE 14
The DNA and amino acid sequence of the humanized 10G4 VH template:

| | |
|---|---|
| aagcttgccgccaccatgaaacatctgtggttcttccttctcctggtggcagctcccaga | (SEQ ID NO: 53) |
| K L A A T M K H L W F F L L L V A A P R | (SEQ ID NO: 52) |
| tgggtcctgtcccaagtgcagttgcaggaatcaggcccaggcctggtgaaaccaagcgag | |
| W V L S Q V Q L Q E S G P G L V K P S E | |
| acactgagcttgacctgcactgtgtccggttactcaatcacctcctcttacagctggaac | |
| T L S L T C T V S G Y S I T S S Y S W N | |
| tggatcaggcagccacctggaaagggccttgagtggatcgggaatatctattactctggc | |
| W I R Q P P G K G L E W I G N I Y Y S G | |
| tccactaactataatccttccctgaaatccagggtgaccatttctgttgatacaagtaaa | |
| S T N Y N P S L K S R V T I S V D T S K | |
| aaccagttctctcttaaactttctagtgtgactgcagcagatacagcagtctattattgt | |
| N Q F G L K L S S V T A A D T A V Y Y C | |
| gcccgaccccgggtttggggccagggaaccactgtaaccgtttcttctgccagcaccaag | |
| A R P R V W G Q G T T V T V S S A S T K | |
| ggccc | |

The coding region of the amino acid sequence of the heavy chain variable domain above is as follows:

(SEQ ID NO: 54)
QVQLQESGPGLVKPSETLSLTCTVSGYSITSSYSWNWIRQPPGKGLEWIG
NIYYSGSTNYNPSLKSRVTISVDTSKNQFSLKLSSVTAADTAVYYCARPR
VWGQGTTVTVSS

The DNA and amino acid sequences of the VL template (10G4 light chain CDRs in the human frameworks) is shown in Table 15. CDRs are in bold and sequences not coding for the variable domain (i.e., signal sequence, constant domain sequences) are underlined. Amino acid positions 6-27, inclusive, are the human O12 framework leader sequence:

TABLE 15
The DNA and amino acid sequences of the humanized 10G4 variable
light chain (VL) template

| | |
|---|---|
| aagcttgccgccaccatggacatgagggtccccgctcagctcctggggctcctgctactc | (SEQ ID NO: 55) |
| K L A A T M D M R V P A Q L L G L L L L | (SEQ ID NO: 56) |
| tggctccgaggtgccagatgtgacatccagatgacacagtcaccatcttcccttagcgcc | |
| W L R G A R C D I Q M T Q S P S S L S A | |
| tctgttggcgaccgcgtcaccattacttgtagagctagccaggagatttctggctatctc | |
| S V G D R V T L T C R A S Q E I S G Y L | |
| ggctggtatcaacaaaaacccggtaaagctccaaagctgctcatctatgctgctagcact | |
| G W Y Q Q K P G K A P K L L I Y A A S T | |
| cttgactctggtgttccatctcgcttctcaggtagtgggtccgggactgatttcaccctc | |
| L D S G V P S R F S G S G S G T D F T L | |
| actatttctagcctgcagcctgaagacttcgccacttactattgcctgcagtacgcatct | |
| T I S S L Q P E D F A T Y Y C L Q Y A S | |
| Ttcccacggacatttggacagggcaccaaacttgagataaagcgtacg | |
| F P R T F G Q G T K L E I K R T | |

The coding region of the amino acid sequence of the light chain variable domain above is as follows:

(SEQ ID NO: 57)
DIQMTQSPSSLSASVGDRVTITCRASQEISGYLGWYQQKPGKAPKLLIYA
ASTLDSGVPSRFSGSGSGTDFTLTISSLQPEDFATYYCLQYASFPRTFGQ
GTKLEIK

The humanized antibody template comprising the above heavy and light chains (murine CDRs grafted into fully human frameworks) was expressed as full-length IgG antibody in HEK293 cells human embryonic kidney cell line (FreeStyle™ 293-F Cells, Life Technologies, Cat # R790-07, using FreeStyle™ 293 Expression Medium (Life Technologies, Cat #12338-018), and 293Fectin™ Transfection Reagent (Life Technologies, Cat #12347-019). After transient expression, supernatants were harvested and IgG was quantified by ELISA. Activity was tested by direct ELISA and the humanized template (murine CDRs in fully human frameworks) was found to be inactive in the absence of backmutations.

### Example 13

### Backmutations of Humanized 10G4-Optimization of Variants

A series of variants of the 10G4 humanized light chain variable region and heavy chain region were made. The heavy chain variants are shown in Table 16 and the light chain variants are shown in Table 17. Also shown are variants with differing combinations of multiple backmutations. It should be noted that two variants with CDR mutations were made: heavy chain variant 10G4/4-59.8 has a single backmutation and also a mutation (S30bG) in CDRH1, and light chain variant 10G4/O12.8 has four backmutations as well as two CDR mutations (E28S in CDRL1 and S93R in CDRL3).

TABLE 16
Heavy chain variable domain variants

| | VH Sequence (coding portion only). | | SEQ |
|---|---|---|---|
| Variant | CDRs are shown in bold | | ID |
| name | and backmutations are underlined | Backmutation | NO: |
| 10G4/4-59.0 | QVQLQESGPGLVKPSETLSLTCTVSGYSITSSYSWNWI | None (fully | 54 |
| (template) | RQPPGKGLEWIGNIYYSGSTNYNPSLKSRVTISVDTSK | human) | |
| | NQFSLKLSSVTAADTAVYYCARPRVWGQGTTVTVSS | | |
| 10G4/4-59.1 | QVQLQESGPGLVKPSETLSVTCTVSGYSITSSYSWNWI | L2OV | 58 |
| | RQPPGKGLEWIGNIYYSGSTNYNPSLKSRVTISVDTSK | | |
| | NQFSLKLSSVTAADTAVYYCARPRVWGQGTTVTVSS | | |
| 10G4/4-59.2 | QVQLQESGPGLVKPSETLSLTCTVSGYSITSSYSWNWI | P40F | 59 |
| | RQFPGKGLEWIGNIYYSGSTNYNPSLKSRVTISVDTSK | | |
| | NQFSLKLSSVTAADTAVYYCARPRVWGQGTTVTVSS | | |
| 10G4/4-59.3 | QVQLQESGPGLVKPSETLSLTCTVSGYSITSSYSWNWI | VT67/68IS | 60 |
| | RQPPGKGLEWIGNIYYSGSTNYNPSLKSRISISVDTSK | | |
| | NQFSLKLSSVTAADTAVYYCARPRVWGQGTTVTVSS | | |
| 10G4/4-59.4 | QVQLQESGPGLVKPSETLSLTCTVSGYSITSSYSWNWI | V71R | 61 |
| | RQPPGKGLEWIGNIYYSGSTNYNPSLKSRVTISRDTSK | | |
| | NQFSLKLSSVTAADTAVYYCARPRVWGQGTTVTVSS | | |
| 10G4/4-59.5 | QVQLQESGPGLVKPSETLSLTCTVSGYSITSSYSWNWI | R94N | 62 |
| | RQPPGKGLEWIGNIYYSGSTNYNPSLKSRVTISVDTSK | | |
| | NQFSLKLSSVTAADTAVYYCANPRVWGQGTTVTVSS | | |
| 10G4/4-59.6 | QVQLQESGPGLVKPSETLSVTCTVSGYSITSSYSWNWI | L20V, P40F, | 63 |
| | RQFPGKGLEWIGNIYYSGSTNYNPSLKSRISISRDTSK | VT67/68IS, | |
| | NQFSLKLSSVTAADTAVYYCANPRVWGQGTTVTVSS | V71R, R94N | |
| 10G4/4-59.7 | QVQLQESGPGLVKPSETLSVTCTVSGYSITSSYSWNWI | L20V, P40F, | 64 |
| | RQFPGKGLEWIGNIYYSGSTNYNPSLKSRVTISRDTSK | V71R, R94N | |
| | NQFSLKLSSVTAADTAVYYCANPRVWGQGTTVTVSS | | |
| 10G4/4-59.8 | QVQLQESGPGLVKPSETLSLTCTVSGYSITSGYSWNWI | R94N plus CDR | 65 |
| | RQPPGKGLEWIGNIYYSGSTNYNPSLKSRVTTSVDTSK | mutation S30bG | |
| | NQFSLKLSSVTAADTAVYYCANPRVWGQGTTVTVSS | | |

TABLE 17
Light chain variable domain variants

| | VL Sequence (coding portion only). | | SEQ |
|---|---|---|---|
| | CDRs are shown in bold and backmutations | | ID |
| Variant name | are underlined | Backmutation | NO: |
| 10G4/O12.0 | DIQMTQSPSSLSASVGDRVTITCRASQEISGYLGWY | None (fully human) | 57 |
| (template) | QQKPGKAPKLLIYAASTLDSGVPSRFSGSGSGTDFT | | |
| | LTISSLQPEDFATYYCLQYASFPRTFGQGTKLEIK | | |
| 10G4/O12.1 | DIQMTQSPSSLSASVGDRVTITCRASQEISGYLGWL | Y36L | 66 |
| | QQKPGKAPKLLIYAASTLDSGVPSRFSGSGSGTDFT | | |
| | LTISSLQPEDFATYYCLQYASFPRTFGQGTKLEIK | | |
| 10G4/O12.2 | DIQMTQSPSSLSASVGDRVTITCRASQEISGYLGWY | P44I | 67 |
| | QQKPGKAIKLLIYAASTLDSGVPSRFSGSGSGTDFT | | |
| | LTISSLQPEDFATYYCLQYASFPRTFGQGTKLEIK | | |
| 10G4/O12.3 | DIQMTQSPSSLSASVGDRVTITCRASQEISGYLGWY | L46R | 68 |
| | QQKPGKAPKRLIYAASTLDSGVPSRFSGSGSGTDFT | | |
| | LTISSLQPEDFATYYCLQYASFPRTFGQGTKLEIK | | |
| 10G4/O12.4 | DIQMTQSPSSLSASVGDRVTITCRASQEISGYLGWY | G66R | 69 |
| | QQKPGKAPKLLIYAASTLDSGVPSRFSGSRSGTDFT | | |
| | LTISSLQPEDFATYYCLQYASFPRTFGQGTKLEIK | | |
| 10G4/O12.5 | DIQMTQSPSSLSASVGDRVTITCRASQEISGYLGWL | Y36L, P44I, L46R, | 70 |
| | QQKPGKAIKRLIYAASTLDSGVPSRFSGSRSGTDFT | G66R | |
| | LTISSLQPEDFATYYCLQYASFPRTFGQGTKLEIK | | |
| 10G4/O12.6 | DIQMTQSPSSLSASVGDRVTITCRASQEISGYLGWL | Y36L, P44I, L46R, | 71 |
| | QQKPGKAIKRLIYAASTLDSGVPSRFSGSRSGTDYT | G66R, F71Y | |
| | LTISSLQPEDFATYYCLQYASFPRTFGQGTKLEIK | | |
| 10G4/O12.7 | DIQMTQSPSSLSASVGDRVTITCRASQEISGYLGWL | Y36L, L46R, G66R | 72 |
| | QQKPGKAPKRLIYAASTLDSGVPSRFSGSRSGTDFT | | |
| | LTISSLQPEDFATYYCLQYASFPRTFGQGTKLEIK | | |
| 10G4/O12.8 | DIQMTQSPSSLSASVGDRVTITCRASQSISGYLGWL | Y36L, P44I, L46R, | 73 |
| | QQKPGKAIKRLIYAASTLDSGVPSRFSGSRSGTDFT | G66R plus CDR | |
| | LTISSLQPEDFATYYCLQYARFPRTFGQGTKLEIK | mutations E28S and | |
| | | S93R | |

The variant variable domains in the tables above were cloned into separate vectors and transiently transfected in various heavy and light chain combinations into an HEK293 human embryonic kidney cell line (FreeStyle™ 293-F Cells, Life Technologies, Cat # R790-07, using FreeStyle™ 293 Expression Medium (Life Technologies, Cat #12338-018), and 293Fectin™ Transfection Reagent (Life Technologies, Cat #12347-019). After transient expression, supernatants were harvested and the IgG was quantified by ELISA. Binding activity of each variant for LTE4 (conjugated to BSA) was tested initially using direct ELISA. Variants that showed binding in the ELISA were further evaluated for binding native cyLTs using KinExA.

The combinations of heavy chains (HC) and light chains (LC) tested are shown in Table 18 below. The number of backmutations in the framework (FW) of each variable domain is indicated.

TABLE 18
CysLT humanized antibody variants

| Heavy | | Light | |
|---|---|---|---|
| chain | | chain | |
| (HC) | | (LC) | |
| variable | | variable | |
| domain | Backmutations | domain | Backmutations |
| Set 1 Multiple backmutations vs fully | | | |
| human (“all or none”) | | | |
| 4-59.0 | none | O12.0 | none |
| 4-59.0 | none | O12.5 | Y36L, P44I, L46R, G66R |
| 4-59.6 | L20V, P40F, VT67/68IS, | O12.0 | none |
| | V71R, R94N | | |
| 4-59.6 | L20V, P40F, VT67/68IS, | O12.5 | Y36L, P44I, L46R, G66R |
| | V71R, R94N | | |
| Set 2 Variants vs fully human | | | |
| 4-59.1 | L20V | O12.0 | none |
| 4-59.2 | P40F | O12.0 | none |
| 4-59.3 | VT67/68IS | O12.0 | none |
| 4-59.4 | V71R | O12.0 | none |
| 4-59.5 | R94N | O12.0 | none |
| 4-59.0 | none | O12.1 | Y36L |
| 4-59.0 | none | O12.2 | P44I |
| 4-59.0 | none | O12.3 | L46R |
| 4-59.0 | none | O12.4 | G66R |
| Set 3 | | | |
| 4-59.6 | L20V, P40F, VT67/68IS, | O12.1 | Y36L |
| | V71R, R94N | | |
| 4-59.6 | L20V, P40F, VT67/68IS, | O12.2 | P44I |
| | V71R, R94N | | |
| 4-59.6 | L20V, P40F, VT67/68IS, | O12.3 | L46R |
| | V71R, R94N | | |
| 4-59.6 | L20V, P40F, VT67/68IS, | O12.4 | G66R |
| | V71R, R94N | | |
| 4-59.1 | L20V | O12.5 | Y36L, P44I, L46R, G66R |
| 4-59.2 | P40F | O12.5 | Y36L, P44I, L46R, G66R |
| 4-59.3 | VT67/68IS | O12.5 | Y36L, P44I, L46R, G66R |
| 4-59.4 | V71R | O12.5 | Y36L, P44I, L46R, G66R |
| 4-59.5 | R94N | O12.5 | Y36L, P44I, L46R, G66R |
| Set 4 | | | |
| 4-59.1 | L20V | O12.1 | Y36L |
| 4-59.2 | P40F | O12.1 | Y36L |
| 4-59.3 | VT67/68IS | O12.1 | Y36L |
| 4-59.4 | V71R | O12.1 | Y36L |
| 4-59.5 | R94N | O12.1 | Y36L |
| 4-59.1 | L20V | O12.2 | P44I |
| 4-59.2 | P40F | O12.2 | P44I |
| 4-59.3 | VT67/68IS | O12.2 | P44I |
| 4-59.4 | V71R | O12.2 | P44I |
| 4-59.5 | R94N | O12.2 | P44I |
| 4-59.1 | L20V | O12.3 | L46R |
| 4-59.2 | P40F | O12.3 | L46R |
| 4-59.3 | VT67/68IS | O12.3 | L46R |
| 4-59.4 | V71R | O12.3 | L46R |
| 4-59.5 | R94N | O12.3 | L46R |
| 4-59.1 | L20V | O12.4 | G66R |
| 4-59.2 | P40F | O12.4 | G66R |
| 4-59.3 | VT67/68IS | O12.4 | G66R |
| 4-59.4 | V71R | O12.4 | G66R |
| 4-59.5 | R94N | O12.4 | G66R |

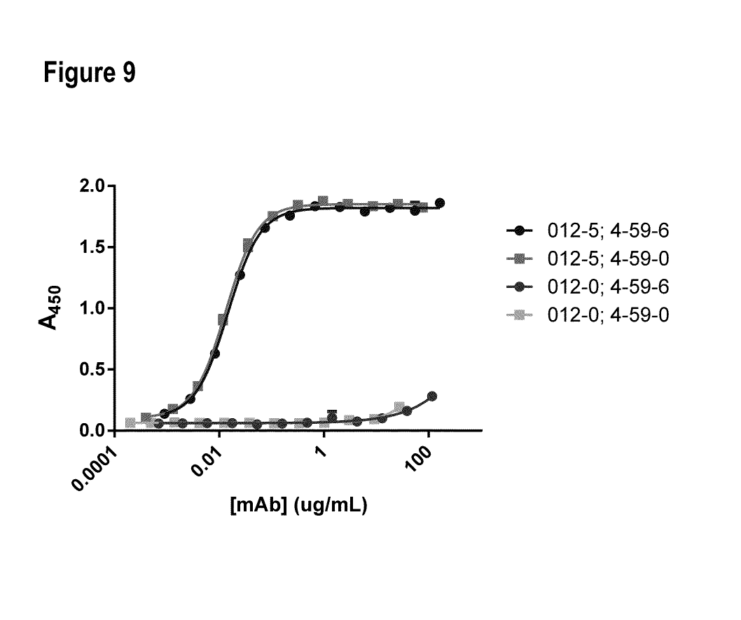

Initially an “all or none” screen (Set 1 in Table 18) was done, in which antibodies containing humanized variable domains having no backmutations (fully human frameworks), or multiple backmutations, were tested for ability to bind LTE4. FIG. 9 shows direct LTE4-binding ELISA data for humanized 10G4 variants from set 1, having no backmutations in either chain (light chain variable region O12.0 and heavy chain variable region 4-59.0), having multiple backmutations (light chain variable region O12.5 and heavy chain variable region 4-59.6) in both chains, or having one chain with no backmutations and one chain with multiple backmutations. It can be seen that antibodies having variant light chains containing no backmutations (O12.0) were inactive, regardless of heavy chain, so it is presently believed that one or more backmutations in the human light chain framework are needed for activity. However, humanized antibodies whose heavy chains contain no backmutations (5-59.0, lines in blue) were still able to bind antigen regardless of light chain framework mutations.

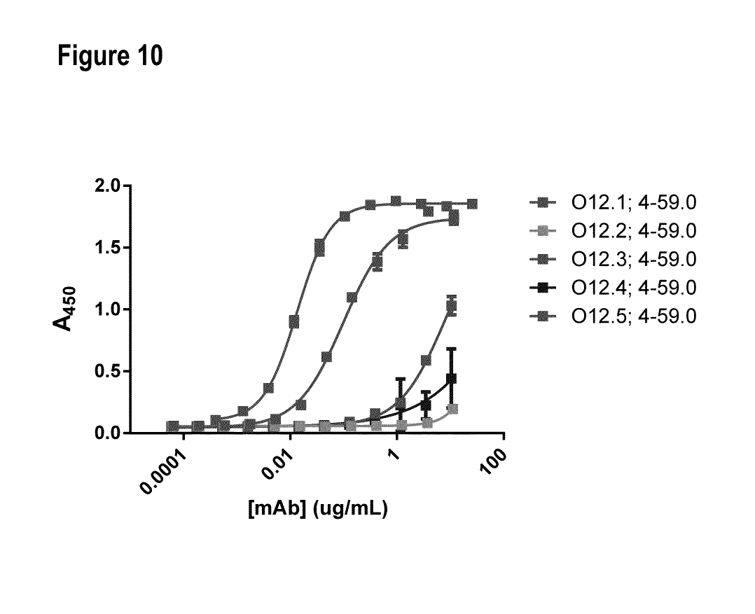

Humanized variants of Set 2 (Table 18) were also tested for LTE4 binding by direct ELISA. Light chain humanized variants each having a single backmutation in the variable domain were incorporated into antibodies along with heavy chain humanized variant 4-59.0, which is fully human (no backmutations). Included for comparison is the antibody from Set 1 having light chain variable domain O12.5 and heavy chain variable domain 4-59.0. As shown in FIG. 10, all of the antibodies showed some binding activity for LTE4, but the antibody with light chain variant O12.2 (single backmutation P441) showed the weakest binding. This backmutation was omitted in the second round of variants (see light chain variant O12.7 which lacks this mutation but contains the other three backmutations present in O12.5). When combined with fully human heavy chain variant 4-59.0 as in this experiment, the most active antibodies for LTE4 binding were those with light chains O12.5 and O12.1.

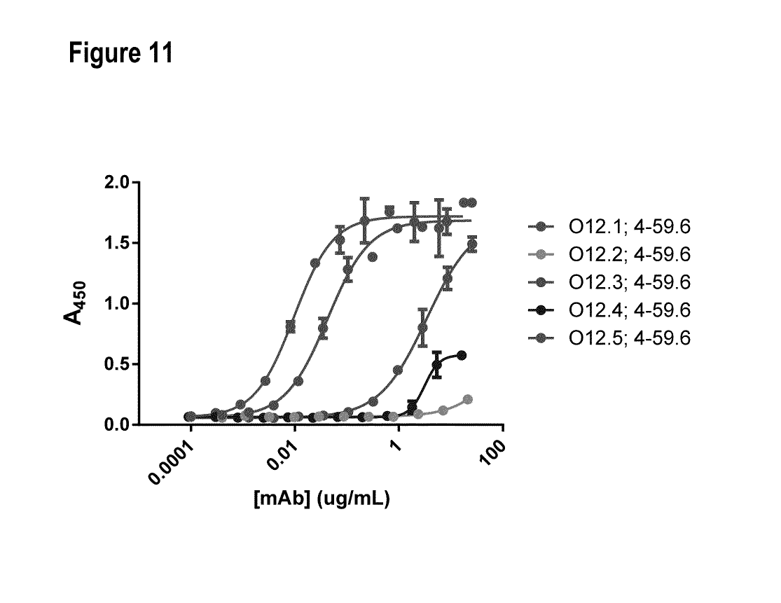

Humanized variants of Set 3 in Table 18 were tested for cysLT binding by direct ELISA using LTE4-BSA. Humanized 10G4 light chain variant variable domains with a single backmutation were expressed with the 4-59.6 heavy chain variable domain (six backmutations). The O12.5 light chain (four backmutations) was also expressed in combination with the 4-59.6 heavy chain. All of the antibodies showed some binding activity for LTE4, but again, antibodies containing a single light chain backmutation at position P44I (O12.2) again showed the weakest binding. This backmutation was omitted in the second round of variants (see light chain variant O12.7 which lacks this mutation but contains the other three backmutations present in O12.5). The most active antibodies for LTE4 binding in this screen were again those with light chains O12.5 and O12.1, as shown in FIG. 11.

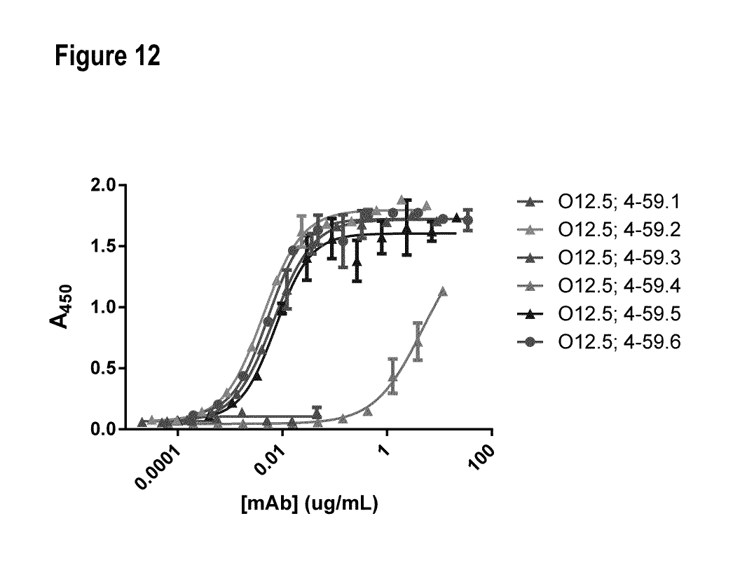

Next, humanized antibody 10G4 heavy chain variants 4-59.1, 4-59.2, 4-59.3, 4-59.4 and 4-59.5, each having one backmutation (or a pair of adjacent mutations in the case of 4-59.3), were expressed with light chain O12.5 (four backmutations) and tested by direct LTE4-binding ELISA. As shown in FIG. 12, the antibody with the 4-59.3 variant heavy chain (VT67/68IS backmutations) showed no detectable binding, those with heavy chain variants 4-59.1, 4-59.2 and 4-59.5 showed high binding activity, and the antibody with the 4-59.3 heavy chain showed intermediate binding activity for LTE4. An antibody with the O12.5 light chain variable region and the 4-59.6 heavy chain variable region (six backmutations) also showed excellent LTE4 binding activity.

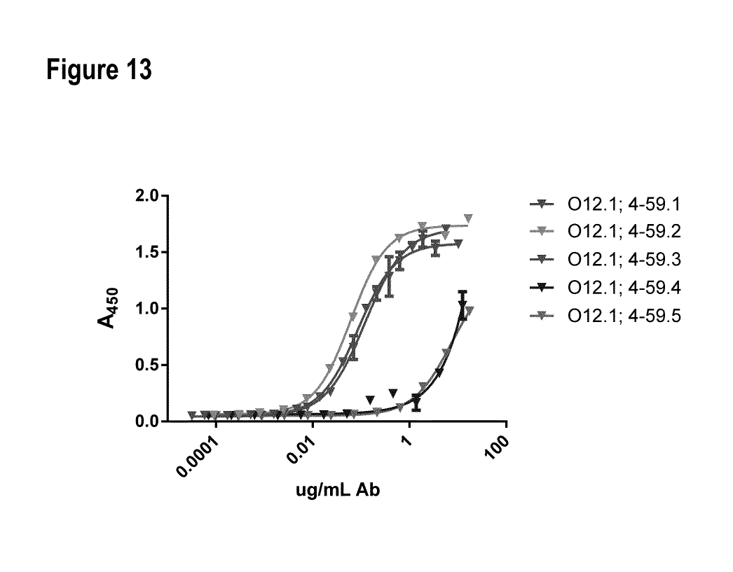

Finally, humanized antibody 10G4 variants from Set 4 (Table 18) were tested, in which heavy chain and light chain variants, each with a single mutation (or a pair of adjacent backmutations in the case of heavy chain 4-59.3) were expressed together in various combinations and the resulting humanized antibodies were tested by ELISA for LTE4 binding activity. FIG. 13 shows LTE4 binding of antibodies having the O12.1 light chain variable domain and different heavy chain variable domains (4-59.1, 4-59.2, 4-59.3, 4-59.4 or 4-59.5). Three antibodies in this series (having heavy chains 4-59.1, 4-59.2 and 4-59.3) showed high binding activity for LTE4. The remaining antibodies showed modest binding. This is consistent with the finding above that antibodies with light chain O12.1 tended to be among the most active.

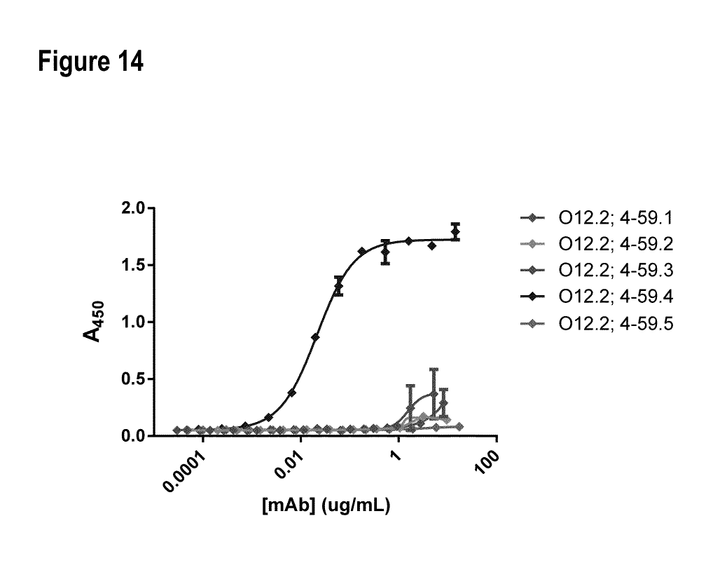

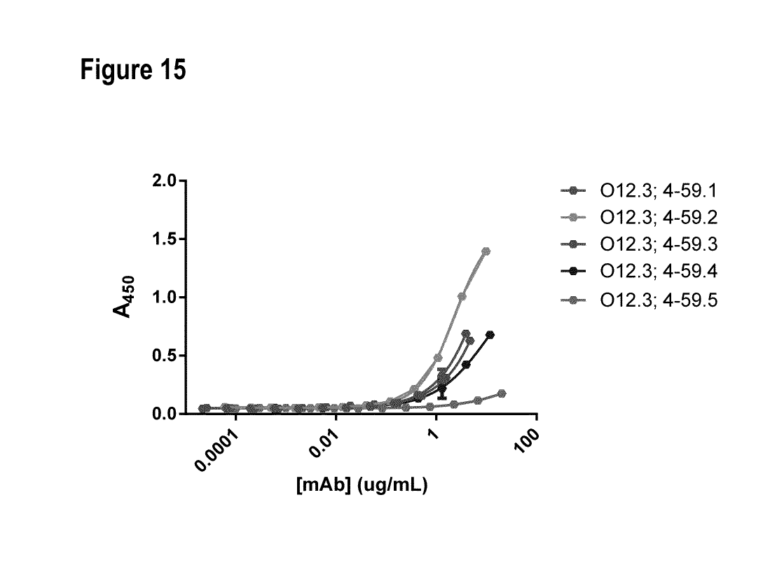

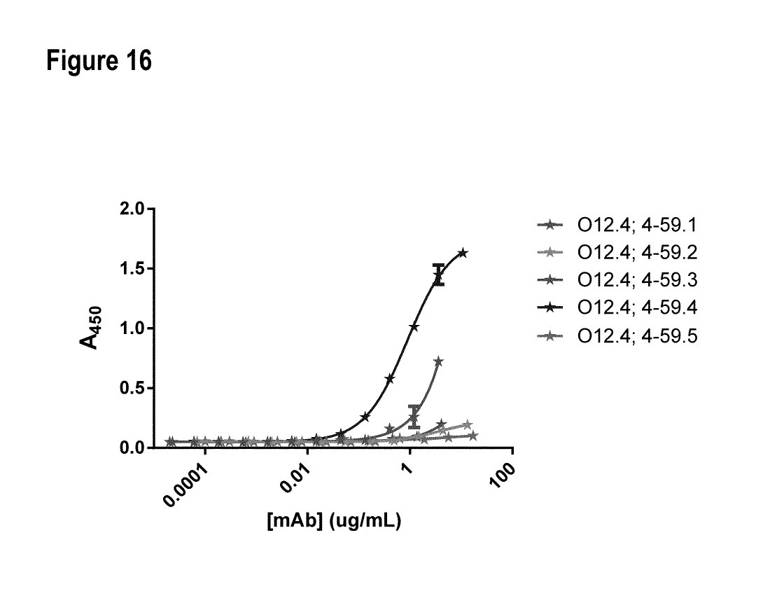

FIG. 14 shows LTE4 binding of antibodies having the O12.2 light chain variable domain and different heavy chain variable domains. In this series, only one antibody (4-59.4 heavy chain) had high binding activity and the remaining antibodies showed minimal binding. FIG. 15 shows LTE4 binding of antibodies having the O12.3 light chain variable domain and different heavy chain variable domains. The antibody with the 4-59.2 heavy chain showed the best binding in this series, the antibody with the 4-59.5 heavy chain showed minimal binding and the remainder were intermediate in binding.

FIG. 15 shows LTE4 binding of antibodies having the O12.4 light chain variable domain and different heavy chain variable domains. The antibody with the 4-59.4 heavy chain showed good binding, the 4-59.1 antibody showed modest binding and the remainder showed minimal binding.

### Example 14

### Additional Optimized Humanized Variants of Murine cysLT Antibody 10G4

Based on the data above, additional heavy and light chain variable domain variants (sequences shown in Tables 15 and 16 above) were generated with the heavy and light chain variable domains as shown in Table 19 below. Backmutations showing little to no binding activity in previous screens were omitted from the new variants. Light chain backmutation P441 was omitted in the second round of variants below (new light chain variant O12.7 lacks this mutation but contains the other three backmutations present in active variant O12.5). Heavy chain backmutations VT67 and 68OS were also omitted in the second round of variants below (new heavy chain variant 4-59.7 lacks these mutations but contains backmutations L20V, P40F, V71R, R94N present in 4-59.6).

Variable domain sequences are shown in Tables 15 and 16 above, and a comparison of the binding ability of antibodies containing these additional variant sequences with the variants generated as described in the previous example is shown in Table 20.

Binding of antibodies to native LTC4, LTD4 and LTE4 was determined by using the Kinetic Exclusion Assay (KinExA, Sapidyne Instruments, Boise Id.).

TABLE 19
Humanized antibody variants and the number of framework
backmutations and CDR mutations in each

| | VL × VH | No. of backmutations | | | CDR mutations | | |
|---|---|---|---|---|---|---|---|
| Variant | plasmids | LC | HC | Total | LC | HC | Total |
| LT5000 | murine 10G4 | n/a | n/a | n/a | 0 | 0 | 0 |
| LT5010 | O12.5 × 4-59.5 | 4 | 1 | 5 | 0 | 0 | 0 |
| LT5011 | O12.5 × 4-59.6 | 4 | 6 | 10 | 0 | 0 | 0 |
| LT5012 | O12.6 × 4-59.6 | 5 | 6 | 11 | 0 | 0 | 0 |
| LT5013 | O12.7 × 4-59.6 | 3 | 6 | 9 | 0 | 0 | 0 |
| LT5014 | O12.7 × 4-59.7 | 3 | 4 | 7 | 0 | 0 | 0 |
| LT5015 | O12.8 × 4-59.8 | 4 | 1 | 5 | 2 | 1 | 3 |

TABLE 20
Binding of variants to native LTC4,
LTD4 and LTE4 (by KinExA assay)

| | LTC4 | | LTD4 | | LTE4 | |
|---|---|---|---|---|---|---|
| | Kd | | Kd | | Kd | |
| Variant | (pM) | 95 CI | (pM) | 95 CI | (pM) | 95 CI |
| LT5000 | 5.4 | 7.2-3.9 | 65 | 90-44 | 560 | 660-470 |
| LT5011 | 1.5 | 4.3-<0.01 | 101 | 140-67 | 300 | 370-220 |
| LT5012 | 2.4 | 4.4-1.0 | 63 | 110-29 | 330 | 480-140 |
| LT5013 | 1.5 | 4.0-0.02 | 28 | 69-4.3 | 61 | 96-38 |
| LT5014 | 3.1 | 5.8-1 | 28 | 52-6.6 | 108 | 146-73 |

As can be seen from the above tables, antibodies LT5013 and LT5014 were highly active and were comparable in binding profile. LT5013 (light chain O12.7, heavy chain 4-59.6) has a total of 9 backmutations and LT5014 (light chain O12.7, heavy chain 4-59.7), has a total of 7 backmutations, respectively. LT5014 was chosen for further study.

The antibody LT5015, containing a total of three CDR mutations (CDRH1: GYSITSGYSWN, SEQ ID NO: 74; CDRL1, RASQSISGYLGW, SEQ ID NO; 75; AND CDRL3, LQYARFPRT, SEQ ID NO: 76) and five framework backmutations, was compared to LT5010 (same backmutations, no CDR mutations). This antibody showed good cysLT binding in direct ELISAs but was not found to have improved binding activity for native cysLTs compared to LT5010.

### Example 15

### Activity of Murine Anti-cysLT Antibodies 2G9 and 10G4 in a Murine Model of Inflammatory Bowel Disease

The dextran sulfate sodium-induced colitis (DSS-colitis) model is a widely accepted animal model for inflammatory bowel disease [see, for example, Deguchi et al. (2006) Oncology Reports 16:699-703]. The murine cys-LT antibodies 2G9 and 10G4 were evaluated in this model. Mice (10/group) were given drinking water containing 1.5% dextran sodium sulfate (DSS) for six days starting on day 0 of study, followed by clean water for four days. Antibody (30 mg/kg in PBS) was given on days 0, 3, and 6 of study. The body weight of each animal, stool consistency and the presence or absence of occult or gross blood in stool were monitored each day. From these a composite disease activity index (DAI) score was calculated. A DAI of zero is normal (no weight loss, no blood in stool, normal stool consistency) and the maximum DAI score is four (>15% weight loss, diarrhea, gross blood in stool). Cyclosporine A was used as a positive control and LT1014 was the nonspecific control antibody. Control mice received no DSS.

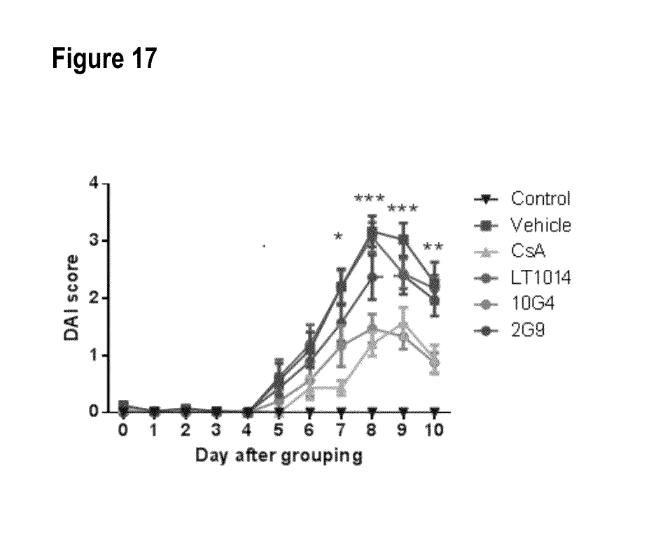

As seen in FIG. 17, control mice had no disease, as expected. Vehicle and nonspecific antibody (LT1014) treated groups had the highest DAI, and anti-cysLT antibody 10G4 and positive control cyclosporine A (CsA)-treated animals had the lowest DAI. Statistical significance for 10G4 vs vehicle is shown (* P<0.05, ** P<0.01, *** P<0.001) based on multiple t-tests.

### Example 16

### Activity of Murine Anti-cysLT Antibodies 9B12 and 10G4 in Murine Model of Allergic Asthma

Anti cysLT antibodies were evaluated in the OVA model of acute asthma by PharmaLegacy Laboratories, Shanghai, China, with specific regard to improvement of airway hyper-responsiveness, lung inflammation and lung histopathology.

Reagents: Ovalbumin (OVA): grade V, Sigma, St Louis Mo. Cat: A5503; Imject Alum hydroxide solution: Pierce, Rockford Ill., Cat: 77161; Sodium carboxymethyl cellulose (CMC, MW=800-1200), Sinopharm Chemical Reagent Co., Ltd, Shanghai, China, Cat: 30036365;

Phosphate buffered saline (PBS): Dycent Biotech (Shanghai) CO., Ltd. Cat: BJ141.

Groups: Female BALB/c mice were randomly grouped by body weight, 10 mice per group: Control (Sham sensitized, PBS vehicle only, i.v.); Model (PBS vehicle only, i.v.); Dexamethasone (SPGC Sine Pharma Laboratories) in 0.5% CMC-Na at 0.1 mg/mL, orally administered (positive control); 9B12 (30 mg/kg, i.v.) in PBS; 10G4 group (30 mg/kg, i.v.) in PBS. Mice in the control group received only 100 μL PBS (PH=7.2) by i.p. injection. Mice in all other groups were sensitized by injection (0.1 mL/mouse, i.p.) of sensitizing solution (containing 20 pg ovalbumin and 2 mg alum in PBS) on days 1 and 14.

OVA Challenge: On day 28, 29, 30, mice (except control group) were challenged with 1% OVA in PBS (PH=7.2) (challenge solution) for 30 min with mass dosing system (Buxco/DSI, St. Paul Minn.). Mice in the control group were challenged with PBS (PH=7.2).

Dosing:

Control group: vehicle was dosed intravenously on days 27, 29 (2 hours before OVA challenge), and 31 (2 hours before airway hyperresponsiveness (AHR) test).

Model group: vehicle was administered intravenously on days 27, 29 (2 hours before OVA challenge), and 31 (2 hours before airway hyperresponsiveness (AHR) test.

Dexamethasone positive control: (1.0 mg/kg) in 0.5% CMC-Na was administered orally once daily on days 27, 28, 29, 30 (2 hours before OVA challenge on each of challenge days) and 31.

LT1017: nonspecific antibody control was dosed intravenously on days 27, 29 (2 hours before OVA challenge), and 31 (2 hours before airway hyperresponsiveness (AHR) test).

Antibody 9B12: antibody was dosed intravenously on days 27, 29 (2 hours before OVA challenge), and 31 (2 hours before airway hyperresponsiveness (AHR) test).

Antibody 10G4: antibody was dosed intravenously on days 27, 29 (2 hours before OVA challenge), and 31 (2 hours before airway hyperresponsiveness (AHR) test).

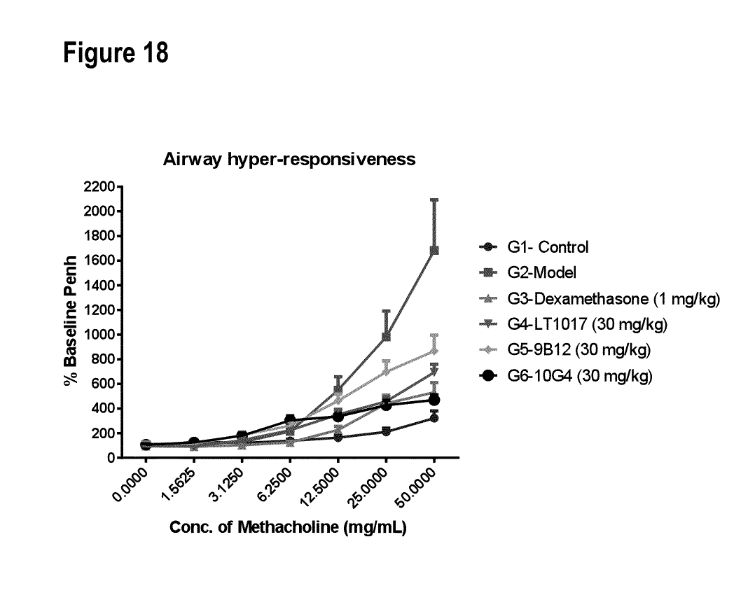

Airway hyper-responsiveness: On day 31 (24 hours after the last challenge), airway hyperresponsiveness, measured by “enhanced pause” or “penh” compared to baseline, was determined for all animals via whole body plethysmograph (Buxco), the mice were given aerosolized normal PBS (PH=7.2), followed by 1.5625, 3.125, 6.25, 12.5, 25, 50 mg/mL methacholine challenge, given serially. The results are shown in FIG. 18. As expected, it can be seen that the mice in which asthma was induced showed the highest penh and mice given no OVA showed the lowest penh. Anti-cysLT antibody 10G4 lowered the penh to roughly that of the positive control, dexamethasone. Antibody 9B12 and nonspecific control antibody lowered the penh to an intermediate level. Data are shown as mean±SEM.

Bronchoalveolar lavage and differential cell count: On day 32, all animals were anesthetized by intraperitoneal injection of 1% pentobarbital sodium (60 mg/kg). A blood sample was collected by retro-orbital bleeding, and plasma was isolated using EDTA. Lungs were lavaged via the tracheal cannula with 0.5 mL of PBS (PH=7.2) (containing 1% FBS) the first time. Then the course was repeated twice with 0.5 mL PBS (PH=7.2) (containing 1% FBS) for each time. All lavage fluid was pooled together. Cells were re-suspended in 1.5 mL PBS (PH=7.2) (containing 1% FBS) for cell number counting. Total numbers of cells in BAL fluid were counted by hemocytometer.

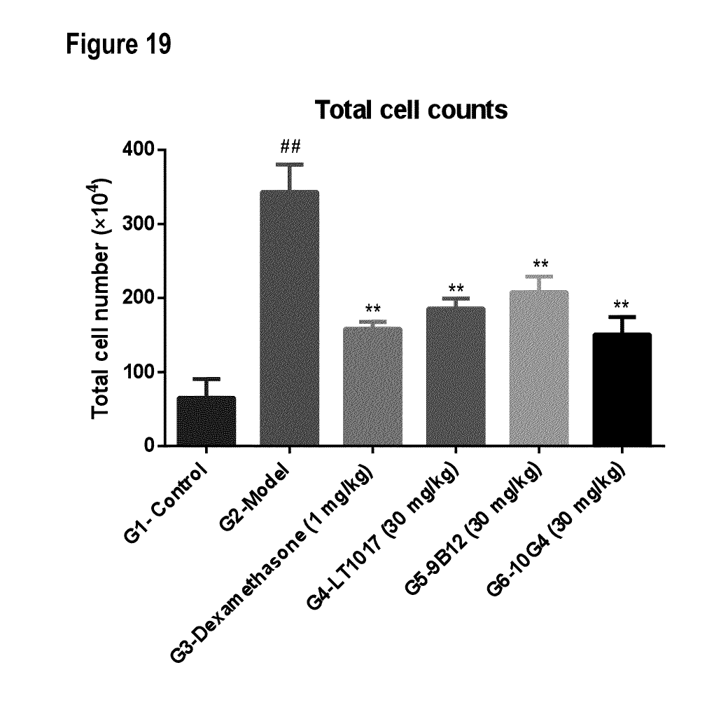

BAL fluid was centrifuged at 4° C. with 300 g×5 min and cells were suspended by 0.3 mL PBS (PH=7.2) (containing 1% FBS). Differential cell counts (lymphocytes, eosinophils, macrophages and neutrophils) were made after staining with Wright-Giemsa. Total cell counts are shown in FIG. 19. Data are mean±SEM, ## p<0.01 compared vs Control (unpaired t-test), **p<0.01 compared vs Model group (One-way ANOVA/Dunnett's). Differential cell counts are shown in Table 21 below.

TABLE 21
BALF Cell Classification (*10⁴)

| | EOS | | Mac | | Neu | | Lym | |
|---|---|---|---|---|---|---|---|---|
| Group | Mean | SEM | Mean | SEM | Mean | SEM | Mean | SEM |
| Control | 0.55 | 0.27 | 63.95 | 24.61 | 0.36 | 0.12 | 0.54 | 0.16 |
| Model | 183.28## | 22.00 | 147.72# | 20.72 | 4.34## | 0.54 | 7.87## | 1.61 |
| Dexamethasone | 22.18** | 4.75 | 132.54 | 10.87 | 1.99** | 0.36 | 1.69** | 0.30 |
| (1 mg/kg) | | | | | | | | |
| LT1017 | 47.62** | 10.34 | 132.82 | 10.99 | 1.08** | 0.10 | 4.10 | 0.92 |
| (30 mg/kg) | | | | | | | | |
| 9B12 | 79.75** | 14.48 | 122.46 | 15.53 | 1.42** | 0.19 | 4.57 | 1.17 |
| (30 mg/kg) | | | | | | | | |
| 10G4 | 40.33** | 4.77 | 121.75 | 20.57 | 0.82** | 0.17 | 1.20** | 0.86 |
| (30 mg/kg) | | | | | | | | |

Data are mean ± SEM,
#p < 0.05,
##p < 0.01 compared vs Control Group (unpaired t-test),
**p < 0.01 compared vs Model group (One-way ANOVA/Dunnett's).

Mice in which asthma was induced (“model”) had the highest number of cells (of every cell type) in BAL fluid, as expected, and mice in which asthma was not induced (“Control”) had the lowest. Anti cysLT antibody 10G4 reduced cell counts comparably to the positive control Dexamethasone, while anti-cysLT antibody 9B12 had a lesser or comparable effect. Interestingly, the nonspecific antibody control LT1017 also significantly reduced cell number in BAL fluid.

### Example 17

### Crossreactivity of Anti-cysLT Monoclonal Antibodies

Murine monoclonal antibodies 2G9 and 10G4 and humanized monoclonal antibody LT5014 (humanized version of 10G4) were tested by competitive ELISA for their ability to bind the cysLTs LTC4, LTD4, LTE4, LTF4, LTB4, a series of modified leukotrienes, cysLT receptor antagonists, and additional compounds shown in Table 22.

Conjugates: 150 nanomoles each of LTE4 (Cayman #20410) and LTC4 (Cayman #20210) were dried down under argon. Each lipid was biotinylated at a ratio of 20:1 Biotin:lipid using Pierce EZ-link NHS-LC-LC-Biotin kit (Thermo #21343) according to manufacturer's instructions.

ELISA: 96-well ELISA plates (Greiner #655061) were coated with 100 μL/well of 0.5 μg/mL capture antibody (5014: Goat anti-Human IgG, Fcγ specific, Jackson ImmunoResearch (West Grove Pa.) #109-005-098; 10G4 and 2G9: Goat anti-Mouse IgG, Fcγ specific Jackson #115-005-071) in 0.1 M carbonate buffer pH 9.5. Plates were sealed with thermal adhesive and allowed to incubate at 4° C. overnight. Plates were washed three times with 1×PBS (OmniPur, Thomas Scientific, Swedesboro N.J., Cat #650) 4+0.05% Tween-20 (Sigma # P1379) and then blocked with 150 μL/well of 1% BSA (Calbiochem, San Diego Calif., #126575) in 1×PBS+0.1% Tween-20 for 1 hour at room temperature. The plates were washed and 100 μL/well of anti-cysLT antibody in 1×PBS (LT5014: 100 ng/mL, Lot LP121468; 10G4: 50 ng/mL, Lot 121429; 2G9: 50 ng/mL, Lot LP121453) was added to the plate and allowed to incubate for 1 hour at room temperature. The plates were then washed three times with 1×PBS+0.05% Tween-20. All reference and test competitors were purchased from Cayman Chemicals with the exception of L-cysteine (Pierce #44889), and L-cysteine-L-glycine (Sigma #C0166-25MG). A 12 point, three-fold dilution series of reference competitor starting at 1 uM LTC4 or 10 uM LTE4 was used for 10G4/LT5014 or 2G9, respectively. A 12 point, three-fold dilution series of test competitors starting at 30 uM was used to evaluate cross-reactivity for all three antibodies, 2G9, 10G4 and LT5014. All competition reaction mixtures contained 0.5 nM conjugate (LTC4-LC-LC-Biotin, 10G4 and LT5014; LTE4-LC-LC-Biotin, 2G9) in 0.5×PBS+1 mg/mL BSA. 100 μL/well of diluted competitor/conjugate solution was applied to the plate and allowed to incubate for 21 hours at room temperature. The plates were washed and 100 μL/well of 1:60K dilution of secondary antibody in blocking solution (Peroxidase-conjugated streptavidin Jackson #016-030-084) was allowed to incubate on the plates for 15 minutes. The plates were washed and developed by allowing 100 μL/well cold TMB (Invitrogen #T0440-1L) to incubate on the plates for approximately 5 minutes. Reaction was stopped by addition of 100 μL/well 1.0M H2SO4. Plates were read at 450 nm on Perkin Elmer (Akron Ohio) plate reader (#1420). Results are calculated as ratios of the IC50 of the test competitor to the IC50 of the reference competitor (LTC4, LTD4 or LTE4).

Table 22 summarizes the crossreactivities of anti-cysLT antibodies LT5014, 10G4 and 2G9, with a variety of leukotriene and other potential ligands, expressed as percent of binding to LTC4, LTC4 and LTE4, respectively.

TABLE 22
Crossreactivity of anti-cysLT antibodies 2G9,
10G4 and LT5014

| | % Cross reactivity | | |
|---|---|---|---|
| Target | LT5014 | 10G4 | 2G9 |
| leukotriene C₄ | 100.0 | 100.0 | 3.8 |
| leukotriene D₄ | 4.1 | 4.0 | 5.4 |
| leukotriene E₄ | 0.6 | 0.3 | 100.0 |
| leukotriene F₄ | 76.6 | 37.3 | 750.7 |
| N-acetyl leukotriene E₄ | 27.6 | 11.4 | 480.5 |
| 14,15-leukotriene C₄ | <0.1 | <0.1 | 0.2 |
| 14,15-leukotriene E₄ | <0.1 | <0.1 | 6.4 |
| 11-trans leukotriene C₄ | <0.1 | 0.9 | 6.0 |
| leukotriene E₄ methyl ester | <0.1 | <0.1 | 6.7 |
| leukotriene C₄ methyl ester | 1.7 | 1.5 | 0.2 |
| Montelukast | <0.1 | <0.1 | <0.1 |
| Pranlukast | <0.1 | <0.1 | <0.1 |
| BayCysLT₂ | <0.1 | <0.1 | <0.1 |
| HAMI3379 | <0.1 | <0.1 | <0.1 |
| BAY-u9773 | <0.1 | <0.1 | 0.4 |
| Zafirlukast | <0.1 | <0.1 | <0.1 |
| MK 571 | <0.1 | <0.1 | <0.1 |
| leukotriene B₄ | <0.1 | <0.1 | <0.1 |
| 5(S)-HETE | <0.1 | <0.1 | <0.1 |
| 12(S)-HETE | <0.1 | <0.1 | <0.1 |
| 20-HETE | <0.1 | <0.1 | <0.1 |
| Prostaglandin E₂ | <0.1 | <0.1 | <0.1 |
| L-cysteine-HCl-H₂O | <0.1 | <0.1 | <0.1 |
| L-cysteine-L-glycine | <0.1 | <0.1 | <0.1 |
| L-Glutathione, reduced | <0.1 | <0.1 | <0.1 |
| L-Glutathione, oxidized | <0.1 | <0.1 | <0.1 |

It can be seen that the humanized antibody LT5014 has a similar crossreactivity profile to its murine parent, 10G4. In contrast, murine antibody 2G9 has a distinct binding profile for LTC4, LTD4 and LTE4, showing strongly preferential binding for LTE4 over LTC4 or LTD4 as noted in previous examples. Surprisingly, 2G9 was found to bind N-acetyl leukotriene E4 and leukotriene F4 with higher affinity than it binds to LTE4. N-acetyl LTE4 is a metabolite of LTE4 found in bile and is believed to be less biologically active than LTC4. All three antibodies showed minimal to undetectable binding to cysLT receptor antagonists (Montelukast, Pranlukast, BayCysLT2, HAMI3379, Bay-u9773, Zafirlukast, MK571), to leukotriene B, HETEs, prostaglandin E, L-cysteines or L-glutathione.

Thus the monoclonal antibodies 10G4 and its humanized version LT5014 are cysLT inhibitors which bind to LTC4, LTD4 and LTE4, with preferential binding to LTE4 (relative affinity 100:4.1:0.6% for LT5014). Both also bind LTF4 to a significant extent, and also N-acetyl LTE4. In contrast, antibody 2G9 preferentially binds LTE4 among the cysLTs (LTC4:LTD4:LTE4=100:5.4:3.8) but was surprisingly found to bind LTF4 and N-acetyl LTE4 with even higher affinity.

All of the compositions and methods described and claimed herein can be made and executed without undue experimentation in light of the present disclosure. While the compositions and methods of this invention have been described in terms of preferred embodiments, it will be apparent to those of skill in the art that variations may be applied to the compositions and methods. All such similar substitutes and modifications apparent to those skilled in the art are deemed to be within the spirit and scope of the invention as defined by the appended claims.

All patents, patent applications, and publications mentioned in the specification are indicative of the levels of those of ordinary skill in the art to which the invention pertains. All patents, patent applications, and publications, including those to which priority or another benefit is claimed, are herein incorporated by reference to the same extent as if each individual publication was specifically and individually indicated to be incorporated by reference.

The invention illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

What is claimed is:

1. A method of treating a disease or condition associated with aberrant levels of one or more cysteinyl leukotriene (cysLT) species, comprising administering to a subject having a disease or condition associated with aberrant levels of one or more cysLT species an effective amount of an antibody or fragment thereof that binds one or more cysLT species, whereby said disease or condition is treated.

2. The method of claim 1 wherein the disease or condition is selected from the group consisting of an inflammatory disease or condition, an allergy, a cardiovascular disease or condition, a respiratory disease or condition, a central nervous system disease or condition, cancer, a skin condition, a gastrointestinal condition, and rheumatoid arthritis.

3. The method of claim 2 wherein the skin condition is urticaria or atopic dermatitis, wherein the gastrointestinal condition is colitis, wherein the respiratory disease or condition is asthma, aspirin-exacerbated respiratory disease (AERD), airway hyperresponsiveness, or allergic rhinitis, wherein the cardiovascular disease is aberrant vascular permeability, or wherein the central nervous system disease or disorder is stroke or traumatic brain injury.

4. The method of claim 1 wherein the antibody or fragment thereof is selected from the group consisting of a polyclonal antibody, a monoclonal antibody, and a cysLT-binding fragment of one of the foregoing.

5. The method of claim 1 wherein the antibody or fragment thereof binds one or more of LTC4, LTD4, or LTE4.

6. The method of claim 5 wherein the antibody or fragment thereof detectably binds LTC4, LTD4, and LTE4.

7. The method of claim 5 wherein the antibody or fragment thereof preferentially binds LTC4 or LTE4.

8. A method of decreasing inflammation in a subject, comprising administering to the subject an effective amount of an antibody or fragment thereof that binds one or more cysLT species, whereby said inflammation is decreased.

9. The method of claim 8 wherein said inflammation affects the airway, skin, gastrointestinal tract, nervous system, joints, or blood vessels of the subject.

10. An isolated antibody, or antigen-binding fragment thereof, that binds one or more cysteinyl leukotriene (cysLT) species under physiological conditions and comprises at least one immunoglobulin heavy chain variable domain and at least one immunoglobulin light chain variable domain, wherein:
each immunoglobulin heavy chain variable domain comprises first, second, and third heavy chain complementarity determining regions (CDRs), wherein the first heavy chain CDR comprises an amino acid sequence selected from the group consisting of SEQ ID NO: 8, 14, 15, 16, 22, 23, 24, 31, 74, and an amino acid sequence having at least about 76% identity to SEQ ID NO: 24 or 31; the second heavy chain CDR comprises an amino acid sequence selected from the group consisting of SEQ ID NO: 9, 17, 25, 32, and an amino acid sequence having at least about 76% identity to SEQ ID NO: 25 or 32; and the third heavy chain CDR comprises an amino acid sequence selected from the group consisting of SEQ ID NO: 10, 18, 26, 33, and an amino acid sequence having at least about 76% identity to SEQ ID NO: 26 or 33; and
each immunoglobulin light chain variable domain comprises first, second, and third light chain CDRs, wherein the first light chain CDR comprises an amino acid sequence selected from the group consisting of SEQ ID NO: 11, 19, 27, 30, 34, 75, and an amino acid sequence having at least about 76% identity to SEQ ID NO: 27, 30, or 34; the second light chain CDR comprises an amino acid sequence selected from the group consisting of SEQ ID NO: 12, 20, 28, 35, and an amino acid sequence having at least about 76% identity to SEQ ID NO: 28 or 35; and the third light chain CDR comprises an amino acid sequence selected from the group consisting of SEQ ID NO: 13, 21, 29, 36, 76, and an amino acid sequence having at least about 76% identity to SEQ ID NO: 29 or 36.

11. An antibody or antigen-binding antibody fragment of claim 10 wherein the variable domain of each immunoglobulin heavy chain comprises an amino acid sequence selected from the group consisting of SEQ ID NO: 43, 45, 47, 50, 54, 58, 59, 61, 62, 63, 64, and 65, and the variable domain of each immunoglobulin light chain comprises an amino acid sequence selected from the group consisting of SEQ ID NO: 44, 46, 48, 49, 51, 66, 68, 69, 70, 71, 72, and 73.

12. The antibody or antigen-binding fragment of claim 10 that is a monoclonal antibody, a humanized monoclonal antibody, or an antigen-binding fragment of one of the foregoing.

13. The antibody or antigen-binding fragment of claim 10 in a pharmaceutically acceptable carrier.

14. An ELISA kit comprising an anti-cysLT antibody, or fragment thereof, according to claim 10.

15. A composition comprising leukotriene E4 (LTE4) covalently bound to blue carrier protein.

16. An antibody raised by immunizing an immune-competent mammal with an immunogen comprising the composition of claim 15.

17. An isolated nucleic acid that encodes an immunoglobulin heavy chain variable domain comprising first, second, and third heavy chain complementarity determining regions (CDRs), wherein the first heavy chain CDR comprises an amino acid sequence selected from the group consisting of SEQ ID NO: 8, 14, 15, 16, 22, 23, 24, 31, 74, and an amino acid sequence having at least about 76% identity to SEQ ID NO: 24 or 31; the second heavy chain CDR comprises an amino acid sequence selected from the group consisting of SEQ ID NO: 9, 17, 25, 32, and an amino acid sequence having at least about 76% identity to SEQ ID NO: 25 or 32; and the third heavy chain CDR comprises an amino acid sequence selected from the group consisting of SEQ ID NO: 10, 18, 26, 33, and an amino acid sequence having at least about 76% identity to SEQ ID NO: 26 or 33.

18. An isolated nucleic acid that encodes an immunoglobulin light chain variable domain comprising first, second, and third light chain CDRs, wherein the first light chain CDR comprises an amino acid sequence selected from the group consisting of SEQ ID NO: 11, 19, 27, 30, 34, 75, and an amino acid sequence having at least about 76% identity to SEQ ID NO:27, 30 or 34; the second light chain CDR comprises an amino acid sequence selected from the group consisting of SEQ ID NO: 12, 20, 28, 35, and an amino acid sequence having at least about 76% identity to SEQ ID NO: 28 or 35; and the third light chain CDR comprises an amino acid sequence selected from the group consisting of SEQ ID NO: 13, 21, 29, 36, 76, and an amino acid sequence having at least about 76% identity to SEQ ID NO: 29 or 36.

19. A vector or host cell that comprises an isolated nucleic acid according to one or more of claims 17 and 18.

20. A method for making an antibody, or antigen-binding fragment thereof, comprising cultivating a host cell according to claim 19 under conditions that allow synthesis of the antibody or antigen-binding fragment, thereby making antibody or antigen-binding fragment, wherein the method optionally further comprises isolating the antibody, or antigen-binding fragment thereof, so made.
